(19) United States
(12) Patent Application Publication
(10) Pub. No.: US 2007/0212330 A1
(43) Pub. Date: Sep. 13, 2007

(54) Nucleic Acid Molecules As Herpanase Potent Inhibitors, Compositions And Methods Of Use Thereof

(76) Inventors: Israel Vlodavsky, Mevasseret Zion (IL); Evgeny Edovitsky, Jerusalem (IL); Michael Elkin, Jerusalem (IL); Eyal Zcharia, Jerusalem (IL); Tamar Peretz, Jerusalem (IL)

Correspondence Address: FLEIT KAIN GIBBONS GUTMAN BONGINI & BIANCO, 21355 EAST DIXIE HIGHWAY, SUITE 115, MIAMI, FL, 33180, US

(21) Appl. No.: 11/569,794

(22) PCT Filed: Jun. 1, 2005
(86) PCT No.: PCT/IL05/00570

§ 371 Date: Jan. 4, 2007

(30) Foreign Application Priority Data
Jun. 1, 2004 (IL) 162276

(51) Int. Cl. C12N 15/11

(52) U.S. Cl. 424/93.1; 435/320.1; 435/325; 435/375; 514/44; 536/23.2

(57) ABSTRACT

Disclosed herein are ribonucleic acid molecules, specifically ribozymes and siRNAs, whose sequence is at least partially complementary to heparanase mRNA. Said molecules may thus be used for the specific inhibition of heparanase, and as therapeutics for pathologic conditions associated with heparanase expression, like for example tumor formation, progression and metastasis, tumor-associated angiogenesis, inflammatory disorders, kidney disorders and autoimmune disorders. Vectors, cells and compositions comprising said ribonucleic acid molecules are also disclosed herein.

### FIELD OF THE INVENTION

The present invention relates to inhibitors of heparanase expression. More particularly, the invention relates to ribozymes and siRNA molecules specific for heparanase, which are capable of inhibiting the expression of heparanase and thereby prevent heparanase related disorders.

### BACKGROUND OF THE INVENTION

The glycosaminoglycan heparan sulfate (HS) is the principal polysaccharide component of the basement membrane (BM). BM is a specialized type of the extracellular matrix (ECM), underlying endothelial and epithelial cell layers in all tissues and organs. In the blood vessel wall, BM functions as a scaffold for cellular architecture and integrity of the endothelium. Enzymatic remodeling of the BM barrier is a prerequisite for extravasation of leucocytes during inflammation, as well as of plasma macromolecules [Black, C. A. (1999) Dermatol Online 5:7]. HS is composed of repeating disaccharide units, which form linear chains covalently bound to a core protein [Timpl, R. (1996) Curr. Opin. Cell Biol. 8:618-24]. HS chains interact through specific attachment sites with the main protein components of BM, such as collagen IV, laminin and fibronectin. In addition, HS moieties in the ECM are responsible for specific binding of members of the heparin-binding family of growth factors (i.e., bFGF, VEGF, KGF, HGF) and serve as their extracellular reservoir [Timpl (1996) id ibid.; Vlodavsky, I. et al. (1991) Trends in Biochein. Sci. 16:268-71]. Thus, ECM-resident HS-bound growth and angiogenic factors are protected, stabilized and sequestered from their site of action, and can be readily mobilized to induce growth factor dependent processes (like for example neovascularization and tumor growth).

HS molecules are also associated with the cell surface via their core protein, and are important mediators of cell adhesion [Bernfield, M. et al. (1999) Annu. Rev. Biochem. 68:729-777] Cleavage of HS is, therefore, believed to result in disassembly of extracellular barriers, promoting both cell invasion and release of HS-bound bioactive molecules (i.e. angiogenic and growth promoting factors), hence playing a decisive role in tumor invasiveness, metastatic spread and angiogenesis [Vlodavsky, I. (1991) id ibid.; Ishai-Michaeli, R. et al. (1990) Cell Regul. 1:833-42].

The mammalian endoglycosidase heparanase is the predominant enzyme degrading HS [Vlodavsky, I. et al. (1999a) Nat. Med. 5:793-802; Hulett, M. D. et al. (1999) Nat. Med. 5:803-9; Kussie, P. H. et al. (1999) Biochem. Biophys. Res. Commun. 261:183-7; Toyoshima, M. and Nakajima, M. J. (1999) Biol. Chem. 274:24153-60]. Heparanase activity is likely to be involved in fundamental biological processes associated with ECM disintegration and cell migration, ranging from pregnancy, morphogenesis and normal development, to inflammation, angiogenesis, and cancer metastasis [Vlodavsky (1999a) id ibid.; Vlodavsky, I. et al. (1994) Invasion Metastasis 14:290-302 (1994); Dempsey, L. A. et al. (2000) Glycobiology 10:467-75; Nakajima, M. et al. (1988) J. Cell. Biochem. 36:157-67; Parish, C. R. et al. (2001) Biochim. Biophys. Acta 1471:M99-M108].

Heparanase mRNA and protein are preferentially expressed in metastatic cell lines and human tumor tissues [Vlodavsky (1999a) id ibid.; Hulett (1999) id ibid.; Kussie (1999) id ibid.; Nakajima (1988) id ibid.; Parish, (2001) id ibid.; Friedmann, Y. et al. (2000) Am. J. Pathol. 157:1167-75]. Moreover, enhanced heparanase mRNA expression correlates with reduced postoperative survival of cancer patients [Gohji, K. et al. (2001) J. Urol. 166:1286-90; Koliopanos, A. et al. (2001) Cancer Res. 61:4655-9]. Overexpression of the heparanase cDNA in low metastatic tumor cells confers a high metastatic potential in experimental animals [Vlodavsky (1999a) id ibid.]. The heparanase enzyme has also been shown to elicit an angiogenic response by means of releasing ECM-resident HS-bound angiogenic factors [Elkin, M. et al. (2001) Faseb. J. 15:1661-3]. A pronounced correlation between heparanase expression and tumor microvessel density has been reported [Gohji (2001) id ibid.; Watanabe, M. et al. (2003) Gynecol. Obstet. Invest. 56:77-82; Kelly, T. et al. (2003) Cancer Res. 63:8749-56 (2003)]. Heparin, other polysaccharides and heparin-mimicking molecules which inhibit heparanase enzymatic activity also reduce the incidence of metastasis in experimental animals [Hulett (1999) id ibid.; Vlodavsky (1994) id ibid.; Nakajima (1988) id ibid.; Miao, H. Q. et al. (1999) Int. J. Cancer 83:424-31; Parish, C. R. et al. (1999) Cancer Res. 59:3433-41]. However, the use of these pluripotent compounds remains questionable due to the lack of specificity [Borsig, L. et al. (2001) Proc. Natl. Acad. Sci. USA. 98:3352-7; Koenig, A. et al. (1998) J. Clin. Invest. 101:877-89].

Possible involvement of heparanase in inflammation has also been addressed, emphasizing the contribution of heparanase residing in activated cells of the immune system [Vaday G. G. and O. Lider. (2000) J Leukoc Biol 67:149-159; Vlodavsky (1992) id ibid.; Lider, O. et al. (1990) Eur. J. Immunol. 20:493-499; Lider, O. et al. (1989) J. Clin. Invest. 83:752-756; Matzner, Y. et al. (1985) J. Clin. Invest. 76:1306-1313; Fridman, R. et al. (1987) J. Cell. Physiol. 130:85-92]. The exact role of heparanase in the inflammatory process remains unclear. Prior to the cloning of the heparanase gene, it was shown that inhibition of T lymphocyte-derived heparanase by species of heparin inhibits T cell migration and T cell-mediated immunity [Lider (1990) id ibid.; Lider (1989) id ibid.; Sy, M. S. et al. (1983) Cell Immunol. 82:23-32]. The causative involvement of heparanase in this system was, however, questionable because of the multiple biological activities of heparin [Koenig (1998) id ibid.; Borsig (2001) id ibid.]. At the same time it was reported that degradation products reportedly released by heparanase from the ECM, inhibit delayed-type hypersensitivity (DTH) reactivity in mice [Lider, O. et al. (1995) Proc. Natl. Acad. Sci. USA 92:5037-5041].

DTH is an important in vivo manifestation of cell-mediated immune responses. The development of DTH involves recruitment and activation of antigen-specific T cells, synthesis of a cascade of chemotactic and activating cytokines, recruitment of antigen-nonspecific effector cells, fibrin deposition, and increased vascular permeability. This is followed, similar to other types of inflammatory responses, by translocation of leukocytes, including monocytes, neutrophils and T lymphocytes, from the vascular system, through extracellular tissue barriers, into the site of inflammation Sub-endothelial BM represents the major physical obstacle for leukocyte extravasation and entry into inflammatory sites.

The present invention discloses alternative strategies for heparanase inhibition, applying gene-silencing technologies which specifically suppress heparanase expression in vitro and in vivo, and thereby demonstrate its causal involvement in cancer invasion, metastasis and angiogenesis, as well as in inflammation.

The inventors first utilized a ribozyme approach, well known to be highly effective in gene silencing [Sigurdsson, S. T. et al. (1995) Trends in Biotechnol. 13:286-9].

The inventors further applied the RNA interfering (RNAi) technology, recognized as a highly effective approach for gene silencing and characterized by increased stability, specificity and potential therapeutic application [Sharp, P. A. (2001) Genes Dev. 15:485-90; Elbashir, S. M. et al. (2001a) Nature 411:494-8].

The present invention clearly demonstrates inhibition of tumor angiogenesis and metastasis by two different heparanase silencing approaches and establishes the decisive role of heparanase in tumor progression and inflammation. Moreover, the results of the present invention provide novel molecular tools to better elucidate the involvement of heparanase in normal and pathological processes, and for potential therapeutic intervention in these processes.

Therefore, it is an object of the invention to provide nucleic acid molecules having catalytic activity specific for heparanase (targeted against the heparanase molecule). More specifically, the invention provides specific ribozyme molecules and siRNA targeted against mouse and human heparanase. In yet another object, the invention provides compositions and methods for the inhibition of heparanase gene expression and thereby provides pharmaceutical compositions and methods of treatment of heparanase associated disorders.

These and other objects of the invention will become apparent as the description proceeds.

### SUMMARY OF THE INVENTION

Thus, in a first aspect, the invention relates to a nucleic acid molecule comprising at least one target specific sequence, which is complementary to a target ribonucleotide sequence comprised within heparanase mRNA. The nucleic acid molecule of the invention is capable of inhibiting the expression of heparanase.

According to one preferred embodiment, the nucleic acid molecule of the invention may be a ribonucleic acid molecule selected from the group consisting of a ribonucleic acid molecule having endonuclease activity and a small interfering RNA (siRNA).

According to a specific embodiment, where the nucleic acid molecule of the invention is a ribonucleic acid molecule having endonuclease activity, such molecule may preferably be a ribozyme, more preferably a hammerhead ribozyme, which specifically cleaves heparanase RNA and thereby inhibits the expression of heparanase.

More particularly, the ribozyme of the invention may comprise three contiguous regions, a first region, a second region and a third region, where at least a portion of the first and the third regions are complementary to target RNA sequences within heparanase, and at least a portion of the second region is a ribozyme catalytic domain.

Specifically, the ribozyme of the invention comprises a ribonucleic acid sequence selected from the group consisting of SEQ ID NO: 19, 20, 21, 22, 23, 24 and any derivatives or functional fragments thereof.

A particular ribozyme of the invention comprises the ribonucleic acid sequence denoted by SEQ ID NO: 19 or any analog, variant, derivative and fragment thereof. Preferably, the ribozyme of the invention has the ribonucleic acid sequence as denoted by SEQ ID NO: 19 and is designated HpaRz2.

According to an alternative embodiment, the ribonucleic acid molecule of the invention is a siRNA comprising a double strand ribonucleic acid (dsRNA) sequence, wherein at least a portion of one strand of said dsRNA comprises a sequence complementary to a target sequence within the heparanase mRNA sequence.

Accordingly, the siRNA molecule of the invention leads to specific cleavage of heparanase RNA, thereby inhibiting heparanase expression.

In one specific embodiment, the siRNA of the invention comprises sequences complementary to target sequences derived from the mouse heparanase. These siRNA molecules therefore comprise a dsRNA sequence selected from the group consisting of a dsRNA composed of one strand comprising the sequence as denoted by SEQ ID NO: 26 and a second complementary strand comprising the sequence as denoted by SEQ ID NO: 27, and a dsRNA composed of one strand comprising the sequence as denoted by SEQ ID NO: 28 and a second complementary strand comprising the sequence as denoted by SEQ ID NO: 29.

More specifically, according to this embodiment the siRNA is designated si1 and composed of one strand having the sequence as denoted by SEQ ID NO: 26, or any functional derivatives or fragments thereof, and a second complementary strand having the sequence as denoted by SEQ ID NO: 27, or any functional derivatives or fragments thereof. Alternatively, the siRNA is designated si2 and composed of one strand having the sequence as denoted by SEQ ID NO: 28, or any functional derivatives or fragments thereof, and a second complementary strand having the sequence as denoted by SEQ ID NO: 29, or any functional derivatives or fragments thereof.

In another specific embodiment, the siRNA of the invention comprises sequences complementary to target sequences derived from the human heparanase. These siRNA molecules therefore comprise a dsRNA sequence selected from the group consisting of a dsRNA composed of one strand comprising the sequence as denoted by SEQ ID NO: 30 and a second complementary strand comprising the sequence as denoted by SEQ ID NO: 31 and a dsRNA composed of one strand comprising the sequence as denoted by SEQ ID NO: 32 and a second complementary strand comprising the sequence as denoted by SEQ ID NO: 33.

More specifically, according to this embodiment the siRNA is designated siRNA-H1 and is composed of one strand having the sequence as denoted by SEQ ID NO: 30, or any functional derivatives or fragments thereof, and a second complementary strand having the sequence as denoted by SEQ ID NO: 31, or any functional derivatives or fragments thereof. Alternatively, the siRNA is designated siRNA-H2 and composed of one strand having the sequence as denoted by SEQ ID NO: 32 and a second complementary strand having the sequence as denoted by SEQ ID NO: 33.

According to a second aspect, the invention relates to an expression vector comprising a polynucleotide sequence encoding a nucleic acid molecule which comprises at least one target specific sequence complementary to a target ribonucleotide sequence comprised within heparanase mRNA. The vector of the invention allows or promotes the expression of said nucleic acid molecule in a manner which is capable of inhibiting the expression of heparanase. The vector of the invention may optionally further comprise at least one of an operably linked promoter, a transcription start region, a transcription termination region and further regulatory elements.

According to a specifically preferred embodiment, the expression vector of the invention may comprise a polynucleotide sequence encoding any of the nucleic acid molecules defined by the invention. More particularly, the invention provides for an expression vector encoding any of the ribozymes or the siRNAs of the invention

Still further, the invention provides a host cell transformed or transfected with an expression vector of the invention.

In a third aspect, the invention relates to a composition for the inhibition of heparanase expression, comprising as an active ingredient at least one isolated and purified nucleic acid molecule comprising at least one target specific sequence, which sequence is complementary to a target ribonucleotide sequence comprised within heparanase mRNA. The composition of the invention optionally further comprises a pharmaceutically acceptable carrier, diluent, excipient and/or additive.

According to a specifically preferred embodiment, the composition of the invention comprises as active ingredient any one of the isolated and purified nucleic acid molecules of the invention, the expression vectors encoding such nucleic acid molecules or any host cell transformed or transfected with such vectors, and thereby expressing any of the nucleic acid molecules of the invention.

According to one specific embodiment, a preferred composition of the invention comprises as active ingredient at least one ribozyme, which ribozyme has the ribonucleic acid sequence as denoted by SEQ ID NO: 19 and is designated HpaRz2.

Another specifically preferred composition of the invention comprises as active ingredient at least one siRNA molecule composed of one strand having the sequence as denoted by SEQ ID NO: 26 and a second complementary strand having the sequence as denoted by SEQ ID NO: 27, designated si1. Alternatively, the composition of the invention comprises as active ingredient at least one siRNA molecule composed of one strand having the sequence as denoted by SEQ ID NO: 28 and a second complementary strand having the sequence as denoted by SEQ ID NO: 29, designated si2.

According to another embodiment, the composition of the invention comprises as active ingredient at least one siRNA molecule composed of one strand having the sequence as denoted by SEQ ID NO: 30 and a second complementary strand having the sequence as denoted by SEQ ID NO: 31, designated siRNA-H1. An alternative composition of the invention comprises as active ingredient at least one siRNA molecule composed of one strand having the sequence as denoted by SEQ ID NO: 32 and a second complementary strand having the sequence as denoted by SEQ ID NO: 33, designated siRNA-H2.

The compositions of the invention may be for medical use.

Thus, the invention further provides a pharmaceutical composition for the treatment or the inhibition of a process or a pathologic disorder associated with heparanase expression or overexpression, comprising as active ingredient at least one nucleic acid molecule as defined above, i.e. a nucleic acid molecule comprising at least one target specific sequence, which sequence is complementary to a target ribonucleotide sequence comprised within heparanase mRNA. The pharmaceutical composition of the invention optionally further comprises a pharmaceutically acceptable carrier, diluent, excipient and/or additive.

According to a specifically preferred embodiment, the pharmaceutical composition of the invention comprises as active ingredient at least one of a nucleic acid molecule of the invention, an expression vector encoding such nucleic acid molecule and a host cell transformed or transfected with such vectors and thereby expressing any of the nucleic acid molecules of the invention.

According to one specific embodiment, a preferred pharmaceutical composition of the invention comprises as active ingredient at least one ribozyme, which ribozyme is designated HpaRz2 and has the ribonucleic acid sequence as denoted by SEQ ID NO: 19.

Another specifically preferred pharmaceutical composition of the invention comprises as active ingredient at least one siRNA molecule composed of one strand having the sequence as denoted by SEQ ID NO: 26 and a second complementary strand having the sequence as denoted by SEQ ID NO: 27, designated si1. An alternative pharmaceutical composition of the invention comprises as active ingredient at least one siRNA molecule composed of one strand having the sequence as denoted by SEQ ID NO: 28 and a second complementary strand having the sequence as denoted by SEQ ID NO: 29, designated si2.

According to another embodiment, the pharmaceutical composition of the invention comprises as active ingredient at least one siRNA molecule composed of one strand having the sequence as denoted by SEQ ID NO: 30 and a second complementary strand having the sequence as denoted by SEQ ID NO: 31, designated siRNA-H1. In yet another pharmaceutical composition of the invention comprises as active ingredient at least one siRNA molecule composed of one strand having the sequence as denoted by SEQ ID NO: 32 and a second complementary strand having the sequence as denoted by SEQ ID NO: 33, designated siRNA-H2.

According to one preferred embodiment, the pharmaceutical composition of the invention is intended for the treatment and inhibition of a process associated with heparanase expression, wherein said process may be for example any one of angiogenesis, tumor formation, tumor progression and tumor metastasis.

In yet another embodiment, the pharmaceutical composition of the invention may be particularly useful for the treatment and/or the inhibition of a pathologic disorder associated with heparanase expression, such as a malignant proliferative disorder. More specifically, such malignant proliferative disorder may be any one of solid and non-solid tumor selected from the group consisting of carcinoma, sarcoma, melanoma, leukemia and lymphoma.

Alternatively, the pharmaceutical composition of the invention may be used for the treatment of pathologic disorders such as inflammatory disorder, kidney disorder and autoimmune disorder.

The present invention further provides the use of any one of the isolated and purified nucleic acid molecules of the invention, the expression vectors encoding such nucleic acid molecules or any of the host cells of the invention as an agent for the inhibition of heparanase expression.

Furthermore, the invention provides for the use of any one of the isolated and purified nucleic acid molecules of the invention, the expression vectors encoding such nucleic acid molecules or any of the host cells of the invention, in the preparation of a composition for the inhibition of heparanase expression.

Still further, the invention relates to the use of any one of the isolated and purified nucleic acid molecules of the invention, the expression vectors encoding such nucleic acid molecules or any of the host cells of the invention, in the preparation of a pharmaceutical composition for the treatment or the inhibition of a process or a pathologic disorder associated with heparanase expression. More specifically, a process associated with heparanase expression may be any one of angiogenesis, tumor formation, tumor progression and tumor metastasis. A pathologic disorder associated with heparanase expression may be a malignant proliferative disorder, for example, a solid and non-solid tumor selected from the group consisting of carcinoma, sarcoma, melanoma, leukemia and lymphoma, or alternatively, any one of inflammatory disorder, kidney disorder and autoimmune disorder.

In a further aspect, the invention relates to a method for the inhibition of heparanase expression comprising the step of in viuo or in vitro contacting heparanase RNA molecules, under suitable conditions, with an inhibitory effective amount of a nucleic acid molecule of the invention, an expression vector as defined by the invention, a host cell transformed or transfected with such expression vector or with a composition comprising the same.

The invention further provides a method for the inhibition of heparanase expression in a subject in need thereof, wherein said method comprises the step of administering to said subject an inhibitory effective amount of a nucleic acid molecule as defined by the invention, an expression vector comprising said nucleic acid sequence, a host cell transformed or transfected with said expression vector or a composition comprising the same.

Still further, the invention provides a method for the inhibition or the treatment of a process or a pathologic disorder associated with heparanase expression, wherein said method comprises the step of administering to a subject in need thereof a therapeutically effective amount of a nucleic acid molecule as defined by the invention, an expression vector comprising said nucleic acid sequence, a host cell transformed or transfected with said expression vector or a composition comprising the same.

According to one preferred embodiment, the method of the invention is intended for the treatment of a process associated with heparanase expression, such as for example angiogenesis, tumor formation, tumor progression and tumor metastasis. The method of the invention is specifically suitable for the treatment of a pathologic disorder associated with heparanase expression, for example a malignant proliferative disorder, such as solid and non-solid tumor selected from the group consisting of carcinoma, sarcoma, melanoma, leukemia and lymphoma, an inflammatory disorder, an autoimmune disorder or kidney disorders. In particular, the method of the invention is specifically suitable for the treatment of DTH.

### BRIEF DESCRIPTION OF THE FIGURES

FIG. 1A-1C: Structure and in vitro activity of anti-heparanase hammerhead ribozyme

FIG. 1A: DNA template (sense strand; SEQ ID NO:55) of anti-heparanase ribozyme (HpaRz2), containing a T7 promoter, two complementary substrate-specific sequences, and an invariable catalytic consensus domain.

FIG. 1B: Schematic representation of HpaRz2 (SEQ ID NO:19). After in vitro transcription, the catalytic core forms the typical hammerhead structure due to base pair formation between complementary nucleotides. The flanking, substrate-specific sequences bind the hpa RNA substrate (SEQ ID NO:56). Arrow: predicted cleavage site.

FIG. 1C: In vitro cleavage assay using HpaRz2. The radioactively labeled hpa RNA substrate was mixed with the HpaRz2 at a molecular ratio of 1:50. The RNA substrate was also incubated without ribozyme (−Rz), as a negative control. The mixtures were incubated for 15 or 60 minutes at 45° C. The full-length substrate (1477 nt) and cleavage products were separated by electrophoresis in polyacrylamide-gel and visualized by autoradiography.

Abbreviations: prom. (promoter), Sub. Spec. seq. (substrate specific sequence), cat. Dom. (catalytic domain), min. (minute).

FIG. 2A-2C: Effect of HpaRz2 on endogenous heparanase mRNA, enzymatic activity and invasiveness of MDA-435 cells

FIG. 2A: Hpa mRNA levels in MDA-435 cells stably transfected with HpaRz2 or ContRz, assessed by semi-quantative RT-PCR with primers specific for human heparanase. RT-PCR products obtained with GAPDH specific primers were used as a control for equal RNA loading (Inset). Heparanase activity: MDA-435 cells stably transfected with pHpaRz2 (∘) or pContRz (♦) were incubated with ³⁵S-labeled ECM for 5 h at 37° C. (pH 6.2). ³⁵S-labeled degradation fragments released into the incubation medium were analyzed by gel filtration on Sepharose 6B, as described in “Experimental procedures”.

FIG. 2B: Immunofluorescent staining of MDA-435 cells transfected with pHpaRz2 (Right) or pContRz (Left), with rabbit anti-heparanase polyclonal antibody # 733.

FIG. 2C: Invasion through Matrigel. MDA-435 stable transfected with pHpaRz2 or with pContRz were incubated (3×10⁵ cells/ml, 6 h, 37° C.) in DMEM containing 0.1% BSA on top of Matrigel-coated filters. The number of cells/field in the lower surface of the filter was determined, as described in “Experimental procedures”. Error bars show 95% confidence intervals (P value<0.0001).

Abbreviations: lab. Mat. (labeled material), Frac. (fraction), ce. Inva. (cell invasion), fie. (field).

FIG. 3A-3C: Effect of HpaRz2 on heparanase activity and lymphoma cell invasion and adhesion

FIG. 3A: Heparanase activity. cHpaEb lymphoma cells transfected with pHpaRz2 (∘) or pContRz (♦) were incubated with ³⁵S-labeled ECM for 5 h at 37° C. (pH 6.2). ³⁵S-labeled degradation fragments released into the incubation medium were analyzed by gel filtration on Sepharose 6B, as described in “Experimental procedures”.

FIG. 3B: Invasion through Matrigel. ³H-thymidine-labeled cHpaEb cells transfected with pHpaRz2 or pContRz were incubated (1×10⁶ cells/ml, 6 h, 37° C.) in RPMI medium containing 0.1% BSA on top of Matrigel-coated filters. After incubation, the upper surface of the filter was wiped free of cells and the extent of cell invasion was measured by counting in a β-scintillation counter, as described in “Experimental procedures”. Data are the means. Error bars show 95% confidence intervals of triplicate filters (P value<0.0001).

FIG. 3C: Cell adhesion. CHpaEb cells expressing active (HpaRz2) or control (ContRz) ribozymes were prelabeled with ³H-thymidine, suspended in RPMI medium, seeded on ECM and allowed to attach for 15 min at 37° C. The extent of cell adhesion was measured, as described in “Experimental procedures”. Data are the means. Experiments were performed at least 3 times and error bars show 95% confidence intervals (P value<0.0061).

Abbreviations: lab. Mat., labeled material; ce. Inva., cell invasion; ce. adh., cell adhesion.

FIG. 4A-4C: Effect of HpaRz2 on mortality, liver metastasis and tumor angiogenesis in cHpaEb mouse lymphoma model

FIG. 4A: Survival rate. CD1 nude mice were inoculated s.c. with 1×10⁶ cHpaEb lymphoma cells transfected with pHpaRz2 (▪) or pContRz (∘). Mice were monitored daily for survival time.

FIG. 4B: Infiltration of the liver tissue by lymphoma cells. On day 11 of the experiment, five mice of each group were sacrificed and their livers dissected and weighed. Top: Gross appearance. Middle: Mean weight (error bars show 95% confidence intervals) of livers derived from mice injected with pHpaRz2− (Right) vs. pContRz− (Left) transfected lymphoma cells (P value<0.0001). Bottom: Histological analysis of H&E-stained sections of liver tissue derived from mice injected with pHpaRz2− (Right) and pContRz− (left) transfected lymphoma cells, ×200. Arrows mark liver colonization by ContRz-expressing cHpaEb cells.

FIG. 4C: Primary tumor vascularization. Primary tumors produced by HpaRz2− or ContRz− transfected cHpaEb cells were excised on day 11, photographed and processed for histology. Top: gross appearance. Tumors produced by cHpaEb cells transfected with pContRz (left) appeared dark-reddish, as opposed to a pale appearance of tumors generated by pHpaRz2 transfected cells (right), reflecting a marked difference in vascularity, blood content, and hemorrhage. Middle: microvessel density in the primary tumor tissue. Five μm paraffin sections of tumor produced by cHpaEb cells transfected with pContRz (left) or pHpaRz2 (right) were stained with anti-Von Willebrand Factor antibody (reddish staining), ×200. Bottom: Vascular density (vessels per microscopic field) was determined, as described in “Experimental procedures”. Data are the means. Error bars show 95% confidence intervals, P value<0.0001 (Bottom).

Abbreviations: D. ce. inoc., days after cell inoculation; Liv. Wei., liver weight; Vess. nu./fie., vessel number/field.

FIG. 5A-5C: RNA interference inhibits B16-BL6 heparanase enzymatic activity, Matrigel invasion and lung colonization

FIG. 5A: Top: Hpa mRNA levels in B16-BL6 cells transfected with siRNA expression vectors pSi1, pSi2, or empty pSUPER vector (mock), assessed by semi-quantative RT-PCR with primers specific for mouse heparanase (upper panel). RT-PCR products obtained with L19 specific primers were used as a control for equal RNA loading (lower panel). Bottom: The intensity of each band was quantitated using the Scion Image program and the results are expressed as percent of band intensity relative to that of L19.

FIG. 5B: Heparanase activity. B16-BL6 cells transfected with siRNA pSi1 (Δ), pSi2 (∘), or empty (▪) vector, were incubated with ³⁵S-labeled ECM for 5 h at 37° C. (pH 6.2). The incubation medium was analyzed by gel filtration on Sepharose 6B, as described in “Experimental procedures”.

FIG. 5C: Invasion through Matrigel. B16-BL6 transfected with pSi1, pSi2, or with vector alone, were incubated (3×10⁵ cells/ml, 6 h, 37° C.) in DMEM containing 0.1% BSA on top of Matrigel-coated filters. The number of cells/field on the lower surface of the filter was determined, as described in “Experimental procedures”. Data are the means. Error bars show 95% confidence intervals (P value<0.0011).

FIG. 5D: Lung colonization. C57/BL6 mice were inoculated (i.v.) with B16-BL6 melanoma cells (3×10⁵ cells/mouse) transfected with either mock or pSi2 vectors. Fifteen days afterwards mice were sacrificed and their lungs were fixed in Bouin's solution and examined for the number of melanoma colonies on the lung surface. Data are the means. Error bars show 95% confidence intervals (P value<0.0001) (Top). Bottom: Gross appearance of lungs of mice inoculated with mock transfected (upper panel) vs. siRNA transfected (lower panel) B16-BL6 cells.

Abbreviations: lab. Mat., labeled material; ce. Inva., cell invasion; fie., field; mo., mock; colon., colonies; lu., lung; frac., fraction.

FIG. 6A-6B: Heparanase siRNA inhibits hair growth in vivo

FIG. 6A: siRNA-expression vector skin electroporation. Hair growth on the back of C57BL/6 mice was induced by depilation as described in Experimental procedures. Anti-heparanase pSi2 construct (Middle), empty vector (pSUPER) (Right) or pcDNA3-GFP plasmid (Left) were injected into skin and electroporated as described in Experimental procedures. Mice were examined for hair growth 96 h after electroporation.

FIG. 6B: siRNA-expressing lentivirus skin infection. Hair growth was induced by depilation and lentivirus containing anti-heparanase pSi2-Lenti (Right) or PBS (Left) was injected into skin. Mice were examined for hair growth a week after infection.

FIG. 7: Heparanase siRNA inhibits DTH reactivity in vivo

Female BALB/c mice were sensitized by application of oxazalone on the shaved abdominal skin as described in Experimental procedures. Five days later (day 0 of experiment) mice were challenged by oxazalone and electroporated with empty vector (pSUPER) or pSi2 as described in Experimental procedures. Thickness of a constant area of the ear was measured immediately before challenge, 24 hours after challenge and every other day for 5 days, as described in Experimental procedures.

Abbreviations: w/o, without; D., days.

FIG. 8: Increased DTH reactivity in heparanase overexpressing transgenic mice

DTH reactions were elicited in the left ear skin of hpa-tg mice and their wild-type counterparts using oxazolone. Right ears of the same animals were treated with vehicle alone. Swelling of the challenged ears is expressed in mm as the increase over the thickness measured in vehicle alone treated ears (which is considered as the baseline). Challenged ears in hpa-tg mice (Δ) showed a 3.5-fold increase in swelling over the baseline (▪), as compared to only 2-fold increase in wild-type mice (◯), 24 h after challenge with oxazolone. The differences between the two groups remained statistically significant for 3 days. The experiment was repeated twice, n=5 per experimental condition and time point. Data are expressed as mean±SD.

Abbreviations: D., days; E. th., ear thickness.

FIG. 9A-9B: Endogenous heparanase expression in vivo upon DTH induction.

Five days post sensitization, the left ear of 4 female BALB/c mice was treated with oxazolone and the right ear with vehicle alone. Ear tissues were harvested 24 h post challenge, and processed for immunohistochemical analysis of heparanase expression (reddish staining; sebaceous glands are positively stained in all samples, due to a non-specific absorption, as previously described [Philp, D. et al. (2004) Faseb J. 18:385-387]. Vascular structures were recognized as luminal or slit-like structures that occasionally contained blood cells and were delineated by flattened endothelial cells. Representative microphotographs are shown.

FIG. 9A: Non-challenged ear. Top: little or no heparanase-positive cells are detected in the dermis (magnification ×200). Bottom: capillary endothelial cells in the ear skin dermis are negative for heparanase staining (magnification ×1000).

FIG. 9B: Oxazolone challenged ear. Top: heparanase-expressing cellular structures are easily detected in the dermis (×200). Bottom: Higher magnification demonstrates expression of heparanase in capillary endothelial cells (×1000). Control sections stained using secondary antibody alone showed no staining.

FIG. 10A-10B: Effects of IFN-γ on heparanase expression in endothelial cells.

FIG. 10A: Semi-quantitative RT-PCR. EA.hy926 cells were incubated (16 h) in triplicates in the absence or presence of 80 mg/ml IFN-γ. RNA was then isolated from the cells and comparative semi-quantitative PCR was performed. Aliquots (10 82 l) of the PCR products were separated by 1.5% agarose gel electrophoresis and visualized (top). The intensity of each band was quantitated using Scion Image software and the results are expressed as band intensity relative to that of L19. The histogram bars represent the mean±SD (error bars) of three independent experiments (bottom).

FIG. 10B: Heparanase activity. EA.hy926 cells were incubated (16 h) in the absence (□), or presence (♦) of 80 mg/ml IFN-γ. Cell lysates were normalized for equal protein and incubated (4 h, pH 6.0, 37° C.) with sulfate labeled ECM. Labeled degradation fragments released into the incubation medium were analyzed by gel filtration on Sepharose 6B.

Abbreviations: cont., control; rat., ratio; lab. mat., labeled material; frac., fraction.

FIG. 11A-11B: Heparanase promoter is activated upon DTH elicitation.

The ears of oxazolone-sensitized Balb/c mice were electroporated with either Hpse-LUC or CMV-LUC reporter constructs. Left ears in both the experimental and control groups were challenged 24 h later, while right ears remained untreated. 48 h after challenge, when a pronounced DTH reaction was noted in the left, but not in the right ears of all mice (as judged by ear swelling and edema formation), the ears were dissected and lysed. Lysates were normalized for total protein content and luciferase activity was determined as described in “Experimental Procedures” section. Two independent experiments were performed, three mice per treatment. Graphs show LUC activity, represented by relative luciferase units (RUL).

FIG. 11A: Experimental group. Mice transfected with Hpse-LUC.

FIG. 11B: Control group. Mice transfected with CMV-LUC.

FIG. 12A-12C: Effect of anti-heparanase siRNA on DTH reactivity in vivo.

Ears of oxazolone-sensitized Balb/c mice were electroporated with anti-heparanase siRNA expression vector pSi2 (●); empty vector pSUPER (▴); or were not electroporated (♦), followed by challenge with the hapten 24 h later. Hapten was also applied on the ears of 5 additional mice, which were not previously sensitized or electroporated (▪). Three independent experiments were performed and 5 mice were used per treatment.

FIG. 12A: Mouse expressing CMV-LUC in the ear, demonstrating that the in vivo electroporation works.

FIG. 12B: Ear thickness was measured for 5 consecutive days post challenge. Arrows indicate time-point of application of siRNA electroporation (full arrow) or oxazalone (empty arrow).

FIG. 12C: The ears in which DTH was induced following electroporation with pSi2 (left) or pSUPER (right) vectors were harvested 24 h post challenge and processed for immunohistochemical analysis of heparanase expression (reddish staining; sebaceous glands are positively stained in all preparates, due to non-specific absorption as previously reported [Philp (2004) id ibid.]. Top: magnification ×200. Bottom: ×1000. Positively stained capillary endothelium is noted in the dermis of pSUPER, but not pSi2-electroporated ear skin.

FIG. 13A-B: Efffect of local heparanase silencing on vascular leakage and basement membrane integrity in the challenged ear skin.

Ears of five oxazolone-sensitized BALB/c mice were electroporated with anti-heparanase siRNA pSi2 (right), or empty pSUPER (left) vectors, 24h prior to induction of DTH reaction by application of oxazolone on the ears of both sides.

FIG. 13A: Evans blue dye was injected intravenously 16 h later. Unlike the massive Evans blue extravasation observed in pSUPER-electroporated ears, pSi2 electroporation almost halted vascular leakage, as visualized by the lack of extravasated dye.

FIG. 13B: Tissue sections taken from pSi2− (right) and pSUPER− (left) electroporated ears 24 h after challenge, were histologically processed and subjected to Masson-Trichrom staining. Excessive disruption (arrows) of the BM (blue) was seen in the capillary wall of pSUPER-electroporated ears (left), as compared to a continuous intact BM in the capillary wall of pSi2-electroporated ears (right). Magnification ×1000.

FIG. 14A-14B: Sequences of human and mouse heparanase.

FIG. 14A: Human heparanase (GenBank Accession No. AF144325.1; SEQ ID NO:57). Target sites for ribozyme Rz2, and siRNA-H1 and siRNA-H2 are indicated.

FIG. 14B: Mouse heparanase (GenBank Accession No. NM_{—}152803.2; SEQ ID NO:56). Target sites for Si1 and Si2 are indicated.

FIG. 15A-15B: Schematic representation of plasmids pSUPER and pLentiLox 3.7.

FIG. 15A: pSUPER.

FIG. 15B: pLentiLox 3.7.

### DETAILED DESCRIPTION OF THE INVENTION

As mentioned above, a number of evidence suggests that heparanase plays an important role in sustaining the pathology of malignant tumors. Interestingly, expression of the heparanase gene and protein correlate with invasive and metastatic potential of several malignant tumors, including bladder [Gohji (2001) id ibid.], colon [Friedmann (2000) id ibid.], gastric [Tang, W. et al. (2002) Mod. Pathol. 15:593-8], breast [Maxhimer, J. B. et al. (2002) Surgery 132:326-33], oral [Ikuta, M. et al. (2001) Oral Oncol. 37:177-84] oesophageal [Mikami, S. et al. (2001) J. Cancer Res. 92:1062-73], pancreatic [Koliopanos (2001) id ibid.; Kim, A W, et al. (2002) J. Gastrointest. Surg. 6:167-72; Rohloff, J. et al. (2002) Br. J. Cancer 86:1270-5] and brain [Marchetti, D. and Nicolson, G. L. (2001) Adv. Enzyme Regul. 41:343-59] carcinomas, as well as multiple myeloma [Kelly (2003) id ibid.] and acute myeloid leukaemia [Vlodavsky, I. et al. (2002) Semin. Cancer Biol. 12:121-9]. These results and the unexpected occurrence of a single functional heparanase indicate that this enzyme provides an attractive target for the development of anti-cancer therapy. As mentioned before, various polyanionic compounds, capable of inhibiting heparanase enzymatic activity, such as heparin, laminaran sulfate and maltohexose sulfate, exhibit anti-tumor and anti-metastatic effects. However, due to the multiple biological activities of these compounds, the mechanism of their anti-tumor activity and its causal relation to heparanase inhibition are not straightforward. Moreover, these molecules are difficult to be targeted to a specific tissue site, and their pleiotropic interactions with the ECM and cell surface might produce undesirable effects. Similarly, studies on the causal involvement of heparanase in cancer progression are hampered by the lack of effective neutralizing anti-heparanase antibodies. Recently, an attempt to utilize a more specific antisense approach has been reported [Uno, F. et al. (2001) Cancer Res. 61:7855-60], although the animal model used in that study is not typical for metastatic research, since the tumor cells are injected intrathoracically and hence do not encounter extracellular barriers to invade.

In the present invention, a hammerhead anti-heparanase (anti-hpa) ribozyme was designed and used for and created by the inventors, who demonstrated that ribozyme mediated inhibition of heparanase expression led to a marked decrease in invasive and adhesive abilities of mouse and human cancer cells in vitro, as well as their metastatic and angiogenic potentials in vivo. A highly specific anti-hpa siRNA that effectively silenced the heparanase gene was designed and a vector that enabled its stable expression in cancer cells was constructed by the inventors. The siRNA-mediated silencing of endogenous heparanase in mouse B16-BL6 melanoma cells resulted in an almost complete inhibition of melanoma cell invasion in vitro and lung colonization in vivo.

In addition, the present research was undertaken to further elucidate the source and biological significance of heparanase in inflammation, and the potential of gene-silencing technology to overcome the inflammatory condition. For that purposes, a DTH inflammatory model was applied, as well as a recently developed in vivo systems for heparanase overexpression (hpa-transgenic mice) [Zcharia, E. et al. (2004) Faseb J. 18:252-263], together with monitoring heparanase promoter activation [Elkin, M. et al. (2003) Cancer Res. 63:8821-8826; Zcharia, E. et al. (2005) Am. J. Pathol. 166:999-1008].

Endothelial cells are now recognized as active participants in DTH reactivity and other types of inflammatory processes [Black (1999) id ibid.; Sana, T. R. et al. (2005) Cytokine 29:256-269; Standage, B. A. et al. (1985) J. Cell Biochem. 29:45-56]. Following alterations induced by pro-inflammatory cytokines (i.e., TNF-α, IFN-γ) acting in concert, endothelial cells become activated and synthesize numerous adhesion molecules involved in leukocytes-endothelium interactions [Black (1999) id ibid.]. Endothelial cells are also capable of secreting different molecules (i.e., cytokines) which attract various types of immune cells into the site of inflammation and increase the mobility of adherent leukocytes from the peripheral blood. Moreover, endothelial cells were proposed to contribute to local vessel hyperpermeability by remodeling the subendothelial BM and thus allowing the extravasation of plasma macromolecules (e.g., fibrinogen) and immunocytes. However, attempts to identify the molecular mechanism responsible for increased vascular permeability in DTH inflammation were met with limited success. The data presented herein directly implicate the heparanase enzyme, locally expressed by the vascular endothelium at the site of inflammation, in degradation of the subendothelial BM and subsequent vascular leakage—a hallmark of delayed hypersensitivity skin reactions.

Thus, the inventors applied two powerful gene-silencing technologies (ribozyme and RNA interference), resulting in functional inactivation of the heparanase gene in diverse cellular and animal tumor and inflammation models. Ribozyme targeting led to a marked inhibition of in vitro invasive and adhesive potentials of cells that either naturally express elevated levels of the endogenous enzyme (i.e. MDA-435 breast carcinoma) [Vlodavsky (1999a) id ibid.], or genetically engineered to overexpress the human hpa gene (C6 glioma, Eb lymphoma) [Goldshmidt, O. et al. (2002) Proc. Natl. Acad. Sci. USA. 99:10031-6]. Even more impressive, the anti-hpa ribozyme significantly inhibited both the vascularization of cHpaEb primary tumor and its spontaneous liver dissemination, in vivo. These effects were reflected by an increased survival of nude mice inoculated with ribozyme-expressing cHpaEb lymphoma cells, as compared to mice inoculated with cells co-expressing the secreted enzyme and a control ribozyme. The biological and therapeutic relevance of the hpa-silencing approach was further validated, utilizing the highly metastatic B16-BL6 mouse melanoma cells [Vlodavsky (1994) id ibid.] transfected with mouse hpa specific siRNA. Knock-down of hpa expression resulted in an almost complete inhibition (˜77%) of lung colonization following intravenous inoculation of siRNA transfected B16-BL6 cells, as compared to cells transfected with the carrier plasmid alone.

The results of Example 6 reveal that induction of heparanase gene expression in the vascular endothelium is an important parameter for inflammatory response. Timely action of endothelial heparanase in the course of inflammation emerges as an essential step, allowing for remodeling of the vascular BM, increased vessel permeability, and extravasation of leukocytes and plasma proteins. A marked decrease in DTH was obtained upon local delivery of anti-heparanase siRNA. This present study represents the first successful application of anti-inflammatory therapy based on electroporation-assisted heparanase siRNA delivery in vivo. Given the critical role of heparanase in inflammation, tumor progression, and angiogenesis, the anti-heparanase siRNA delivery approach developed in this study is highly relevant to the design of future therapeutic interventions in these conditions.

Thus, the present invention relates to a nucleic acid molecule comprising at least one target specific sequence, which sequence is complementary to a target ribonucleotide sequence comprised within heparanase mRNA. The nucleic acid molecule of the invention is capable of inhibiting the expression of heparanase.

The term “nucleic acid molecule” refers to a polymer of nucleotides, or a polynucleotide, as described above. The term is used to designate a single molecule, or a collection of two or more molecules. Nucleic acids may be single stranded or double stranded, and may include coding regions and regions of various control elements and functional elements. “Polynucleotide” refers to a molecule comprised of two or more deoxyribonucleotides or ribonucleotides, or nucleotide analogs, preferably more than three, and usually more than ten. The exact size will depend on many factors, which in turn depends on the ultimate function or use of the nucleic acid molecule. It should be noted that the polynucleotide may be generated in any manner, including chemical synthesis, DNA replication, reverse transcription, or a combination thereof Preferably, the nucleic acid molecule of the invention is synthetic.

In addition, the nucleic acid molecule of the invention is preferably an isolated and purified molecule, as defined herein. The term “isolated” when used in relation to a nucleic acid, “an isolated nucleic acid molecule” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids, such as DNA and RNA, are found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs which encode a multitude of proteins. However, an isolated nucleic acid is in a chromosomal location different from that of in natural cells, or is otherwise flanked by a different nucleic acid sequence than that found in nature. The isolated nucleic acid molecule may be present in single-stranded or double-stranded form.

The term “purified” refers to molecules, such as nucleic acid sequences that are removed from their natural environment, isolated or separated. An “isolated nucleic acid sequence” is therefore a purified nucleic acid sequence. “Substantially purified” molecules are at least 60% free, preferably at least 75% free, and more preferably at least 90% free from other components with which they are naturally associated. As used herein, the term “purified” or “to purify” also refers to the removal of contaminants from a sample.

It should be noted that as used herein in the specification and in the claims section below, the term “heparanase” refers to an animal endoglycosidase which is specific for heparin or heparan sulfate proteoglyean substrates, as opposed to bacterial enzymes (heparinase I, II and III) which degrade heparin or heparan sulfate by means of β-elimination. Nonetheless, heparanase expression which is inhibited or neutralized according to the present invention can be of either recombinant or natural heparanase.

As indicated above, the nucleic acid molecule of the invention comprises a target specific sequence which is complementary to a sequence within heparanase RNA sequence. The terms “complementary” and “complementarity” refer to polynucleotides (i.e., a sequence of nucleotides) related by the base-pairing rules. For example, the sequence “A-G-T” is complementary to the sequence “T-C-A.” Complementarity may be “partial,” in which only some of the nucleic acid bases are matched according to the base pairing rules. Or, there may be “complete” or “total” complementarity between the nucleic acids. A complementary nucleic acid can form hydrogen bond(s) with another RNA sequence, such as the heparanase-derived sequence by either traditional Watson-Crick or other non-traditional types. A percent complementarity indicates the percentage of contiguous residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementarity). “Perfectly complementary” means that all the contiguous residues of a nucleic acid sequence will hydrogen bond with the same number of contiguous residues in a second nucleic acid sequence.

A target sequence is a sequence within heparanase whose expression is targeted for interference, inhibition, attenuation, disruption, augmentation, or other modulation. Preferably, the expression is targeted for interference. Most preferably the expression is targeted for attenuation.

The nucleic acid molecules of the invention are capable of inhibiting heparanase expression. As used herein in the specification and in the claims section below, the term “inhibit” and its derivatives refers to suppress or restrain from free expression. More particularly, “inhibition” when used in reference to gene expression or RNA function refers to a decrease in the level of gene expression or RNA function as the result of some interference with or interaction with gene expression or RNA function as compared to the level of expression or function in the absence of the interference or interaction. The inhibition by the nucleic acid molecules of the invention may be complete, in which there is no detectable expression or function, or it may be partial. Partial inhibition can range from near complete inhibition to near absence of inhibition; typically, inhibition is at least about 50% inhibition, or at least about 80% inhibition, or at least about 90% inhibition.

According to one preferred embodiment, the nucleic acid molecule of the invention may be a ribonucleic acid molecule having endonuclease activity.

By “a molecule having endonuclease activity” it is meant an RNA molecule which has complementarity in a target binding region to a specified gene target, for example heparanase, and also has an enzymatic activity which is active to specifically cleave target RNA. Said molecule is capable of catalyzing a series of reactions including the hydrolysis of phosphodiester bonds in trans (and thus can cleave other RNA molecules) under physiological conditions. Such enzymatic nucleic acid molecules can be targeted to virtually any RNA transcript, and achieve efficient cleavage in vitro. That is, the enzymatic RNA molecule is able to intermolecularly cleave RNA and thereby inactivate a target RNA molecule. The complementary regions allow sufficient hybridization of the enzymatic RNA molecule to the target RNA and which ensures specific cleavage. One hundred percent complementarity is preferred, but complementarity as low as 50-75% may also be useful in this invention. The nucleic acids may be modified at the base, sugar, and/or phosphate groups. The term enzymatic nucleic acid is used interchangeably with phrases such as ribozymes, catalytic RNA, enzymatic RNA, catalytic DNA, catalytic oligonucleotides, nucleozyme, DNAzyme, RNA enzyme, endoribonuclease, endonuclease, minizyme, leadzyme, oligozyme or DNA enzyme. All of these terminologies describe nucleic acid molecules with enzymatic activity. The specific enzymatic nucleic acid molecules described in the instant application are not meant to be limiting and those skilled in the art will recognize that all that is important in an enzymatic nucleic acid molecule of this invention is that it have a specific target binding site which is complementary to one or more of the target nucleic acid regions, and that it have nucleotide sequences within or surrounding that substrate binding site which impart a nucleic acid cleaving activity to the molecule.

Several basic varieties of naturally-occurring enzymatic RNAs are known presently. In general, enzymatic nucleic acids act by first binding to a target RNA. Such binding occurs through the target binding region of a enzymatic nucleic acid which is held in close proximity to an enzymatic region or catalytic region of the molecule that acts to cleave the target RNA. Thus, the enzymatic nucleic acid first recognizes and then binds a target mRNA through complementary base-pairing, and once bound to the correct site, acts enzymatically to cut the target RNA. Nucleic acid molecules having an endonuclease enzymatic activity are able to repeatedly cleave other separate RNA molecules in a nucleotide base sequence-specific manner. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets. Thus, a single ribozyme molecule is able to cleave many molecules of target RNA. In addition, the ribozyme is a highly specific inhibitor of gene expression, with the specificity of inhibition depending not only on the base-pairing mechanism of binding to the target RNA, but also on the mechanism of target RNA cleavage. Single mismatches, or base-substitutions, near the site of cleavage can completely eliminate catalytic activity of a ribozyme.

Therefore, the ribonucleic acid molecule having endonuclease activity of the invention is preferably a ribozyme, and more preferably a hammerhead ribozyme, which specifically cleaves heparanase RNA and thereby inhibits the expression of heparanase. Alternatively, the ribozyme of the invention is a hairpin ribozyme.

The enzymatic nature of a ribozyme is advantageous over other technologies, such as antisense technology (where a nucleic acid molecule simply binds to a nucleic acid target to block its translation) since the concentration of ribozyme necessary to affect a therapeutic treatment is lower than that of an antisense oligonucleotide. This advantage reflects the ability of the ribozyme to act enzymatically, since a single ribozyme molecule is able to cleave many molecules of target RNA.

In preferred embodiments of this invention, the enzymatic nucleic acid molecule is formed in a hammerhead or hairpin motif, but it should be noted that it may also be formed in the motif of a hepatitis delta virus, group I intron or RNaseP RNA (in association with an RNA guide sequence) or Neurospora VS RNA. Examples of such hammerhead, hairpin, hepatitis delta virus and RNase P motifs are described in the prior art [Scherer, L. J. and Rossi, J. J. (2003) Nature Biotech. 21(12): 1457-1465]. These specific motifs are not limiting in the invention and those skilled in the art will recognize that all that is important in an enzymatic nucleic acid molecule of the present invention is complementarity to one or more specific target RNA sequences within heparanase RNA, and that it have nucleotide sequences within or surrounding that substrate binding site which impart an RNA cleaving activity to the molecule.

The term “portion” when used in reference to a nucleic acid sequence (as in “a portion of a given sequence”) refers to fragments of that sequence. The fragments may range in size from four nucleotides to the entire nucleotide sequence minus one nucleotide.

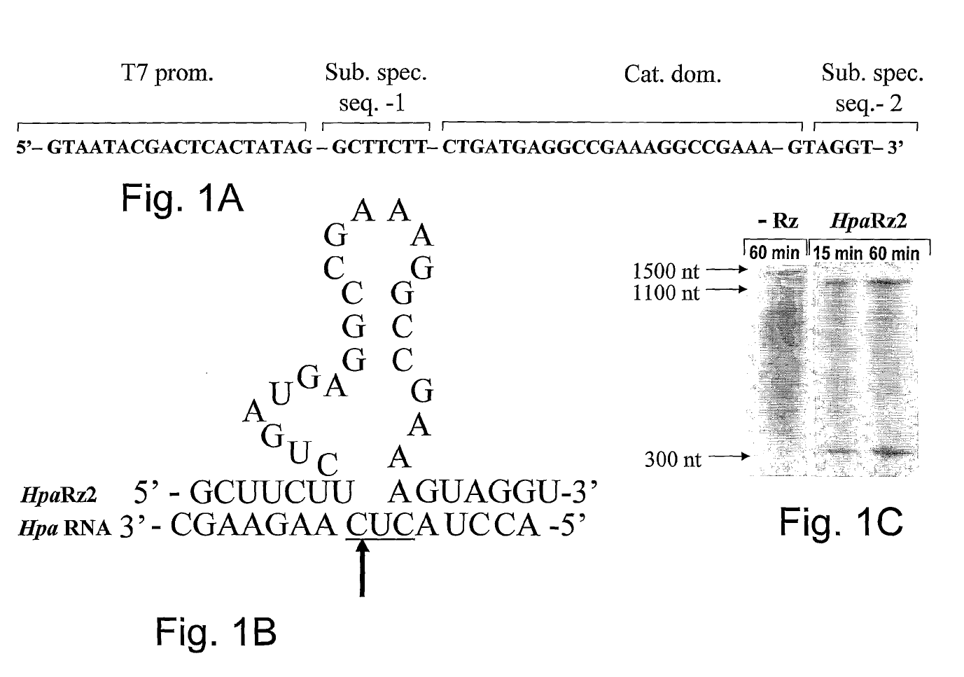

By “portion of a region which is complementary to a target RNA sequence” is meant that portion/region of a ribozyme which is complementary to (i.e., able to base-pair with) a portion of its target sequence within the heparanase RNA. Generally, such complementarity is 100%, but can be less if desired. For example, as few as 10 bases out of 14 may be base-paired. Such regions (preferably the first and third) are shown generally in FIG. 1A. That is, these regions contain sequences within a ribozyme which are intended to bring ribozyme and target RNA together through complementary base-pairing interactions. The ribozyme of the invention may have binding regions that are contiguous or non-contiguous and may be of varying lengths. The length of the binding arm(s) are preferably greater than or equal to four nucleotides and of sufficient length to stably interact with the target RNA; specifically 12-100 nucleotides; more specifically 14-24 nucleotides long. If two binding arms are chosen, the design is such that the length of the binding arms are symmetrical (i.e., each of the binding arms is of the same length; e.g., five and five nucleotides, six and six nucleotides or seven and seven nucleotides long) or asymmetrical (i.e., the binding arms are of different length; e.g., six and three nucleotides; three and six nucleotides long; four and five nucleotides long; four and six nucleotides long; four and seven nucleotides long; and the like).

By “catalytic domain” is meant that portion or region of the ribozyme essential for cleavage of a nucleic acid substrate (for example see FIG. 1A).

It should be noted that the heparanase sequences are preferably derived from a mammalian heparanase, preferably, human or mouse heparanase and most preferably, human heparanase.

In preferred embodiments of the present invention, a nucleic acid molecule, e.g., a ribozyme, is 10 to 100 nucleotides in length, e.g., in specific embodiments 35, 36, 37, or 38 nucleotides in length (e.g., for particular ribozymes). In particular embodiments, the nucleic acid molecule is 15-100, 17-100, 20-100, 21-100, 23-100, 25-100, 27-100, 30-100, 32-100, 35-100, 40-100, 50-100, 60-100, 70-100, or 80-100 nucleotides in length. Instead of 100 nucleotides being the upper limit on the length ranges specified above, the upper limit of the length range can be, for example, 30, 40, 50, 60, 70, or 80 nucleotides. Thus, for any of the length ranges, the length range for particular embodiments has lower limit as specified, with an upper limit as specified which is greater than the lower limit. For example, in a particular embodiment, the length range can be 35-50 nucleotides in length. All such ranges are expressly included. Also in particular embodiments, a nucleic acid molecule can have a length which is any of the lengths specified above, for example, 36 nucleotides in length.

A particular ribozyme of the invention comprises the ribonucleic acid sequence as denoted by SEQ ID NO: 19 or any analog, variant, derivative and fragment thereof. It should be noted that SEQ ID NO: 19, is complementary to nucleotides 589 to 603, of human heparanase cDNA sequence as denoted by GenBank Accession No. AF144325.1. Preferably, the ribozyme of the invention has the ribonucleic acid sequence as denoted by SEQ ID NO: 19, and is designated HpaRz2.

Synthesis of nucleic acids larger than 100 nucleotides in length is difficult and using automated methods and the therapeutic cost of such molecules is prohibitive. In this invention, small enzymatic nucleic acid motifs (e.g., of the hammerhead structure) may be used for exogenous delivery. The simple structure of these molecules increases the ability of the enzymatic nucleic acid to invade targeted regions of the mRNA structure. Unlike the situation where the hammerhead structure is included within longer transcripts, there is no non-enzymatic nucleic acid flanking sequences to interfere with correct folding of the enzymatic nucleic acid structure or with complementary regions.

Generally, RNA is synthesized and purified by methodologies based on: tetrazole to activate the RNA amidite, NH₄OH to remove the exocyclic amino protecting groups, tetra-n-butylammonium fluoride (TBAF) to remove the 2′-OH alkylsilyl protecting groups, and gel purification and analysis of the deprotected RNA. In particular this applies to certain class of RNA molecules, ribozymes. These may be formed either chemically or using enzymatic methods. Examples of the chemical synthesis, deprotection, purification and analysis procedures are provided by different references [Usman et al. (1987) J. Chem. Soc. 109:7845; Scaringe et al. (1990) Nucleic Acids Res. 18:5433-5341; Perreault et al. (1991) Biochemistry 30:4020-4025; Slim and Gait (1991) Nucleic Acids Res. 19:1183-1188; Odai et al. (1990) FEBS Lett. 267:150-152].

Alternatively, the ribonucleic acid molecule of the invention is a siRNA comprising a double strand ribonucleic acid (dsRNA) sequence, wherein at least a portion of one strand of said dsRNA comprises a sequence complementary to a target sequence within the heparanase mRNA sequence.

The term “siRNAs” refers to short interfering RNAs. The term “RNA interference” or “RNAi” refers to the silencing or decreasing of gene expression by siRNAs. It is the process of sequence-specific, post-transcriptional sequence-specific gene silencing in animals and plants, initiated by siRNA that is homologous in its duplex region to the sequence of the silenced gene. The gene may be endogenous or exogenous to the organism, integrated into a chromosome or present in a transfection vector which is not integrated into the genome. The expression of the gene is either completely or partially inhibited. RNAi may also inhibit the function of a target RNA, and said function may be completely or partially inhibited.

RNAi is a multistep process. In a first step there is cleavage of large dsRNAs, through the action of the Dicer enzyme (a RNase III endonuclease), into 21-23 ribonucleotides-long double stranded effector molecules called small interfering RNAs (siRNAs). These siRNAs duplexes then associate with an endonuclease-containing complex, known as RNA-induced silencing complex (RISC). The RISC specifically recognises and cleaves the endogenous mRNAs containing a sequence complementary to one of the siRNA strands. One of the strands of the double-stranded siRNA molecule comprises a nucleotide sequence that is complementary to a nucleotide sequence of the endogenous mammalian target gene, specifically heparanase or a portion thereof, and the second strand of the double-stranded siRNA molecule comprises a nucleotide sequence substantially similar to the nucleotide sequence of the endogenous mammalian target gene (heparanase) or a portion thereof.

In some embodiments, siRNAs comprise a duplex, or double-stranded region, of about 18-25 nucleotides long; often siRNAs contain from about two to four unpaired nucleotides at the 3′ end of each strand. At least a portion of one strand of the duplex or double-stranded region of a siRNA is substantially homologous to or substantially complementary to a target sequence within heparanase RNA molecule. The strand complementary to a target RNA molecule is the “antisense strand;” the strand homologous to the target RNA molecule is the “sense strand” (which is also complementary to the siRNA antisense strand). siRNAs may also contain additional sequences. Non-limiting examples of such sequences include linking sequences, or loops, as well as stem and other folded structures. siRNAs appear to function as key intermediaries in triggering RNA interference in invertebrates and in vertebrates, and in triggering sequence-specific RNA degradation during posttranscriptional gene silencing.

The term “dsRNA” as used herein refers to a siRNA molecule that comprises two separate unlinked strands of RNA which form a duplex structure, such that the siRNA molecule comprises two RNA polynucleotides.

The term “target sequence within heparanase RNA molecule” as used herein refers to a sequence within heparanase RNA molecule to which at least one strand (or any portion thereof of the short double-stranded region of the siRNA is homologous or complementary. Typically, when such homology or complementarity is about 100%, the siRNA or ribozyme is able to silence or inhibit expression of the target RNA molecule. Although it is believed that processed mRNA is a target of siRNA and ribozyme, the present invention is not limited to any particular hypothesis, and such hypotheses are not necessary to practice the present invention. Thus, it is contemplated that other heparanase RNA molecules may also be targets of siRNA or ribozyme, such as unprocessed mRNA of heparanase.

siRNAs are involved in RNA interference (as described above), where one strand of a duplex (the antisense strand) is complementary to a target gene RNA. The siRNA molecules described to date are a duplex of short, complementary strands. Such duplexes are usually prepared by separately chemically synthesizing the two separate complementary strands, and then combining them in such a way that the two separate strands form duplexes. Alternatively, siRNAs are made through processing of longer, double stranded RNAs through exposure to Drosophila embryo lysates or through an in vitro system derived from S2 cells. The duplex siRNAs are then used to transfect cells. Although there is much that remains unknown about the process of RNAi (such as the enzymes involved, as noted above), a recent report provides “rules” for the “rational” design of siRNAs which are the most potent siRNA duplexes [Elbashir et al. (2001b) EMBO J. 20(23):6877-6888]. These rules include that the siRNA duplexes be composed by a 21 nucleotide-long sense strand and a 21 nucleotide-long antisense strand selected to form a 19 base pair double helix with 3′ end overhangs two nucleotides long.

Target recognition is highly sequence-specific, but the 3′ most nucleotide of the guide (or antisense) siRNA does not contribute to the specificity of target recognition, whereas the penultimate nucleotide of the 3′ overhang affects target RNA cleavage. The 5′ end also appears more permissive for mismatched target RNA recognition when compared to the 3′ end. Nucleotides in the center of the siRNA, located opposite to the target RNA cleavage site, are important determinants, and even single nucleotide changes essentially abolish RNAi. Identical 3′ overhanging sequences are suggested to minimize sequence effects that may affect the ratio of sense- and anti-sense-targeting (and cleaving) siRNAs. Such rules, where applicable, may be useful in the design of the siRNAs of the present invention. Methods of chemical synthesis are diverse. Non-limiting examples are provided in the literature [for example in U.S. Pat. No. 5,889,136, U.S. Pat. No. 4,415,732 and U.S. Pat. No. 4,458,0661.

Further to the preparation, the duplex RNAs are then mixed with a transfection agent and added to cell culture at concentrations of about 100 nM. It is further recommended that the selection of the target sequence should be constrained so that they begin with AA and end with TT, so that the AA and TT overhang sequences may be fashioned from the target sequence itself. Moreover, the symmetric 3′ overhangs aid the formation of approximately equimolar ratios of sense and antisense target RNA-cleaving siRNAs.

It should be noted that also hairpin siRNAs, full or partial, are within the scope of the invention. The term “hairpin siRNA” refers to a siRNA molecule that comprises at least one duplex region where the strands of the duplex are connected or contiguous at one end, such that the siRNA molecule comprises a single RNA polynucleotide. The antisense sequence, or sequence which is complementary to a target sequence within heparanase RNA, is a part of the at least one double stranded region. The term “full hairpin siRNA” refers to a hairpin siRNA that comprises a duplex or double stranded region of about 18-25 base pairs long, where the two strands are joined at one end by a linking sequence, or loop. At least one strand of the duplex region is an antisense strand, and either strand of the duplex region may be the antisense strand. The region linking the strands of the duplex, also referred to as a loop, comprises at least three nucleotides. The sequence of the loop may also be a part of the antisense strand of the duplex region, and thus it is itself complementary to a target sequence within heparanase RNA molecule. The term “partial hairpin siRNA” refers to a hairpin siRNA which comprises an antisense sequence (or a region or strand complementary to a target sequence within heparanase RNA) of about 18-25 bases long, and which forms less than a full hairpin structure with the antisense sequence. In some embodiments, the antisense sequence itself forms a duplex structure of some or most of the antisense sequence. In other embodiments, the siRNA comprises at least one additional contiguous sequence or region, where at least part of the additional sequence(s) is complementary to part of the antisense sequence.

A dsRNA of the siRNA described by the invention may further comprise mismatch. The term “mismatch” when used in reference to siRNAs refers to the presence of a base in one strand of a duplex region of which at least one strand of an siRNA is a member, where the mismatched base does not pair with the corresponding base in the complementary strand, where pairing is determined by the general base-pairing rules. The term “mismatch” also refers to the presence of at least one additional base in one strand of a duplex region of which at least one strand of an siRNA is a member, where the mismatched base does not pair with any base in the complementary strand, or to a deletion of at least one base in one strand of a duplex region which results in at least one base of the complementary strand being without a base pair. A mismatch may be present in either the sense strand, or antisense strand, or both strands, of siRNA. If more than one mismatch is present in a duplex region, the mismatches may be immediately adjacent to each other, or they may be separated by from one to more than one nucleotide.

Thus, in some embodiments, a mismatch is the presence of a base in the antisense strand of an siRNA which does not pair with the corresponding base in the complementary strand of the target siRNA. In other embodiments, a mismatch is the presence of a base in the sense strand, when present, which does not pair with the corresponding base in the antisense strand of the siRNA. In yet other embodiments, a mismatch is the presence of a base in the antisense strand that does not pair with the corresponding base in the same antisense strand in a foldback hairpin siRNA.

Where the siRNAs of the invention comprise sequences complementary to target sequences derived from the mouse heparanase, said sequences correspond to the following: SEQ ID NO: 26 is complementary to nucleotides 425 to 443 of the mouse heparanase cDNA sequence, as denoted by GenBank Accession No. NM_{—}152803.2. SEQ ID NO: 27, is complementary to SEQ ID NO: 26, and therefore it is homologous to the mouse heparanase sequence. SEQ ID NO: 28 is complementary to nucleotides 484 to 502 of the mouse heparanase cDNA sequence, as denoted by GenBank Accession No. NM_{—}152803.2. SEQ ID NO: 29, is complementary to SEQ ID NO: 28, and therefore it is homologous to the mouse heparanase sequence.

Where the siRNAs of the invention comprise sequences complementary to target sequences derived from the human heparanase, said sequences correspond to the following: SEQ ID NO: 30 is complementary nucleotides 1034 to 1052 of the human heparanase cDNA sequence, as denoted by GenBank Accession No. AF 144325.1. SEQ ID NO: 31 is complementary to SEQ ID NO:30, and is therefore homologous to the human heparanase sequence. SEQ ID NO:32 is complementary to nucleotides 851 to 869 of the human heparanase cDNA sequence, as denoted by GenBank Accession No. AF 144325.1. SEQ ID NO: 33 is complementary to SEQ ID NO: 32, or any functional derivatives thereof, and is therefore homologous to the human heparanase sequence.

The terms derivatives and functional derivatives as used herein mean any nucleic acid molecule comprising the nucleic acid sequence of any one of SEQ ID NOs:19, 26, 27, 28, 29, 30, 31, 32 and 33 with any insertions, deletions, substitutions and modifications that do not interfere with said nucleic acid ability to inhibit heparanase expression (hereafter referred to as “derivative/s”). A derivative should maintain a minimal homology to said nucleic acid sequence, e.g. even less than 30%. It should be appreciated that by the term “insertions” as used herein is meant any addition of nucleotides to the nucleic acid molecules of the invention, between 1 to 50 nucleotides, preferably, between 20 to 1 nucleotides and most preferably, between 1 to 10 nucleotides.

It should be noted that the nucleic acid molecule of the invention may comprise more then one siRNA or ribozyme molecule, optionally, linked together by a linker or otherwise conjugated. The term “linker” when used in reference to a multiplex siRNA or ribozyme molecule refers to connecting means that joins two siRNA or ribozyme molecules. Such connecting means are typically though not necessarily a region of a nucleotide contiguous with a strand of each siRNA or ribozyme molecule, the region of contiguous nucleotide is referred to as a “joining sequence.”

It should be further noted that the siRNA of the invention may be formed from one or more strands of polymerized ribonucleotide. When formed of only one strand, it takes the form of a self-complementary hairpin-type or stem and loop structure that doubles back on itself to form a partial duplex. The self-duplexed portion of the RNA molecule may be referred to as the “stem” and the remaining, connecting single stranded portion referred to as the “loop” of the stem and loop structure. When made of two strands, they are substantially complementary.

The siRNA provided by the present invention allows for the modulation and especially the attenuation of heparanase expression when such a heparanase gene is present and liable to expression within a cell. Modulation of expression can be partial or complete inhibition of gene function, or even the up-regulation of other, secondary target genes or the enhancement of expression of such genes in response to the inhibition of the primary target gene. Attenuation of gene expression may include the partial or complete suppression or inhibition of gene function, transcript processing or translation of the transcript. In the context of RNA interference, as indicated above, modulation of gene expression is thought to proceed through a complex of proteins and RNA, specifically including small, dsRNA that may act as a “guide” RNA. The siRNA therefore is thought to be effective when its nucleotide sequence sufficiently corresponds to at least part of the nucleotide sequence of the target gene. Although the present invention is not limited by this mechanistic hypothesis, it is highly preferred that the sequence of nucleotides in the siRNA be substantially identical to at least a portion of the target heparanase sequence.

Any of the siRNAs of the invention must be designed so that they are specific and effective in suppressing the expression of heparanase. Methods of selecting the target sequences, i.e. sequences from heparanase whereto the siRNAs will guide the degradative machinery, are directed to avoiding sequences that may interfere with the siRNA's guide function while including sequences that are specific to the gene. Typically, siRNA target sequences of about 21 to 23 nucleotides in length are most effective. This length reflects the lengths of digestion products resulting from the processing of much longer RNAs as described above.

Several further modifications to siRNA sequences have been suggested in order to alter their stability or improve their effectiveness.

The following is a non-limiting list of possible modifications to be made to the siRNA that may result in higher potency through reduced stability of the siRNA duplex structure.

Phosphorothioates: Phosphorothioates reduce duplex stability approximately 0.5° C. to 1° C. per modification. They can be substituted at one or more nucleotide positions along the length of the siRNA.

Inosine: Substitution of inosine (I) for G's at one or more positions in the siRNA will reduce duplex stability and thereby enhance siRNA potency. I:C base pairs form only two hydrogen bonds (as opposed to three in G:C base-pairs), reducing the stability of the duplex.

Thio uridine: 4-thio uridine forms only a single hydrogen bond with adenosine and therefore the substitution of one or more uracils (U) in the siRNA results in duplex structures of reduced stability.

Ethyl cytosine: 4-ethyl cytosine forms only two hydrogen bonds with guanosine, reducing the stability of G:C base-pairs. Use of 4-ethyl cytosine at one or more positions in the siRNA is expected to reduce stability of the duplex structure.

Nitropyrrole nucleoside and 5-nitroindole nucleoside (5-nitroindole): Both of these nucleosides hybridize to all four natural nucleosides, but with lower affinity than canonical base-pairs. Thus, substitution of an appropriate number of nucleotides of the siRNA with these nucleosides will result in reduced overall duplex stability without loss of appropriate sequence specificity. The selection of the appropriate number and position of such nucleoside substitutions are well within the skill of the ordinary artisan.

Abasic sites: There are several nucleotide linkers that do not have an associated base. These can be introduced at one or more sites in the sense strand of the siRNA to eliminate one or more base-pairs and reduce the stability of the siRNA duplex. Nucleic acid helices with abasic sites have reduced melting temperatures, i.e. reduced duplex stability.

The nucleic acid molecules of the invention may be introduced into cells directly, or can be complexed with cationic lipids, packaged within liposomes, or otherwise delivered to target cells or tissues, in a number of ways. Preferred embodiments include micro-injection, bombardment by particles covered by the siRNA or ribozyme, soaking the cell or organism in a solution of the siRNA or ribozyme, electroporation of cell membranes in the presence of siRNA or ribozyme, liposome-mediated delivery of siRNA or ribozyme and transfection mediated by chemicals such as polyamines, calcium phosphate, viral infection, transformation, and the like. In further preferred embodiments, siRNA or ribozyme is introduced along with components that enhance RNA uptake by the cell, stabilize the annealed strands, or otherwise increase inhibition of the target gene. In a most preferred embodiment, cells are conveniently incubated in a solution containing the siRNA or ribozyme.

In another aspect of the invention, ribozymes that cleave target RNA molecules or siRNA, which indirectly leads to cleavage and inhibiting heparanase expression and activity, are expressed from transcription units inserted into DNA or RNA vectors. The recombinant vectors are preferably DNA plasmids or viral vectors. Ribozyme or siRNA expressing viral vectors could be constructed based on, but not limited to, adeno-associated virus, retrovirus, adenovirus, lenti-virus or alphavirus. Preferably, the recombinant vectors capable of expressing the ribozymes or siRNA are delivered as described above, and persist in target cells. Alternatively, viral vectors may be used that provide for transient expression of ribozymes or siRNA. Such vectors might be repeatedly administered as necessary. Once expressed, the ribozymes cleave the target mRNA and the siRNA leads to such cleavage. Delivery of ribozyme or siRNA expressing vectors could be systemic, such as by intravenous or intramuscular administration, by administration to target cells ex-planted from the patient followed by reintroduction into the patient, or by any other means that would allow for introduction into the desired target cell.

“Vector”, as used herein, encompases vectors such as plasmids, viruses, bacteriophage, integratable DNA fragments, and other vehicles, which enable the integration of DNA fragments into the genome of the host.

Expression vectors are typically self-replicating DNA or RNA constructs containing the desired gene or its fragments, and operably linked genetic control elements that are recognized in a suitable host cell and effect expression of the desired genes. These control elements are capable of effecting expression within a suitable host. Generally, the genetic control elements can include a prokaryotic promoter system or a eukaryotic promoter expression control system. This typically includes a transcriptional promoter, an optional operator to control the onset of transcription, transcription enhancers to elevate the level of RNA expression, RNA splice junctions, sequences that terminate transcription and so forth. Expression vectors usually contain an origin of replication that allows the vector to replicate independently of the host cell.

A vector may additionally include appropriate restriction sites, antibiotic resistance or other markers for selection of vector-containing cells. Plasmids are the most commonly used form of vector but other forms of vectors which serve an equivalent function and which are, or become, known in the art are suitable for use herein.

The terms “in operable combination”, “in operable order” and “operably linked” refer to the linkage of nucleic acid sequences in such a manner that a nucleic acid molecule capable of directing the transcription of a given gene.

The term “regulatory element” refers to a genetic element that controls some aspect of the expression of nucleic acid sequences. For example, a promoter is a regulatory element that facilitates the initiation of transcription of an operably linked coding region. Other regulatory elements are splicing signals, polyadenylation signals, termination signals, etc.

The terms “promoter element,” “promoter,” or “promoter sequence” as used herein, refer to a DNA sequence that is usually located at the 5′ end of the protein coding region. The promoter functions as a switch, activating the expression of a gene. Promoters may be tissue specific or cell specific.

Thus, the term “expression vector” as used herein, refers to a vector comprising one or more expression cassettes. Such expression cassettes include those of the present invention, where expression results in an siRNA or ribozyme transcript.

Preferred vectors used by the invention as demonstrated by the following Examples include the pcDNA3, which was used for expression of the ribozymes of the invention. Other specifically preferred examples for suitable vectors may be the pSUPER and the pLentiLOX 3.7, that were used for siRNA construction, as described in the Examples.

Therefore, preferred expression vectors of the invention are pCDNA3-HpaRz2, encoding a specific ribozyme of the invention, and pSUPER-s1, pSUPER-s2, pSUPER-H1, pSUPER-H2, pLentiLOX 3.7-S1, pLentiLOX 3.7-S2, pLentiLOX 3.7-H1, and pLentiLOX 3.7-H2, encoding preferred siRNA molecules of the invention, as demonstrated in the Examples.

The invention also provides a host cell transformed or transfected with any of the expression vectors of the invention. Suitable host cells include prokaryotes, lower eukaryotes, and higher eukaryotes. Prokaryotes include gram negative and gram positive organisms, e.g., E. coli and B. subtilis. Lower eukaryotes include yeast, S. cerevisiae and Pichia, and species of the genus Dictyostelium. Higher eukaryotes include established tissue culture cell lines from animal cells, both of non-mammalian origin, e.g., insect cells and birds, and of mammalian origin, e.g., human and other primate, and of rodent origin.

“Cells”, “host cells” or “recombinant cells” are terms used interchangeably herein. “Host cell” as used herein refers to cells which can be recombinantly transformed or transfected with vectors constructed using recombinant DNA techniques. A drug resistance or other selectable marker is intended in part to facilitate the selection of the transformants. Additionally, the presence of a selectable marker, such as drug resistance marker may be of use in keeping contaminating microorganisms from multiplying in the culture medium. Such a pure culture of the transformed host cell would be obtained by culturing the cells under conditions which require the induced phenotype for survival. It is understood that such terms refer not only to the particular subject cells but to the progeny or potential progeny of such a cell.

The term “transfection” refers to the introduction of foreign DNA into cells. Transfection may be accomplished by a variety of means known to the art including calcium phosphate-DNA co-precipitation, DEAE-dextran-mediated transfection, polybrene-mediated transfection, glass beads, electroporation, microinjection, liposome fusion, lipofection, protoplast fusion, bacterial infection, viral infection, biolistics (i.e., particle bombardment) and the like. The terms “transfect” and “transform” (and grammatical equivalents, such as “transfected” and “transformed”) are used interchangeably. The term “stable transfection” or “stably transfected” refers to the introduction and integration of foreign DNA into the genome of the transfected cell. The term “stable transfectant” refers to a cell that has stably integrated foreign DNA into the genomic DNA. The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells that have taken up foreign DNA but have failed to integrate this DNA.

The terms “infecting” and “infection” when used with a bacterium refer to co-incubation of a target biological sample, (e.g., cell, tissue, etc.) with the bacterium under conditions such that nucleic acid sequences contained within the bacterium are introduced into one or more cells of the target biological sample.

The data described in the present application demonstrate for the first time successful application of ribozyme- and siRNA-mediated gene silencing to effectively reduce the levels of heparanase. The results of the present invention clearly highlight the decisive role of heparanase in tumor angiogenesis, growth and metastasis, as well as in inflammation. Apart from the potential promise for cancer treatment, the specific heparanase gene silencing tools applied in the present study, are expected to better clarify the molecular and cellular mechanisms underlying some of the recently described heparanase-mediated processes such as cell adhesion and survival signals in vitro, as well as tissue repair, hair growth and bone formation in vivo. These tools, acting on the RNA level, are especially important in light of the recently discovered non-enzymatic functions of heparanase, which are not sensitive to the currently available heparanase inhibitors, which are specific to its enzymatic activity [Miao (1999) id ibid.; Parish (1999) id ibid.; Goldshmidt, O. et al. (2003) Faseb. J. 17:1015-25].

Therefore, in a third aspect the invention relates to a composition for the inhibition of heparanase expression, comprising as an active ingredient at least one isolated and purified nucleic acid molecule comprising at least one target specific sequence, which sequence is complementary to a target ribonucleotide sequence comprised within heparanase mRNA. The composition of the invention optionally further comprises a pharmaceutically acceptable carrier, diluent, excipient and/or additive.

As indicated herein before, any composition of the invention may comprise a multi-siRNA or ribozyme molecule comprising more than one siRNA or ribozyme molecules, mixed, conjugated or linked by a linker.

The pharmaceutical composition of the invention is intended for the treatment or the inhibition of a process or a pathologic disorder associated with heparanase over-expression.

The term “overexpression” refers to the production of a gene product, specifically, heparanase, in an organism or a certain tissue that exceeds levels of production in normal organisms or tissues. More specifically, “overexpression”, “overexpressing” and grammatical equivalents are used in reference to levels of mRNA to indicate a level of expression approximately at least 3-fold higher than that typically observed in a given tissue in a control organism. Levels of mRNA are measured using any of a number of techniques known to those skilled in the art including, like for example, but not limited to, Northern blot analysis.

The composition of the invention may comprise the active substance in free form and be administered directly to the subject to be treated. Alternatively, depending on the size of the active molecule, it may be desirable to conjugate it to a carrier prior to administration. Therapeutic formulations may be administered in any conventional dosage formulation. Formulations typically comprise at least one active ingredient, as defined above, together with one or more acceptable carriers thereof.

Each carrier should be both pharmaceutically and physiologically acceptable in the sense of being compatible with the other ingredients and not injurious to the patient. Formulations include those suitable for oral, rectal, nasal, or parenteral (including subcutaneous, intramuscular, intraperitoneal (IP), intravenous (IV) and intradermal administration. The formulations may conveniently be presented in unit dosage form and may be prepared by any methods well known in the art of pharmacy. The nature, availability and sources, and the administration of all such compounds including the effective amounts necessary to produce desirable effects in a subject are well known in the art and need not be further described herein.

The preparation of pharmaceutical compositions is well known in the art and has been described in many articles and textbooks, see e.g., Remington's Pharmaceutical Sciences, Gennaro A. R. ed., Mack Publishing Co., Easton, Pa., 1990, and especially pp. 1521-1712 therein.

More specifically, the nucleic acid molecule of the invention or a composition comprising the same, having heparanase inhibitory activity, may be administered by a route selected from oral, intravenous, parenteral, transdermal, subcutaneous, intravaginal, intranasal, mucosal, sublingual, topical and rectal administration and any combinations thereof.

The pharmaceutical forms suitable for injection use include sterile aqueous solutions or dispersions and sterile powders for the extemporaneous preparation of sterile injectable solutions or dispersions. In all cases the form must be sterile and must be fluid to the extent that easy syringeability exists. It must be stable under the conditions of manufacture and storage and must be preserved against the contaminating action of microorganisms, such as bacteria and fungi.

Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

In the case of sterile powders for the preparation of the sterile injectable solutions, the preferred method of preparation are vacuum-drying and freeze drying techniques which yield a powder of the active ingredient plus any additional desired ingredient from a previously sterile-filtered solution thereof

It should be noted that these are applicable for any composition described by the present invention.

As used herein, in the specification and in the claims section below, the term “treat” or treating and their derivatives includes substantially inhibiting, slowing or reversing the progression of a condition, substantially ameliorating clinical symptoms of a condition or substantially preventing the appearance of clinical symptoms of a condition.

As used herein, in the specification and in the claims section below, the phrase “associated with heparanase expression or catalytic activity” refers to conditions which at least partly depend on the expression or the catalytic activity of heparanase. It is understood that the expression or catalytic activity of heparanase under many such conditions can be normal, yet inhibition thereof in such conditions will result in improvement of the affected individual.

It should be further noted that the disorders or the conditions can be related to altered function of a HS-associated biological effector molecule, such as, but not limited to, growth factors, chemokines, cytokines and degradative enzymes. The condition can be, or involve, angiogenesis, tumor cell proliferation, invasion of circulating tumor cells, metastases, inflammatory disorders, autoimmune conditions and/or a kidney disorder.

The heparanase inhibitors (i.e., the nucleic acid molecules described herein) of the present invention may be used therefore for the treatment of diseases and disorders caused by or associated with heparanase expression or catalytic activity.

Involvement of heparanase in tumor angiogenesis has been correlated with the ability to release bFGF (FGF-2) and other growth factors from its storage within the ECM (extracellular matrix). These growth factors provide a mechanism for induction of neo-vascularization in normal and pathological situations.

Heparanase may thus facilitate not only tumor cell invasion and metastasis but also tumor angiogenesis, both critical steps in tumor progression.

It is therefore to be understood that the compositions and methods of the invention are useful for treating or inhibiting tumors at all stages, namely tumor formation, primary tumors, tumor progression and tumor metastasis.

Thus, in one embodiment of the present invention, the compositions and the methods of the invention can be used for inhibition of angiogenesis, and are thus useful for the treatment of diseases and disorders associated with angiogenesis or neovascularization such as, but not limited to, tumor angiogenesis, opthalmologic disorders such as diabetic retinopathy and macular degeneration, particularly age-related macular degeneration, and reperfusion of gastric ulcer.

As used herein to describe the present invention, “malignant proliferative disorder” “cancer”, “tumor” and “malignancy” all relate equivalently to a hyperplasia of a tissue or organ. If the tissue is a part of the lymphatic or immune systems, malignant cells may include non-solid tumors of circulating cells. Malignancies of other tissues or organs may produce solid tumors. In general, the compositions as well as the methods of the present invention may be used in the treatment of non-solid and solid tumors, for example, carcinoma, melanoma, leukemia, and lymphoma.

Therefore, according to a preferred embodiment, the peptide of the invention or a composition comprising the same, can be used for the treatment or inhibition of non-solid cancers, e.g. hematopoietic malignancies such as all types of leukemia, e.g., acute lymphocytic leukemia (ALL), acute myelogenous leukemia (AML), chronic lymphocytic leukemia (CLL), chronic myelogenous leukemia (CML), myelodysplastic syndrome (MDS), mast cell leukemia, hairy cell leukemia, Hodgkin's disease, non-Hodgkin's lymphomas, Burkitt's lymphoma and multiple myeloma, as well as for the treatment or inhibition of solid tumors such as tumors in lip and oral cavity, pharynx, larynx, paranasal sinuses, major salivary glands, thyroid gland, esophagus, stomach, small intestine, colon, colorectum, anal canal, liver, gallbladder, extraliepatic bile ducts, ampulla of vater, exocrine pancreas, lung, pleural mesothelioma, bone, soft tissue sarcoma, carcinoma and malignant melanoma of the skin, breast, vulva, vagina, cervix uteri, corpus uteri, ovary, fallopian tube, gestational trophoblastic tumors, penis, prostate, testis, kidney, renal pelvis, ureter, urinary bladder, urethra, carcinoma of the eyelid, carcinoma of the conjunctiva, malignant melanoma of the conjunctiva, malignant melanoma of the uvea, retinoblastoma, carcinoma of the lacrimal gland, sarcoma of the orbit, brain, spinal cord, vascular system, hemangiosarcoma and Kaposi's sarcoma.

The nucleic acid molecules of the invention, the expression vectors, the host cells or any compositions thereof, may be also useful for inhibiting or treating other cell proliferative diseases or disorders such as psoriasis, hypertrophic scars, acne and sclerosis/scleroderma, and for inhibition or treatment of other diseases or disorders such as polyps, multiple exostosis, hereditary exostosis, retrolental fibroplasia, hemangioma, and arteriovenous malformation.

Heparanase expression and catalytic activity correlates with the ability of activated cells of the immune system to leave the circulation and elicit both inflammatory and autoimmune responses. Interaction of platelets, granulocytes, T and B lymphocytes, macrophages and mast cells with the subendothelial ECM is associated with degradation of heparan sulfate (HS) by heparanase catalytic activity [Vlodavsky (1992) id ibid.]. The enzyme is released from intracellular compartments (e.g., lysosomes, specific granules) in response to various activation signals (e.g., thrombin, calcium ionophore, immune complexes, antigens, mitogens), suggesting its regulated involvement and presence in inflammatory sites and autoimmune lesions. Heparan sulfate degrading enzymes released by platelets and macrophages are likely to be present in atherosclerotic lesions [Campbell, K. H. et al. Exp. Cell Res. 200:156-167 (1992)]. Treatment of experimental animals with heparanase alternative substrates (e.g., non-anticoagulant species of low molecular weight heparin) markedly reduced the incidence of experimental autoimmune encephalomyelitis (EAE), adjuvant arthritis and graft rejection [Vlodavsky (1992) id ibid.; Lider, O. et al., J. Clin. Invest. 83:752-756 (1989)] in experimental animals, indicating that heparanase inhibitors may be applied to inhibit autoimmune and inflammatory diseases.

Therefore, in a further embodiment, the compositions and the methods of the invention may be useful for treatment of or amelioration of inflammatory symptoms in any disease, condition or disorder where immune and/or inflammation suppression is beneficial such as, but not limited to, treatment of or amelioration of inflammatory symptoms in the joints, musculoskeletal and connective tissue disorders, or of inflammatory symptoms associated with hypersensitivity, allergic reactions, asthma, atherosclerosis, otitis and other otorhinolaryngological diseases, dermatitis and other skin diseases, posterior and anterior uveitis, conjunctivitis, optic neuritis, scleritis and other immune and/or inflammatory ophthalmic diseases.

The inventors demonstrated the induction of locally-expressed heparanase at the site of inflammation in vivo and established its mechanistic involvement in DTH inflammatory reaction. Over-expression of heparanase in a mouse transgenic model significantly enhanced DTH reactivity. By monitoring in vivo activity of luciferase driven by the heparanase gene regulatory sequence, the inventors demonstrated that heparanase promoter activation occurs in the inflammation site upon the onset of a DTH response. Moreover, the present results showed that endothelial cells are the primary source of heparanase at the early stages of DTH inflammation. Furthermore, the present study shows that treatment with IFN-□, the key mediator of DTH inflammation [Black (1999) id ibid.; Muller, K. M. et al. (1993) J. Immunol. 150:5576-5584; Gautam, S. et al. (1994) J. Leukoc. Biol. 55:452-460; Mbow, M. L. et al. (1994) Cell Immunol. 156:254-261; Buchanan, K. L. and J. W. Murphy (1994) Infect. Immun. 62:2930-2939; Issekutz, T. B. et al. (1988) J. Immunol. 140:2989-2993], upregulates heparanase gene expression and increases heparanase enzymatic activity in cultured endothelial cells. Computerized analysis of the heparanase gene 1.8-kb regulatory sequence using MatInspector software [Quandt, K. et al. (1995) Nucleic Acids Res. 23:4878-4884] revealed two interferon-stimulated response elements (ISREs)—consensus sequences in the promoter region that specifically bind transcriptional factors activated by interferon (not shown). A more refined analysis of heparanase regulatory sequence will enable to locate the precise binding site(s) in the heparanase promoter responsible for the IFN-γ-induced transcription. Interestingly, TNF-α, another main inducer of local DTH responses Black (1999) id ibid.] has also been found to increase heparanase levels in endothelial cells [Chen (2004) id ibid. and the present study], in agreement with the previously reported ability of TNF-α to augment ECM degradation by endothelial cells [Bartlett (1995) id ibid.].

In another preferred embodiment, the compositions and the methods of the invention are useful for treatment or amelioration of an autoimmune disease such as, but not limited to, Eaton-Lambert syndrome, Goodpasture's syndrome, Greave's disease, Guillain-Barre syndrome, autoimmune hemolytic anemia (AIHA), hepatitis, insulin-dependent diabetes mellitus (IDDM), systemic lupus erythernatosus (SLE), multiple sclerosis (MS), myasthenia gravis (MG), plexus disorders e.g. acute bracllial neuritis, polyglandular deficiency syndrome, primary biliary cirrhosis, rheumatoid arthritis, scleroderma, thrombocytopenia, thyroiditis e.g. Hashimoto's disease, Sjogren's syndrome, allergic purpura, psoriasis, mixed connective tissue disease, polymyositis, dermatomyositis, vasculitis, polyarteritis nodosa, polymyalgia rheumatica, Wegener's granulomatosis, Reiter's syndrome, Beheet's syndrome, ankylosing spondylitis, pemphigus, bullous pernphigoid, dennatitis herpetiformis, insulin dependent diabetes, inflammatory bowel disease, ulcerative colitis and Crohn's disease.

Particularly, the compositions and methods of the invention are useful for the treatment of DTH.

Local in vivo electroporation of anti-heparanase siRNA into the ear skin markedly inhibited DTH reactivity, demonstrating the decisive involvement of heparanase in inflammation and the potent effect of siRNA in the treatment of DTH. In order to distinguish between heparanase expressed by local cellular elements at the site of inflammation vs. the enzyme expressed by circulating immunocytes, the in vivo experiments described herein were designed to achieve heparanase silencing one day prior to challenge with the hapten. Since T cells, known to mediate DTH response, attach to the vascular endothelium and extravasate toward the hapten only after the challenge [Abbas (2005) id ibid.], T cells were not exposed to anti-heparanase siRNA administered by local electroporation executed prior to challenge in the present study. The same is correct for any other free circulating cells of the immune system. On the other hand, endothelial cells are present at the future challenge site even before application of the hapten. Thus, siRNA application prior to challenge restricted the heparanase silencing to the local (e.g., endothelium), rather than circulating (e.g., T lymphocytes) cellular compartment. This approach allowed to specifically analyze the role of non-lymphocyte derived heparanase in inflammation. The decrease in heparanase protein, observed in the endothelium of immunostained ear tissue derived from pSi2-treated ears (FIG. 12C) demonstrated the effectiveness of heparanase silencing in vivo. The decrease in heparanase protein levels in pSi2-treated ear tissue correlated with preservation of the subendothelilal BM surrounding the capillary wall (FIG. 13B right) and absence of vessel leakage (FIG. 13A), as compared to control pSUPER-treated ears, in which capillary BM disruption, vessel hyperpermeability and ear swelling were clearly noted. In summary, induction of locally-expressed heparanase emerges as an important step in the series of events involved in onset of the inflammatory process. The present results suggest that upon hapten challenge, induction of endothelial heparanase expression driven by inflammatory cytokines (IFN-γ, TNF-α is responsible for subendothelial BM disintegration and subsequent plasma and immunocyte extravasation, resulting in development of a delayed type hypersensitivity reaction.

Heparanase has been proposed to be involved in the pathogenesis of proteinuria by selectively degrading the negatively charged side chains of heparan sulfate proteoglycans within the glomerular basement membrane. A loss of negatively charged heparan sulfate proteoglycans may result in alteration of the permselective properties of the glomerular basement membrane, loss of glomerular epithelial and endothelial cell anchor points, and liberation of growth factors and potentially leading to different kidney disorders, such as, passive Heymann nephritis (PHN), and puromycin aminonucleoside nephrosis (PAN).

Therefore, in another preferred embodiment, the compositions and methods of the invention are useful for treatment of or amelioration of any kidney disorder.

The magnitude of therapeutic dose of the composition of the invention will of course vary with the group of patients (age, sex, etc.), the nature of the condition to be treated and with the route administration and will be determined by the attending physician.

Although the method of the invention is particularly intended for the treatment of disorders associated with heparanase catalytic activity in humans, other mammals are included. By way of non-limiting examples, mammalian subjects include monkeys, equines, cattle, canines, and felines, rodents such as mice and rats, and pigs.

The pharmaceutical composition of the invention can be administered and dosed by the methods of the invention, in accordance with good medical practice, systemically, for example by parenteral, e.g. intravenous, intraperitoneal or intramuscular injection. In another example, the pharmaceutical composition can be introduced to a site by any suitable route including intravenous, subcutaneous, transcutaneous, topical, intramuscular, intraarticular, subconjunctival, or mucosal, e.g. oral, intranasal, or intraocular administration.

Local administration to the area in need of treatment may be achieved by, for example, local infusion during surgery, topical application, direct injection into the inflamed joint, directly onto the eye, etc.

For oral administration, the pharmaceutical preparation may be in liquid form, for example, solutions, syrups or suspensions, or in solid form as tablets, capsules and the like. For administration by inhalation, the compositions are conveniently delivered in the form of drops or aerosol sprays. For administration by injection, the formulations may be presented in unit dosage form, e.g. in ampoules or in multidose containers with an added preservative.

The compositions of the invention can also be delivered in a vesicle, for example, in liposomes. In another embodiment, the compositions can be delivered in a controlled release system.

As mentioned, the amount of the therapeutic or pharmaceutical composition of the invention which is effective in the treatment of a particular disease, condition or disorder will depend on the nature of the disease, condition or disorder and can be determined by standard clinical techniques. In addition, in vitro assays as well in viuo experiments may optionally be employed to help identify optimal dosage ranges. The precise dose to be employed in the formulation will also depend on the route of administration, and the seriousness of the disease, condition or disorder, and should be decided according to the judgment of the practitioner and each patient's circumstances. Effective doses may be extrapolated from dose-response curves derived from in vitro or animal model test systems.

As used herein, “effective amount” means an amount necessary to achieve a selected result. For example, an effective amount of the composition of the invention useful for inhibition of heparanase expression and thereby for the treatment of said pathology. These should be applicable for any method disclosed by the present application.

A number of methods of the art of molecular biology are not detailed herein, as they are well known to the person of skill in the art. Such methods include site-directed mutagenesis, PCR cloning, expression of cDNAs, analysis of recombinant proteins or peptides, transformation of bacterial and yeast cells, transfection of mammalian cells, and the like. Textbooks describing such methods are e.g., Sambrook et al. (1989) Molecular Cloning: A Laboratory Manual, Cold Spring Harbor Laboratory; ISBN: 0879693096; F. M. Ausubel (1988) Current Protocols in Molecular Biology, John Wiley & Sons, Inc., ISBN: 047150338X; and F. M. Ausubel et al., eds. (1995) Short Protocols in Molecular Biology, 3rd ed. John Wiley & Sons, ISBN: 0471137812. These publications are incorporated herein in their entirety by reference. Furthermore, a number of immunological techniques are not in each instance described herein in detail, as they are well known to the person of skill in the art. See e.g., Coligan et al., eds. (1997) Current Protocols in Immunology, John Wiley & Sons Inc., New York, N.Y.

Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the intended scope of the invention.

### EXAMPLES

Experimental Procedures

Sequences

Table 1 summarizes all sequences disclosed in the present specification.

TABLE 1
Sequences SEQ ID NO: 1-57

| Sequence | Description |
|---|---|
| SEQ ID NO: 1 | 5′ primer for T7 polymerase |
| SEQ ID NO: 2 | 3′ primer for T7 polymerase |
| SEQ ID NO: 3 | 5′ primer for preparation of HpaRz2 ribozyme |
| SEQ ID NO: 4 | 3′ primer for preparation of HpaRz2 ribozyme |
| SEQ ID NO: 5 | 5′ primer for preparation of control ribozyme |
| SEQ ID NO: 6 | 3′ primer for preparation of control ribozyme |
| SEQ ID NO: 7 | 5′ primer for preparation of mouse siRNA si1 |
| SEQ ID NO: 8 | 3′ primer for preparation of mouse siRNA si1 |
| SEQ ID NO: 9 | 5′ primer for preparation of mouse siRNA si2 |
| SEQ ID NO: 10 | 3′ primer for preparation of mouse siRNA si2 |
| SEQ ID NO: 11 | HPU-355 primer |
| SEQ ID NO: 12 | HPL-229 primer |
| SEQ ID NO: 13 | 431-U primer |
| SEQ ID NO: 14 | 876-L primer |
| SEQ ID NO: 15 | GAPDH-S primer |
| SEQ ID NO: 16 | GAPDH-AS primer |
| SEQ ID NO: 17 | L-19-U primer |
| SEQ ID NO: 18 | L-19-L primer |
| SEQ ID NO: 19 | HpaRz2 |
| SEQ ID NO: 20 | HpaRz1 |
| SEQ ID NO: 21 | HpaRz3 |
| SEQ ID NO: 22 | HpaRz4 |
| SEQ ID NO: 23 | HpaRz5 |
| SEQ ID NO: 24 | HpaRz6 |
| SEQ ID NO: 25 | Control ribozyme pContRz |
| SEQ ID NO: 26 | Mouse siRNA si1 (5′) |
| SEQ ID NO: 27 | Mouse siRNA si1 (3′) |
| SEQ ID NO: 28 | Mouse siRNA si2 (5′) |
| SEQ ID NO: 29 | Mouse siRNA si2 (3′) |
| SEQ ID NO: 30 | Human siRNA H1 (5′) |
| SEQ ID NO: 31 | Human siRNA H1 (3′) |
| SEQ ID NO: 32 | Human siRNA H2 (5′) |
| SEQ ID NO: 33 | Human siRNA H2 (3′) |
| SEQ ID NO: 34 | HpaRz1 template |
| SEQ ID NO: 35 | HpaRz2 template |
| SEQ ID NO: 36 | HpaRz3 template |
| SEQ ID NO: 37 | HpaRz4 template |
| SEQ ID NO: 38 | HpaRz5 template |
| SEQ ID NO: 39 | HpaRz6 template |
| SEQ ID NO: 40 | Ribozyme substrate 1477 bp |
| SEQ ID NO: 41 | 5′ primer for generating the anti-hpa |
| | siRNA pSi2-lenti |
| SEQ ID NO: 42 | 3′ primer for generating the anti-hpa |
| | siRNA pSi2-lenti |
| SEQ ID NO: 43 | Peptide used as an antigen for raising |
| | anti-heparanase antibody |
| SEQ ID NO: 44 | Nucleotides 586-600 of human heparanase |
| SEQ ID NO: 45 | Nucleotides 589-603 of human heparanase |
| SEQ ID NO: 46 | Nucleotides 729-743 of human heparanase |
| SEQ ID NO: 47 | Nucleotides 881-895 of human heparanase |
| SEQ ID NO: 48 | Nucleotides 883-897 of human heparanase |
| SEQ ID NO: 49 | Nucleotides 1194-1208 of human heparanase |
| SEQ ID NO: 50 | Sequence of oligonucleotide for pcDNA plasmid |
| | insert of Rz4 (as) |
| SEQ ID NO: 51 | Sequence of oligonucleotide for pcDNA plasmid |
| | insert of Rz5 (as) |
| SEQ ID NO: 52 | Sequence of oligonucleotide for pcDNA plasmid |
| | insert of Rz4 (s) |
| SEQ ID NO: 53 | Sequence of oligonucleotide for pcDNA plasmid |
| | insert of Rz5 (s) |
| SEQ ID NO: 54 | Mouse heparanase |
| SEQ ID NO: 55 | DNA (sense strand) of anti-heparanase ribozyme |
| | (HpaRz2) |
| SEQ ID NO: 56 | Hpa RNA substrate of Rz2 |
| SEQ ID NO: 57 | Human heparanase |

Cells

The methylcholanthrene-induced non-metastatic Eb (L5178Y) T-lymphoma cells (clone 737) were provided by Dr. V. Schirrmacher (DKFZ, Heidelberg, Germany) [Vlodavsky, I. et al. (1983) Cancer Res. 43:2704-11; Larizza, L. et al. (1984) J. Exp. Med. 160:1579-84]. The cells were grown in RPMI 1640 supplemented with 10% FCS, L-glutamine and antibiotics.

Human breast carcinoma MDA-MB-435 and mouse B16-BL6 melanoma cells were purchased from the American Type Culture Association (ATCC, Washington, D.C., USA). Cells were cultured in DMEM (4.5 g glucose per liter) supplemented with 10% FCS, L-glutamine and antibiotics.

Human vascular endothelial EA.Hy926 cells [Edgell (1983) id ibid.; Bouis (2001) id ibid.] were kindly provided by Dr. A. Brill (Dept. of Hematology, Hadassah University Hospital, Jerusalem, Israel) and maintained in DMEM supplemented with 10% FCS and antibiotics at 37° C. and 8.5% CO_{2.} IFN-γ and TNF-α were obtained from Sigma (St. Louis, Mo.) and dissolved in water. Prior to treatment with cytokines, cells were maintained for 8 h in serum-free medium. IFN-γ or TNF-α was added for additional 16 h. Control cultures were treated with vehicle alone.

293T and 3T3 cells were purchased from ATCC.

Animals

Female BALB/c mice were purchased from Harlan Laboratories (Jerusalem, Israel). Hpa-tg mice [Zcharia (2004) id ibid.] were bred at the animal facility of the Hadassah-Hebrew University Medical Center. C57BL/6 mice were purchased from Harlan Laboratories (Jerusalem, Israel). Nude mice were obtained from Harlan Laboratories (Jerusalem, Israel). All animal experiments were approved by the IACUC of the Hadassah-Hebrew University Medical Center.

Synthesis of Anti-hpa Hammerhead Ribozymes

Six anti-hpa hammerhead ribozymes (HpaRz1-6, denoted as SEQ ID NO:19 to 24) were generated by in vitro transcription of single-stranded oligonucleotide template (denoted as SEQ ID NO:34-39; Table 2) encoding the T7 RNA polymerase promoter followed by ribozyme coding sequence. Two μg of each template was transcribed by 10 units of T7 RNA polymerase in a buffer containing 40 mM Tris HCl pH 7.5, 10 mM MgCl₂, 5 mM DTT, 400 μM of dNTPs and 50 μg/ml BSA. Transcription reaction was performed at 37° C. for 30 minutes and stopped by 10 minutes of heat inactivation at 75° C. Reaction products were electrophoresed in denaturing 15% polyacrylamide-gel, visualized by UV-illumination on TLC screen, excised from the gel and purified by ethanol precipitation [Hubinger, G. et al., Exp. Hematol. 29:1226-35 (2001)].

TABLE 2

Sequences of templates for in vitro
transcription

| | | |
|---|---|---|
| SEQ ID NO: | Ribozyme | Sequence |

| | | |
|---|---|---|
| SEQ ID NO:34 | R1 | AGCACCTTTCGGCCTTTCGGCCTCATCAG |
| | | CTCAAGACCTATAGTGAGTCGTATTAC |
| SEQ ID NO:35 | R2 | ACCTACTTTCGGCCTTTCGGCCTCATCAG |
| | | AAGAAGCCTATAGTGAGTCGTATTAC |
| SEQ ID NO:36 | R3 | CTGCTCTTTCGGCCTTTCGGCCTCATCAG |
| | | CCAAGGGCCTATAGTGAGTCGTATTAC |
| SEQ ID NO:37 | R4 | CAAAACTTTCGGCCTTTCGGCCTCATCAG |
| | | TATGGTCCTATAGTGAGTCGTATTAC |
| SEQ ID NO:38 | R5 | AAACTCTTTCGGCCTTTCGGCCTCATCAG |
| | | TGGTCCTCCTATAGTGAGTCGTATTAC |
| SEQ ID NO:39 | R6 | GCTGGATTTCGGCCTTTCGGCCTCATCAG |
| | | AATTGGGCCTATAGTGAGTCGTATTAC |

Synthesis of Labeled hpa-RNA Substrate for Ribozyme Cleavage

To produce the template for hpa-RNA substrate transcription, a 1477 bp (also denoted by SEQ ID NO: 40) fragment was amplified from full length heparanase cDNA and subcloned into pcDNA3 plasmid (Invitrogen, Carlsbad, Calif.) by PCR. Two primers were used: an upper primer containing a T7 RNA polymerase promoter sequence (5′-GTAATACGACTCACTATAGGTGAGCCCCTCGTTCCTGTCCGTCACCAT-3′, also denoted by SEQ ID NO:1) and a lower primer (5′-TTTTATTTTCAGATGCAGCAGC-3′, also denoted by SEQ ID NO:2). PCR conditions were as follows: initial denaturation at 94° C. for 2 min, denaturation at 94° C. for 15 s, annealing for 45 sec at 55° C., and extension for 1 min at 72° C. (30 cycles). Aliquots (15 μL) of the amplified cDNA were separated by electophoresis in 1.5% agarose gel and visualized by ethidium bromide staining [Vlodavsky (1999) id ibid.]. The PCR product of expected size (1477 bp) was isolated and used as a template for in vitro transcription of hpa-RNA substrate for ribozyme cleavage. Transcription was performed with 10 units of T7 RNA polymerase in a buffer containing 40 mM Tris HCl pH 7.5, 10 mM MgCl₂, 5 mM DTT, 400 μM of [³²P] labeled dNTPs and 50 μg/mL BSA at 37° C. for 30 minutes. The reaction was stopped by 10 minutes of heat inactivation at 75° C. The substrate was visualized by electrophoresis in denaturing 5% polyacrylamide gel and subsequent autoradiography.

In Vitro Ribozyine Cleavage Reaction

Analysis of ribozyme cleavage in a cell-free system was performed as described elsewhere [Hubinger (2001) et al.]. Briefly, radioactively labeled hpa-RNA substrate, prepared as described above, was incubated with anti-hpa ribozymes (HpaRz1-6) in a molar ratio of 1:50, at 37° C., for 15 and 60 min. Cleavage products were separated by electrophoresis in denaturing 5% polyacrylamide gel and visualized by autoradiography.

Construction of Ribozyme Expressing Vector

The vectors for expression of anti-hpa ribozyme (HpaRz2) and control ribozyme (ContRz) were constructed by subcloning DNA fragments that encode for HpaRz2 or ContRz into expression vector pcDNA3 with hygromycine B resistance (Invitrogen, Carlsbad, Calif.). The oligonucleotide 5′-AGCTTGGCTTCTTCTGATGAGGCCGAAAGGCCGAAAGTAGGTGC-3′, also denoted by SEQ ID NO:3 and the complementary oligonucleotide 5′-GGCCGCACCTACTTTCGGCCTTTCGGCCTCATCAGAAGAAGCCA-3′, also denoted by SEQ ID NO:4 were used to generate HpaRz2 ribozyme, and oligonucleotides 5′-AGCTTGCGAAGAACTGATGAGGCCGAAAGGCCGAAACAT CCAG-3′, also denoted by SEQ ID NO:5 and 5′-GATCCTGGATGTTTCGGCCTTTCGGCCTCATCAGTTCTTCGCA-3′, also denoted by SEQ ID NO:6 were used to generate the ContRz ribozyme. Each oligonucleotide was annealed to its complement by mixing equal molar amounts, heating to 80° C., and slowly cooling to 30° C. The double-stranded DNA was then sub-cloned into the multicloning site (at HindIII and NotI) of pcDNA3. The sequence of the insert and the region flanking the insert was confirmed by DNA sequencing. Sequences of the oligonucleotides used as inserts into the pcDNA3 plasmid are detailed in Table 3.

TABLE 3

Sequences of oligonucleotides for pcDNA3
plasmid insert

| | | |
|---|---|---|
| SEQ ID NO: | Ribozyme | Sequence |

| | | |
|---|---|---|
| SEQ ID NO:4 | HH-AS-Rz2 | GGCCGCACCTACTTTCGGCCTTTCGG |
| | | CCTCATCAGAAGAAGCCA |
| SEQ ID NO:50 | HH-AS-Rz4 | GGCCGCCAAAACTTTCGGCCTTTCGG |
| | | CCTCATCAGTATGGTCCA |
| SEQ ID NO:51 | HH-AS-Rz5 | GGCCGCAAACTCTTTCGGCCTTTCGG |
| | | CCTCATCAGTGGTCCTCA |
| SEQ ID NO:3 | HH-S-Rz2 | AGCTTGGCTTCTTCTGATGAGGCCGA |
| | | AAGGCCGAAATAGGTGC |
| SEQ ID NO:52 | HH-S-Rz4 | AGCTTGGACCATACTGATGAGGCCGA |
| | | AAGGCCGAAAGTTTTGGC |
| SEQ ID NO:53 | HH-S-Rz5 | AGCTTGAGGACCACTGATGAGGCCGA |
| | | AAGGCCGAAAGAGTTTGC |
| SEQ ID NO:5 | HH-S-CONTR | AGCTTGCGAAGAACTGATGAGGCCGA |
| | | AAGGCCGAAACATCCAGC |
| SEQ ID NO:6 | HH-AS-CONTR | GGCCGCTGGATGTTTCGGCCTTTCGG |
| | | CCTCATCAGTTCTTCGCA |

Construction of siRNA Expression Vectors

The present inventors employed the pSUPER vector (kindly provided by Dr. R. Agami, Division of Tumor Biology, The Netherlands Cancer Institute, Amsterdam, Netherlands) in which siRNA expression is driven by the H1 RNA promoter able to produce small and size-defined RNA transcripts lacking poly-A tails (FIG. 15A). pSUPER is based on pBluecript®KS [Brummelkamp, T. R. et al. (2002) Science 296:550-3]. BglII and HindIII sites were used for cloning of inserts. Upon ligation, BglII site is destroyed.

Mouse siRNA: Oligonucleotides 5′-GATCCCCTCTCAAGTCAACCATGATA TTCAAGAGATATCATGGTTGACTTGAGATTTTTGGAAA-3′, also denoted by SEQ ID NO:7 and 5′-AGCTTTTCCAAAAATCTCAAGTCAACCATGATATCT CTTGAATATCATGGTTGACTTGAGAGGG-3′, also denoted by SEQ ID NO:8 were used to generate mouse anti-hpa siRNA Si1, and oligonucleotides 5′-GATCCCCACTCCAGGTGGAATGGCCCTTCAAGAGAGGGCCATTCCACCTGGA GTTTTTTGGAAA-3′, also denoted by SEQ ID NO:9 and 5′-AGCTTTTCCAAAAAACTCCAGGTGGAATGGCCCTCTCTTGAAGGGCCATTCCA CCTGGAGTGGG-3′, also denoted by SEQ ID NO:10 were used to generate anti-hpa siRNA Si2. Each oligonucleotide pair (100 pmol) was annealed by incubation at 95° C. for 5 min and slow cooling. One μL of this mixture was then ligated into pSUPER vector digested with BglII and HindIII.

pLL 3.7, in which siRNA expression is driven by the U6 RNA promoter able to produce small and size-defined RNA transcripts lacking poly-A tails, was employed for generating pSi-lenti. The oligonucleotides 5′-TACTCCAGGTGGAATGGCCCTTCAAGAGAGGGCCATTCCACCTGGAGTTTTTT C-3′ as denoted by SEQ ID NO:41 and 5′-TCGAGAAAAACTCCAGGTGG AATGGCCCTCTCTTGAAGGGCCATTCCACCTGGAGTA-3′ as denoted by SEQ ID NO:42, were used to generate anti-hpa siRNA pSi2-lenti. Each oligonucleotide pair (100 pmol) was annealed by incubation at 95° C. for 5 minutes and slow cooling. One ll of this mixture was then ligated into pLL 3.7 vector digested with HpaI and XhoI (FIG. 15A).

Human siRNA: oligonucleotides 5′-GATCCCCCCCTGATGTATTGGACATTTTCA AGAGAAATGTCCAATACATCAGGGTTTTTGGAAA-3′, also denoted by SEQ ID NO:30 and 5′-AGCTTTTCCAAAAACCCTGATGTATTGGACATTTCTCTTGAAA ATGTCCAATACATCAGGGGGG-3′, also denoted by SEQ ID NO:31 were used to generate human anti-hpa siRNA H1, and oligonucleotides 5′-GATCCCCACTTCTAAGAAAGTCCACCTTCAAGAGAGGTGGTCTTTCTTAGAAG TTTTTTGGAAA-3′, also denoted by SEQ ID NO:32 and 5′-AGCTTTTCCA AAAAACTTCTAAGAAAGTCCACCTCTCTTGAAGGTGGTCTTTCTTAGAAGTGG G-3′, also denoted by SEQ ID NO:33 were used to generate anti-hpa siRNA H2. Each oligonucleotide pair (100 pmol) was annealed by incubation at 95° C. for 5 min and slows cooling. One μl of this mixture was then ligated into pSUPER vector.

Plasmid Constructs

The 1.9-kb human heparanase promoter region [Hpse (-1791/+109)-LUC] was subcloned upstream of the luciferase (LUC) gene in a pGL2 basic reporter plasmid (Promega, Madison, Wis.), as described [Elkin (2003) id ibid.]. The plasmid containing the LUC gene driven by a CMV enhancer/promoter (CMV-LUC) was kindly provided by Dr. A. Oppenheim (Hadassah-Hebrew University Medical Center, Jerusalem, Israel). Anti-heparanase siRNA expression vector pSi2 and the control pSUPER vector were generated as described.

Generation and Titer of Lentiuirus

Lentiviral production was performed as described [Lois, C. et al. (2002) Science 295:868-872]. Briefly, pLL3.7 and packaging vectors were co-transfected into 293T cells and the resulting condition medium collected 36 hours later. Virus was recovered by ultracentrifugation for 1.5 h at 25,000 rpm in a Beckman SW28 rotor and resuspended in PBS (15-200 μl). Titers were determined by infecting 3T3 cells with serial dilutions of concentrated lentivirus. GFP expression of infected cells was determined by flow cytometry 48 hours after infection. For a typical preparation, the titer was approximately 4-10×10⁸ infectious units (IFU) per ml.

Matrigel Invasion Assay

Tumor cells were assayed for Matrigel invasion at 37° C. in a 5% CO₂ incubator for 6 h, using blind-well chemotaxis chambers and polycarbonate filters (13 mm in diameter, 8 μg/m pore size) (Costar) coated with Matrigel as described [Elkin, M. et al. (1999) Clin. Cancer Res. 5:1982-8; Albini, A. et al. (1987) Cancer Res. 47:3239-45]. Medium conditioned by 3T3 fibroblasts was applied as a chemo-attractant and placed in the lower compartment of the Boyden chamber [Elkin (1999) id ibid.]. Cells on the lower surface of the filter were stained and counted by examination of five microscopic fields. When Eb lymphoma cells were tested, these were first incubated (48 h, 37° C.) with [³H]-thymidine (1 μCi/ml) (Arnersham) and cell invasion was quantified by counting the Matrigel coated filters in a β scintillation counter [Goldshmidt, O. et al. (2003) id ibid].

Heparanase Activity

For measurements of heparanase enzymatic activity, tissue extract or cell lysates were incubated in dishes coated with sulfate-labeled ECM, prepared as described [Vlodavsky (1983) id ibid.]. Briefly, bovine corneal endothelial cells were established and cultured at 37° C. in a 10% CO₂ humidified incubator in DMEM (1 g of glucose/liter) supplemented with 10% calf serum (Life Technologies, Grand Island, N.Y.) and 5% dextran T-40 in the presence of Na₂[³⁵S]O₄ (25 μCi/ml) (Amersham Pharmacia Biotech, Buckinghamshire, UK), added on days 1 and 5 after seeding. The sub-endothelial ECM was exposed by dissolving the cell layer with PBS containing 0.5% Triton X-100 and 20 mM NH4OH, followed by four washes in PBS. Equal protein aliquots of cell lysates prepared from 1×10⁶ cells by three cycles of freezing and thawing in heparanase reaction buffer (20 mM phosphate-citrate buffer containing 1 mM dithiothreitol, 1 mM CaCl₂, and 50 mM NaCl) were incubated (3 h, 37° C., pH 6.6) with the resulting ³⁵S-labeled ECM. Sulfate-labeled material released into the incubation medium was analyzed by gel filtration on a Sepharose 6B column [Vlodavsky (1983) id ibid.]. Nearly intact heparan sulfate proteoglycans are eluted just after the void volume (peak I, Kav<0.2, fractions 1-10). Heparan sulfate degradation fragments produced by heparanase are eluted later with 0.5<Kav<0.8 (peak II, fractions 15-35) [Vlodavsky (1983) id ibid.]. Reaction buffer with or without recombinant human heparanase (1 ng/ml) was routinely used as a positive or negative control, respectively.

For the analysis of heparanase activity in tumor cells, tumor cell lysates were incubated (5 h, 37° C., pH 6.6) with ³⁵S-labeled ECM, prepared as described [Vlodavsky (1999a) id ibid]. The incubation medium was centrifuged and the supernatant containing sulfate-labeled HS degradation fragments analyzed by gel filtration on a Sepharose CL-6B column (0.9×30 cm). Fractions (0.2 ml) were eluted with PBS and their radioactivity counted in a β-scintillation counter [Vlodavsky (1999a) id ibid.; Vlodavsky (1994) id ibid.; Vlodavsky (1983) id ibid.].

For all the heparanase activity measurements, each experiment was performed at least three times and the variation in elution positions (Kₐᵥ values) did not exceed ±15%.

Cell Adhesion

Eb cells were grown (1×10⁶ cells/ml, 48 h, 37° C.) in RPMI medium supplemented with 10% FCS in the presence of [³H] thymidine (1 μCi/ml) (Amersham). Eb labeled cells were washed (×3) free of unincorporated thymidine and incubated (37° C., pH 7.2) in complete medium for 15 min in ECM coated wells [Vlodavsky, I. (1999b) Current Protocols in Cell Biology. Vol. 1, John Wiley & Sons, New York, pp. 10.4.1-4.4]. After incubation, the wells were washed (×3) with serum-free medium and the remaining firmly attached cells were solubilized (2 h, 0.2 M NaOH, 37° C.) and counted in a β-scintillation counter.

Experimental Metastasis

Six week-old male C57BL/6 mice were injected into the lateral tail vein with 0.4 ml of cell suspension containing 0.4×10⁶ B16-BL6 melanoma cells transiently transfected with anti-hpa siRNA expressing vector (pSi2) or empty pSUPER vector [Brummelkamp (2002) id ibid.] Five mice were used per group. Fifteen days after cell injection, mice were sacrificed, their lungs removed, fixed in Bouin's solution, and scored for the number of metastatic nodules on the lung surface under a dissecting microscope [Vlodavsky (1994) id ibid.].

Spontaneous Metastasis

Two month-old male CD1 nude mice were inoculated subcutaneously into the lower back with 1×10⁶ Eb-lymphoma cells expressing secreted chimeric-hpa protein (cHpaEb), stably transfected with either anti-hpa ribozyme (pHpaRz2) or control ribozyme (pContRz) expression plasmids (10 mice per group). Five mice of each group were monitored for survival rate. The additional 5 mice were sacrificed on day 11 and examined for primary tumor size, vascularity and liver metastasis [Goldshmidt, O. et al. (2002) id ibid.]. Metastatic colonization of the liver was evaluated by gross examination, weight measurements and microscopic inspection of tissue sections.

All animal experiments were approved by the Animal Care Committee of the Hebrew University (Jerusalem, Israel).

RNA Isolation from Tumor Cells and RT-PCR

RNA was isolated from tumor cells with TRIzol (Life Technologies) according to the manufacturer's instructions and quantitated by ultraviolet absorption. After oligo(dT)-primed reverse transcription of 500 ng total RNA, the resulting single stranded cDNA was amplified using TaqDNA polymerase and buffer (Promega). The primers used were: HPU-355: 5′-TTCGATCCCAAGAAGGAATCAAC-3′, also denoted by SEQ ID NO:11 and HPL-229: 5′-GTAGTGATGCCATGTAACTGAATC-3′, also denoted by SEQ ID NO:12 for human heparanase; 431-U: 5′-ATGCTCTAC AGTTTTGCCAAGTG-3′, also denoted by SEQ ID NO:13 and 876-L:5′-CAGAATTTTTTGCACAGAGAGAA-3′, also denoted by SEQ ID NO:14 for mouse heparanase; GAPDH-S: 5′-CCACCCATGGCAAAATTCCATGGCA-3′, also denoted by SEQ ID NO:15 and GAPDH-AS: 5′-TCTAGACGGCAGGTCAGGTCCACC-3′, also denoted by SEQ ID NO:16 for human GAPDH and L-19-U: 5′-ATGCCAACTCTCGTCAACAG-3′, also denoted by SEQ ID NO:17 and L-19-L: 5′-GCGCTTTCGTGCTTCCTT-3′, also denoted by SEQ ID NO:18 for mouse L19 cDNA. The PCR conditions for human cDNA were an initial denaturation of 4 min at 94° C. and subsequent denaturation for 45 sec at 94° C., annealing for 1 min at 60° C. and extension for 1 min at 72° C. (26 cycles). PCR conditions for mouse cDNA were an initial denaturation of 2 min at 95° C. and subsequent denaturation for 15 sec at 96° C., annealing for 70 sec at 58° C. and extension for 80 sec at 72° C. (24 cycles). Aliquots (10 μL) of the amplification products were separated by 1.5% agarose gel electrophoresis and visualized by ethidium bromide staining. Only RNA samples that gave completely negative results in PCR without reverse transcriptase were further analyzed.

RNA Isolation from EA.Hy926 Cells and Semi-Quantitative RT-PCR Analysis

As described above, RNA was isolated with TRIzol (Life Technologies) according to the manufacturer's instructions and was quantified by ultraviolet absorption. Oligo (dT)-primed reverse transcription was performed using 1 μg total RNA in a final volume of 20 μl and the resulting cDNA was further diluted to 100 μl. Comparative semi-quantitative PCR was performed as follows: L19 cDNA was first amplified at low cycle number human L19 primer sequences: L-19-U (5′-ATGCCAACTCTCGTCAACAG-3′; SEQ ID NO:17) and L-19-L (5′-GCGCTTTCGTGCTTCCTT-3′; SEQ ID NO:18)]. The resulting PCR products were visualized by electrophoresis and ethidium bromide staining, and the intensity of each band was quantified using Scion Image software (Scion, Frederick, Md.). If needed, cDNA dilutions were adjusted and L19 RT-PCR products were re-amplified in order to obtain similar intensities for L19 signals with all the samples. The adjusted amounts of cDNA were used for PCR with primers HPU-355 (TTCGATCCCAAGAAGGAATCAAC; SEQ ID NO:11) and HPL-229 (GTAGTGATGCCATGTAACTGAATC; SEQ ID NO:12), designed to amplify a 564-bp PCR product specific for human heparanase [Vlodavsky (1999a) id ibid.]. Aliquots of 10 μl of the amplification products were separated by 1.5% agarose gel electrophoresis and visualized by ethidium bromide staining. Only RNA samples that gave completely negative results in PCR without reverse transcriptase were further analyzed, to rule out the presence of genomic DNA contamination. The intensity of each band was quantified using Scion Image software. Results are expressed as band intensity relative to that of L19. The PCR conditions were: initial denaturation of 4 minutes at 94° C., followed by 26 cycles of denaturation for 45 seconds at 94° C., annealing for 1 minute at 60° C., and extension for 1 minute at 72° C.

Immunostaining

MDA-435 cells transfected with pHpaRz2 or pContRz were seeded on round glass coverslips in 4-well plates. Twenty four hours later the cells were washed twice with PBS and fixed with chilled (−20° C.) 100% methanol for 3 min. Following fixation, cells were washed (×5) with PBS and intrinsic fluorescence was blocked with 50 mM NH₄Cl for 5 min. Cells were then washed (×3) with PBS, incubated (30 min, 24° C.) with 5% goat serum, and washed twice with PBS. Slides were incubated (2 h, 24° C.) with polyclonal anti-human heparanase antibodies (Ab 733, 10 μg/ml) [Zetser, A. et al. (2004) J. Cell Sci. 117:2249-2258], washed (×5) with PBS and incubated with Cy-3-conjugated goat anti-rabbit IgG (1:100, Jackson, Bar-Harbor, Me.) for 1 h at 24° C. Slides were then washed 8 times with PBS, mounted with 90% glycerol in PBS, and visualized with a Zeiss LSM 410 confocal microscope.

Transfection

Eb lymphoma (0.5×10⁶ cells/ml) or MDA-435 breast carcinoma (0.3×10⁶ cells/ml) were incubated (48-72 h, 37° C.) with a total of 1-2 μg DNA and 6 μL Fugene transfection reagent (Boehringer, Mannheim, Germany) in 94 μl Optimem (Gibco-BRL, Invitrogen) [Albini (1987) id ibid.]. Transfected Eb cells were selected with 200 μg/ml hygromycine B (Sigma) and 350 μg/ml G418 (Gibco-BRL). Transfected MDA-435 cells were selected with 200 μg/ml hygromycine B (Sigma). Stable transfected cells were obtained and routinely maintained in selection medium, to avoid overgrowth of nontransfected cells. B16-BL6 melanoma cells were electroporated with pSi1, pSi2 or empty pSUPER constructs (4×10⁶ cells in 400 μL medium containing 10 μg of plasmid DNA) by a single 70 msec pulse at 140 V, using ECM 830 electro square porator and disposable cuvettes (model 640; 4-mm gap) (BTX, Inc.). Following electroporation, the transfected cells were plated at a density of 0.4×10⁶ cells/10 cm and allowed to grow for 2-48 h. Efficiency of transfection (˜80%) was evaluated by electroporation of a GFP expressing vector.

Delayed-Type Hypersensitivity (DTH) Assay

DTH reactions were induced in the ear skin of 5-6 week-old female BALB/c mice or in hpa-transgenic (hpa-tg) mice and their wild type counterparts. Five week-old female mice were sensitized on the shaved abdominal skin with 100 μl of 2% oxazalone dissolved in acetone/olive oil [4:1 (vol/vol)] applied topically [Lider (1990) id ibid.]. DTH assay was performed 5 days later by challenging the mice with 20 μl of 0.5% oxazalone in acetone/olive oil, 10 μl administered topically to each side of the ear. Thickness of a constant area of the ear was measured with Mitutoyo engineer's micrometer immediately before challenging, 24 hours after challenge and then every other day for 5 days. The increase in ear thickness over baseline levels (thickness of the ears treated with vehicle alone) was used as a parameter for the extent of inflammation.

Intradermal Injection and In Vivo Electroporation

Mice were anesthetized using isoflurane inhalation. Plasmid DNA (25 μl per site, at concentration 1 μg/μl) was intradermally injected with a 28-gauge needle into the dorsal skin of the mice. The in vivo electroporation system (Genetronics) consisted of a square wave pulse generator (ECM 830) and a caliper electrode (P/N 384) was applied topically in four different directions. Electric pulses (100V; 20 ms) were charged four times at intervals of one second.

For in vivo electroporation in the ear, mice were anesthetized and plasmid DNA was intradermally injected with a 0.3 ml syringe and 30 gauge needle into the mouse ear (20 μg per site in 25 μl of PBS). To keep variability to a minimum, the same skilled operator performed all injections. A 30 second time interval lapsed between injection and initiation of electroporation. The in vivo electroporation system (Genetronics Inc., San Diego, Calif.) consisted of a square wave pulse generator (ECM 830) and a caliper electrode, applied topically. The caliper electrode (modes 384; BTX/Harvard Apparatus, Holliston, Mass.) consists of two 1 cm² brass plate electrodes. The electroporation was performed by squeezing the ear between two plates and applying six pulses of 75 V with a pulse length of 20 msec and interval of 1 second, and polarity reversal after three pulses.

Permeability Assay

DTH challenged (n=5) and untreated (n=8) mice were injected intravenously with 100 μl Evans blue dye (30 mg/kg in 100 μl PBS (Sigma)) at 24 h or 7 days after oxazolone challenge. Thirty minutes later, mice were anesthetized with a mixture of ketamine (800 μg/10 g body weight Ketaset; Fort Dodge Laboratories, Fort Dodge, Iowa) and avertin (0.5 μg/10 g body weight 2,2,2,-tribromoethanol in 2.5% t-amyl alcohol) (Sigma). The intensity of vascular permeability was analyzed macroscopically.

Histology

Ear tissue was collected immediately after the mice were sacrificed, fixed in 4% buffered formaldehyde, embedded in paraffin, and sectioned (5 μm sections). After deparaffinization and rehydration, sections were washed (3×) with PBS and stained with hematoxylin/eosin or Masson-Trichrom, as described [Zcharia (2004) id ibid.].

Immunohistochemistry

Immunohistochemical staining was performed as described [Vlodavsky, (1999a) id ibid.; Zcharia (2005) id ibid.], with minor modifications. Briefly, 5 μm ear tissue sections, prepared as described above, were incubated in 3% H₂O₂, denatured by boiling (3 min) in a microwave oven in citrate buffer (0.01 M, pH 6.0), and blocked with 10% goat serum in PBS. Sections were incubated with polyclonal anti-heparanase antibody (Ab #733, diluted 1:100 in 10% goat serum in PBS), raised against a synthetic peptide ¹⁵⁸KKFKNSTYRSSSVD¹⁷¹ (SEQ ID NO:43) [Zetser (2004) id ibid.] located at the N-terminus of the 50 kDa subunit of the heparanase enzyme). Color was developed through the Zymed AEC substrate kit (Zymed Laboratories, South San Francisco, Calif.) for 10 min, followed by counter staining with Mayer's hematoxylin.

Luciferase Assay

Mice ears were removed just before or 48 h after the DTH challenge with oxazolone. The ears were snap frozen in liquid nitrogen and pulverized to a fine powder with a liquid nitrogen-cooled pestle. The powder was resuspended in 100 μl of ice-cold Reporter Lysis Buffer (Promega Corp., Madison, Wis.), frozen and thawed 3 times, and centrifuged for 20 min at 4° C. at 14,000 rpm. Supernatant was transferred to a new tube, lysate protein content was determined and 25 μl samples were assayed for LUC activity using the Luciferase Reporter Assay system (Promega). LUC activity was calculated as light units/unit protein, which yields values similar to those based on internal beta-galactosidase transfection standards [Nawaz, Z. et al. (1999) Cancer Res. 59:372-376]. Data are presented as the means of at least three determinations, and all experiments were repeated at least twice with similar results.

Induction of Hair Cycle

Depilation was used to induce hair growth in resting follicles, as described [Paus, R. et al. (1990) Br. J. Dermatol. 122:777-784]. Dorsal skin of 8-week-old female C57BL/6 mice at the telogen phase (as identified by their pink skin color) was depilated using Hair Remover Wax Strip Kit (Del Laboratories, Farmingdale, N.Y.), leading to the synchronized development of anagen hair follicles.

Statistic Analysis

Statistical evaluations employed un-paired Student's t test. All P values were two-sided.

### Example 1

Selection of Active Anti-hpa Hammerhead Ribozymes

In order to investigate the effect of silencing heparanase expression in different aspects involved in heparanase activity, the inventors produced six different hammerhead ribozymes (HpaRz1-6, Table 4). The ribozymes were synthesized by in vitro transcription, using double-stranded DNA oligonucleotides containing the T7 promoter, the conserved catalytic domain, and two flanking sequences designed to recognize specific motifs along the human heparanase mRNA, as shown for HpaRz2 (FIG. 1A). The targeting sequences for generating the ribozymes were as follows: HpaRz1, from nucleotides 586-600 of the human heparanase sequence, HpaRz2, from nucleotides 589-603, HpaRz3, from nucleotides 729-743, HpaRz4, from nucleotides 881-895, HpaRz5, from nucleotides 883-897, and HpaRz6, from nucleotides 1194-1208 (Table 5). All nucleotide positions refer to the human heparanase sequence as denoted by GenBank Accession No. AF144325.1 (SEQ ID NO:57). To associate with and cleave its specific target, the hammerhead ribozyme must fold into a typical three-dimensional structure, with the folded catalytic core domain connected to the flanking complementary sequences, as shown for HpaRz2 in FIG. 1B.

TABLE 4

Sequence name and sequence of Ribozymes
HpaRz1-6

| | | |
|---|---|---|
| Name | Rybozyme | Sequence 5′-3′ |

| | | |
|---|---|---|
| SEQ ID NO:19 | HpaRz2 | GCUUCUUCUGAUGAGGCCGAAAGGCCGAA |
| | | AGUAGGU |
| SEQ ID NO:20 | HpaRz1 | UCUUGAGCUGAUGAGGCCGAAAGGCCGAA |
| | | AGUGCT |
| SEQ ID NO:21 | HpaRz3 | CCCUUGGCUGAUGAGGCCGAAAGGCCGAA |
| | | AGAGCAG |
| SEQ ID NO:22 | HpaRz4 | GACCAUACUGAUGAGGCCGAAAGGCCGAA |
| | | AGUUUUG |
| SEQ ID NO:23 | HpaRz5 | AGGACCACUGAUGAGGCCGAAAGGCCGAA |
| | | AGAGUUU |
| SEQ ID NO:24 | HpaRz6 | CCCAAUUCUGAUGAGGCCGAAAGGCCGAA |
| | | ATCCAGC |

To test the effectiveness of ribozyme cleavage, the inventors generated a truncated heparanase RNA substrate of 1477 nt (SEQ ID NO:40) containing recognition sites for all six anti-hpa ribozymes (not shown). While the substrate showed no specific cleavage when incubated without ribozyme, distinct cleavage fragments were easily detectable following incubations with all six ribozymes created (HpaRz1-6). As shown by FIG. 1C, the most effective cleavage was performed by HpaRz2, which was, therefore, selected for further studies.

TABLE 5

Target sequences on human heparanase

| | | |
|---|---|---|
| SEQ ID NO: | Nucleotides | Sequence (5′-3′) |

| | | |
|---|---|---|
| SEQ ID NO:44 | 586-600 | AGCACCTACTCAAGA |
| SEQ ID NO:45 | 589-603 | ACCTACTCAAGAAGC |
| SEQ ID NO:46 | 729-743 | CTGCTCTTCCAAGGG |
| SEQ ID NO:47 | 881-895 | CAAAACTCTATGGTC |
| SEQ ID NO:48 | 883-897 | AAACTCTATGGTCCT |
| SEQ ID NO:49 | 1194-1208 | GCTGGATAAATTGGG |

### Example 2

Stable Expression of Anti-Heparanase Ribozyme Inhibits Heparanase Activity and Cell Invasion

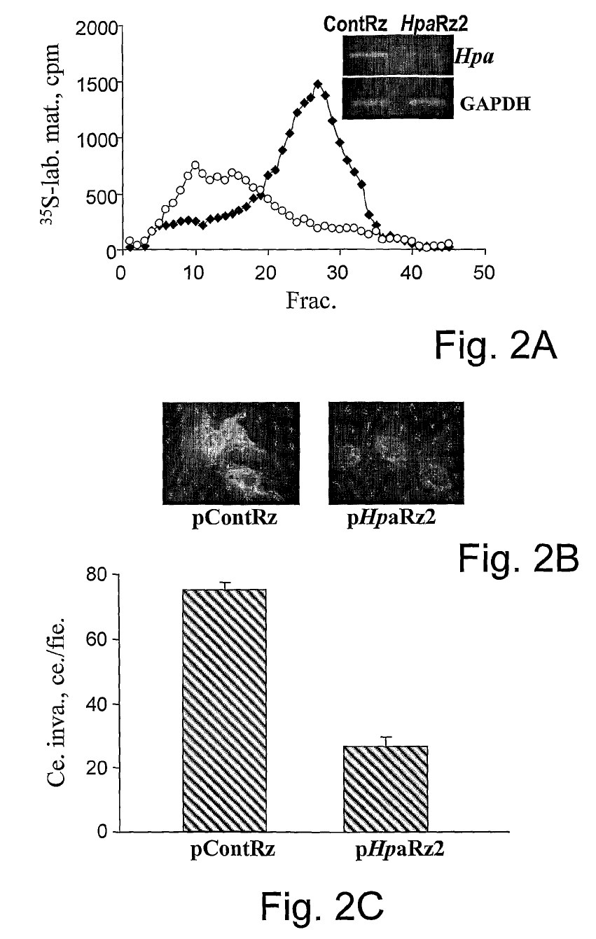

The inventors constructed a vector for constitutive expression of anti-heparanase ribozyme HpaRz2 (pHpaRz2) and tested its ability to inhibit endogenous heparanase synthesis. For this purpose, the inventors stably transfected MDA-435 breast carcinoma cells, known to express high levels of heparanase [Vlodavsky (1999a) id ibid.], with the pHpaRz2 vector and assayed the transfected cells for heparanase mRNA expression and enzymatic activity (FIG. 2A). As a control for possible effects of the hammerhead itself, the MDA-435 cells were transfected with pContRz vector, which encodes a ribozyme with identical catalytic core, but incapable of recognizing the heparanase mRNA (FIG. 2A). Stable expression of HpaRz2 in MDA-435 cells led to marked (˜80%) decrease in heparanase mRNA levels, evaluated by RT-PCR (FIG. 2A, inset) and densitometric analysis (not shown), and completely abolished heparanase enzymatic activity (FIG. 2A) as compared to cells transfected with a ContRz plasmid (FIG. 2A). Immunofluorescent staining with anti-heparanase antibodies revealed a marked decrease in heparanase protein content in pHpaRz2 transfected MDA-435 cells, as compared to cells transfected with pContRz (FIG. 2B).

Since heparanase plays a role in cell invasion through the ECM and BM [Vlodavsky (1999a) id ibid.; Goldshmidt (2002) id ibid.], the inventors next tested the effect of anti-hpa ribozyme on MDA-435 invasive capacity. Cells transfected with pHpaRz2 or pContRz were compared for their ability to invade a reconstituted BM (Matrigel) [Albini (1987) id ibid.]. As demonstrated in FIG. 2C, stable expression of HpaRz2 led to a 64% decrease in the number of cells that invaded the Matrigel (HpaRz2-transfected cells: 26.8 cells per field; 95% CI=24.2 to 29.4 vs. control cells expressing ContRz: 75.2 cells per field; 95% CI=72.8 to 77.6). The decrease was highly significant (P<0.0001). Similar results were obtained with C-6 rat glioma cells, engineered to express high levels of human heparanase (data not shown).

### Example 3

Anti-Heparanase Ribozyme Decreases Lymphoma Primary Tumor Vascularization, Metastasis and Mortality of the Tumor Bearing Mice

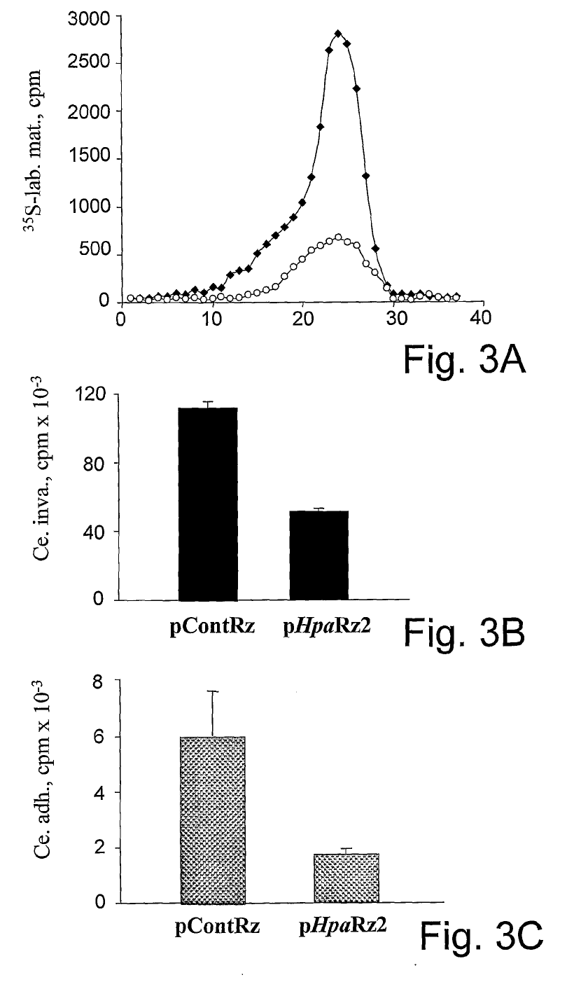

The inventors next tested the effectiveness of the anti-heparanase ribozyme approach in vivo and in particular its anti-metastatic potential. Eb lymphoma cells were used, which lack endogenous heparanase, and were engineered to express a readily secreted chimeric form of heparanase (cHpa), composed of the human enzyme and the chicken heparanase signal sequence [Goldshmidt, O. et al. (2001) J. Biol. Chem. 276:29178-87]. The inventors have recently demonstrated that cHpa expressing Eb cells (cHpaEb) degraded HS in the ECM to a much higher extent than non-transfected cells, or cells transfected with the human enzyme [Goldshmidt (2001) id ibid.]. Moreover, cHpaEb cells exhibited increased invasiveness and metastatic potential in mice [Goldshmidt (2002) id ibid.] and are, therefore, regarded as a useful experimental model for the study of heparanase in tumor progression. In the present invention, cHpaEb cells stably transfected with pHpaRz2 or pContRz were tested for heparanase activity (FIG. 3A) and ability to invade Matrigel (FIG. 3B). Stable expression of HpaRz2 in these cells led to a marked (>70%) decrease in heparanase-mediated degradation of HS, demonstrating the efficient targeting of the secreted form of heparanase. As demonstrated in FIG. 3B, stable transfection of cHpaEb cells with HpaRz2 led to a pronounced, highly significant decrease (P<0.0001) in the number of cells that invaded the Matrigel (50.9×10³ cpm; 95% CI=49.1×10³ to 52.7×10³, as compared to cells expressing ContRz: 112.2×10³ cpm; 95% CI=108.6×10³ to 115.8×10³).

Recently, heparanase was demonstrated to promote cell adhesion to ECM, independently of its enzymatic properties [Goldshmidt (2003) id ibid.]. To test the effect of HpaRz2 on heparanase-mediated cell adhesion, the inventors compared the adhesive ability of cHpaEb cells stably transfected with pHpaRz2 vs. pContRz constructs. As shown in FIG. 3C, HpaRz2 effectively inhibited adhesion of the transfected cells to dishes coated with naturally produced ECM.

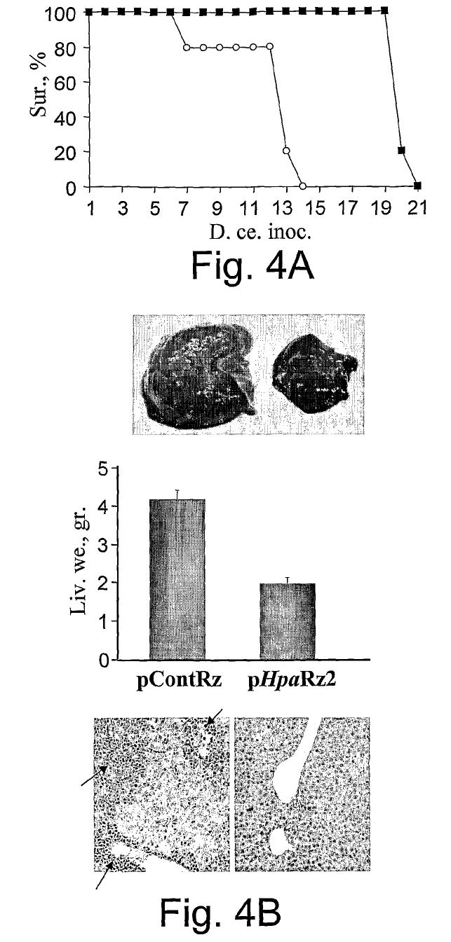

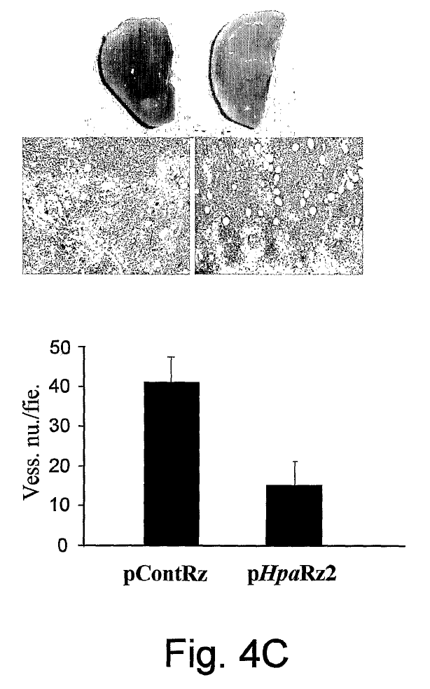

The inventors next investigated the effect of ribozyme-mediated heparanase gene silencing on the metastatic potential of cHpaEb lymphoma cells. For this purpose, cells transfected with either pHpaRz2 or pContRz were inoculated subcutaneously into nude mice. The mice were tested for survival time and liver metastasis. As shown in FIG. 4A, all mice injected with pHpaRz2 transfected cells survived during the first 3 weeks of the experiment. In a striking contrast, 100% mortality was observed in mice inoculated with cells transfected with control, inactive ribozyme, already on day 14 of the experiment (FIG. 4A).

On day 11, livers of five additional mice from each group were removed, weighed and processed for histological examination (FIG. 4B). Gross macroscopic examination of the liver revealed numerous lymphoma metastasis in 100% of mice inoculated with pContRz transfected cells vs. few or no visible metastatic nodules in the liver of mice injected with pHpaRz2-transfected cells (FIG. 4B, top). HpaRz2-mediated decrease in the metastatic ability of cHpaEb lymphoma was also reflected by a significant (P<0.0001) difference in liver weight between mice injected with pHpaRz2-, vs. pContRz-transfected cells (1.98 gr; 95% CI=1.86 to 2.1 gr vs. 4.66 gr; 95% CI=4.47 to 5.85 gr; FIG. 4B, middle). Microscopic examination of liver tissue sections confirmed a massive infiltration of the liver by cells transfected with pContRz vs. little or no liver infiltration by cells transfected with pHpaRz2 (FIG. 4B, bottom).

In addition, the mice were examined for vascularity of the primary tumor. A marked decrease in blood content and hemorrhage was noted in tumors produced by cHpaEb cells transfected with pHpaRz2, as compared to tumors produced by cells transfected with pContRz (FIG. 4C). Whereas tumors produced by ContRz-expressing cells were dark-reddish, tumors generated by HpaRz2-expressing cells appeared pale (FIG. 4C, Top). The decreased vascularity of tumors produced by active vs. control ribozyme-transfected cells was confirmed by histological examination of the respective tissue sections stained with anti-Von Willebrand Factor antibody (FIG. 4C, middle). Vessel counting revealed a significant (P<0.0001) difference in vascular density in tumors produced by pHpaRz2-transfected cHpaEb cells (39.3; 95% CI=32.9 to 45.7) vs. tumors produced by pContRz-transfected cells (15; 95% CI=11.2 to 18.8; FIG. 4C, bottom).

### Example 4

Effect of siRNA-Mediated Heparanase Silencing on Invasive and Metastatic Potential of B16-BL6 Melanoma Cells

To further elucidate the direct involvement of heparanase in tumor progression and the effectiveness of endogenous heparanase gene silencing approach, the inventors applied the siRNA targeting approach, utilizing B16-BL6 mouse melanoma cells, characterized by high levels of endogenous heparanase [Vlodavsky (1994) id ibid.; Miao (1999) id ibid.]. Two siRNA variants (Si1 and Si2), targeting two different regions of the mouse heparanase mRNA, were designed and cloned into the pSUPER plasmid [Brummelkamp (2002) id ibid.] to generate pSi1- and pSi2-expression vectors.

Table 6 summarizes the siRNAs described herein. The specified sequences represent the corresponding DNA templates.

TABLE 6

| | | |
|---|---|---|
| Name | siRNA | Sequence 5′-3′ |

| | | |
|---|---|---|
| SEQ ID NO:26 | Mouse siRNA | GATCCCCTCTCAAGTCAAGTCAACCA |
| | si1 (5′) | TGATATTCAAGAGATATCATGGTTGA |
| | | CTTGAGATTTTTGGAAA |
| SEQ ID NO:27 | Mouse siRNA | TTCCAAAAAT CTCAAGTCAA |
| | si1 (3′) | CCATGATATCT CTTGAATATCATGG |
| | | TTGACTTGAGAGGG |
| SEQ ID NO:28 | Mouse siRNA | GATCCCCACTCCAGGTGGAATGGCCC |
| | si2 (5′) | TTCAAGAGAGGGCCATTCCACCTGGA |
| | | GTTTTTTGGAAA |
| SEQ ID NO:29 | Mouse siRNA | TTCCAAAAAACTCCAGGTGGAATGGC |
| | si2 (3′) | CCTCTCTTGAAGGGCCATTCCACCTG |
| | | GAGTGGG |
| SEQ ID NO:30 | Human siRNA | GATCCCCCCCTGATGTATTGGACATT |
| | H1 (5′) | TTCAAGAGAAATGTCCAATACATCAG |
| | | GGTTTTTGGAAA |
| SEQ ID NO:31 | Human siRNA | AGCTTTTCCAAAAACCCTGATGTATT |
| | H1 (3′) | GGACATTTCTCTTGAAAATGTCCAAT |
| | | ACATCAGGGGGG |
| SEQ ID NO:32 | Human siRNA | GATCCCCACTTCTAAGAAAGTCCACC |
| | H2 (5′) | TTCAAGAGAGGTGGTCTTTCTTAGAA |
| | | GTTTTTTGGAAA |
| SEQ ID NO:33 | Human siRNA | AGCTTTTCCAAAAAACTTCTAAGAAA |
| | H2 (3′) | GTCCACCTCTCTTGAAGGTGGTCTTT |
| | | CTTAGAAGTGGG |

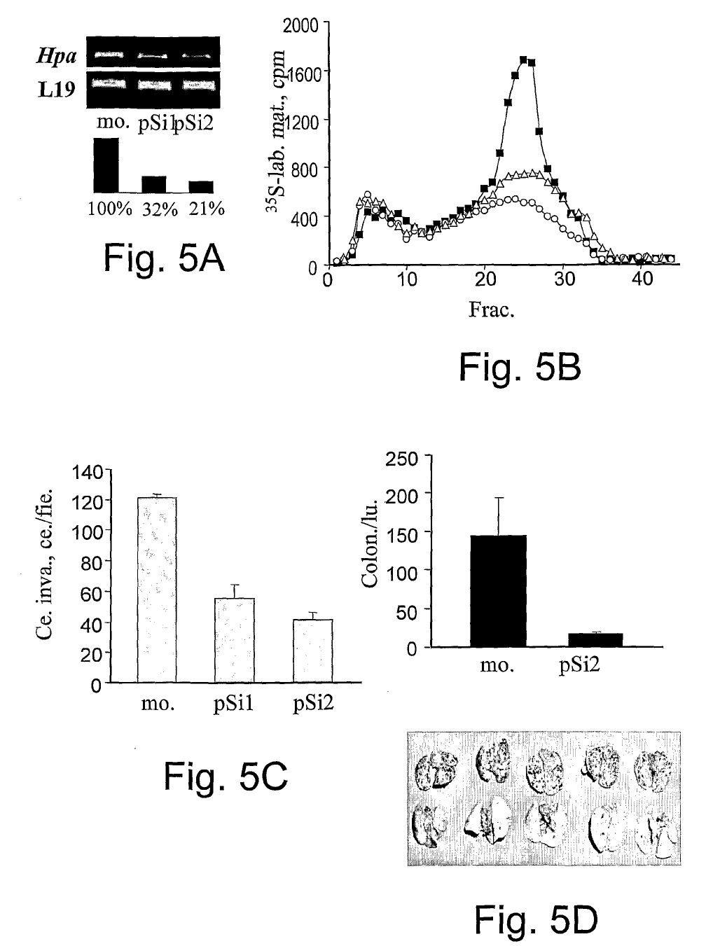

B16-BL6 mouse melanoma cells were transiently transfected with pSi1, pSi2, or empty pSUPER (mock) by electroporation, and 48 h later the cells were tested for heparanase expression and activity. Semi-quantitative RT-PCR revealed a 70-80% decrease in heparanase mRNA levels in cells transfected with pSi1 or pSi2, as compared to mock-transfected cells (FIG. 5A). Heparanase enzymatic activity measured in lysates of pSi1- and pSi2-transfected cells was markedly lower (˜60%) than in mock-transfected cells, further demonstrating effective silencing of the heparanase gene by siRNA (FIG. 5B).

In subsequent experiments, the inventors tested the effect of hpa targeted siRNA on B16-BL6 cell invasiveness using the Matrigel invasion assay. As demonstrated in FIG. 5C, the ability of B16-BL6 cells to invade through Matrigel-coated filters was significantly inhibited (P<0.0011) following transfection with pSi1 (57.4 cells per field; 95% CI=48.2 to 66.6), or pSi2 (40.6 cells per field; 95% CI=35.6 to 45.6), as compared to cells transfected with vector alone (121.4 cells per field; 95% CI=118.9 to 123.9). Finally, the effect of siRNA-mediated heparanase silencing on experimental metastasis in vivo was tested by the inventors. For this purpose, B16-13L6 cells were transfected through electroporation with either the pSi2 plasmid, or with the pSUPER vector alone and 48 h later the cells were injected into the tail vein of C57BL/6 mice (0.4×10⁶ cells per mouse in 0.4 ml PBS). Eleven days later, the mice were sacrificed and their lungs evaluated for the number of surface metastatic colonies. As demonstrated in FIG. 5D, expression of hpa targeted siRNA (Si2) effectively (˜90%) and significantly (P<0.0001) inhibited lung colonization of B16-BL6 melanoma cells (16 colonies; 95% CI=13.4 to 18.6 in mice injected with pSi² transfected cells vs. 144.3 colonies; 95% CI=95.1 to 193.5 in mice injected with mock transfected cells). Collectively, these data clearly demonstrate that specific silencing of endogenous heparanase gene expression effectively inhibits the invasive and metastatic potential of B16-BL6 cells.

### Example 5

Effect of In Vivo siRNA-Mediated Heparanase Silencing on Hair Growth and Inflammatory Response

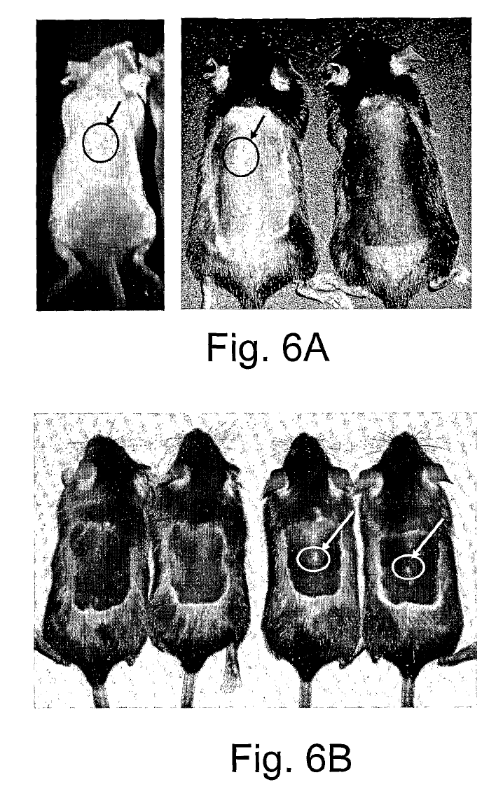

As shown recently by the present inventors [WO2004/006949], heparanase significantly induces hair growth. Therefore, the effect of in vivo inhibition of heparanase induced hair growth was next examined using two different vectors encoding the siRNA of the invention and two different procedures for applying the siRNA molecules on mice skin. The dorsal skin of 8-week-old female C57BL/6 mice at the telogen phase (as identified by their pink skin color) was depilated using Hair Remover in order to induce hair growth. As shown by FIG. 6A, anti-heparanase pSi2 construct in the pSUPER plasmid that was injected into skin and transfected using an in vivo electroporation system (FIG. 6A, middle), clearly inhibits hair growth compared to control GFP and empty plasmids (FIG. 6A, right and left) that were introduced using the same procedure. Inhibition of hair growth by the siRNA of the invention was further demonstrated using the pSi2-Lenti viral vector injected intradermally. As shown in FIG. 6B (right), inhibition of heparanase expression by the siRNA of the invention inhibits hair growth, particularly compared to PBS control (FIG. 6B, left).

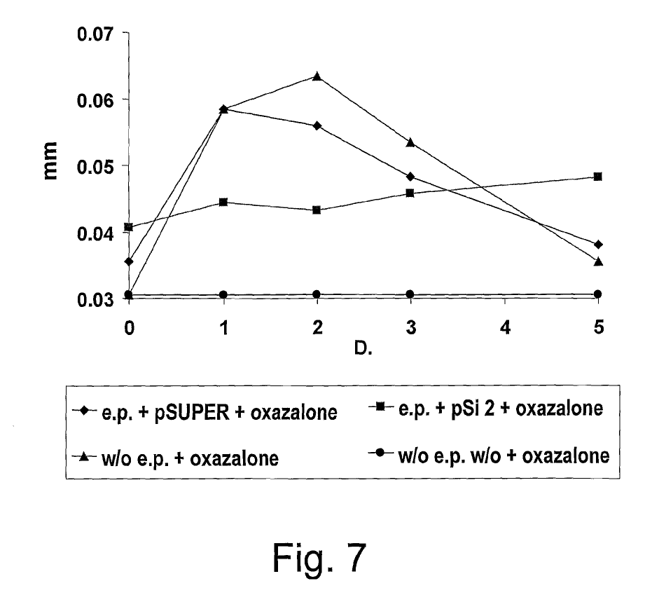

Preliminary experiments were then performed to study heparanase involvement in DTH in vivo. Female BALB/c mice were sensitized by application of oxazalone on the shaved abdominal skin. Five days later, mice were challenged by oxazalone and electroporated with empty vector (pSUPER) or pSi2. Thickness of a constant area of the ear was measured immediately before challenge, 24 hours after challenge and then every other day for 5 days. As shown in FIG. 7, application of oxazalone in the presence of the Si2 plasmid clearly reduced the ear thickness, indicating that the siRNA of the invention inhibits heparanase expression in vivo and results in the inhibition of an inflammatory response.

### Example 6

Association between DTH Reactivity and Heparanase Levels

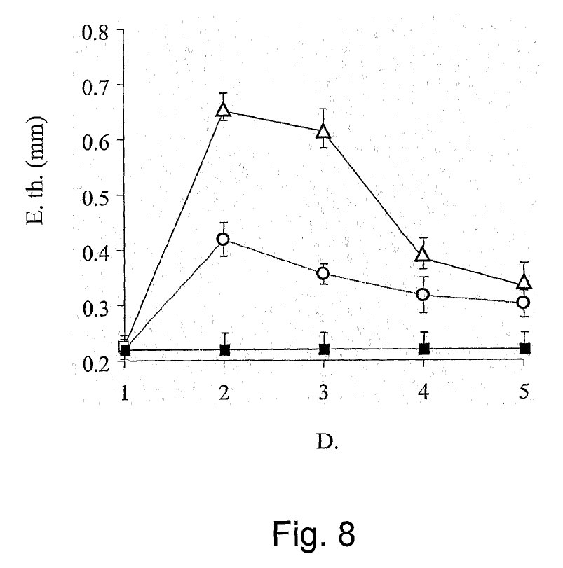

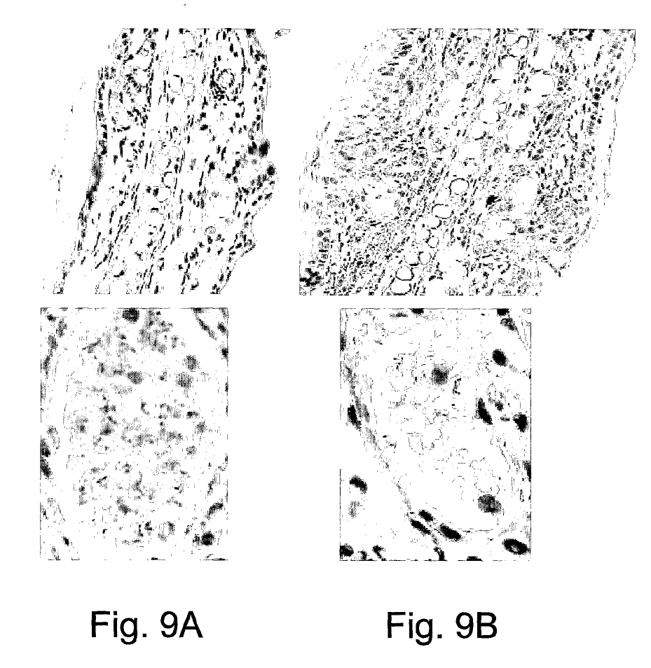

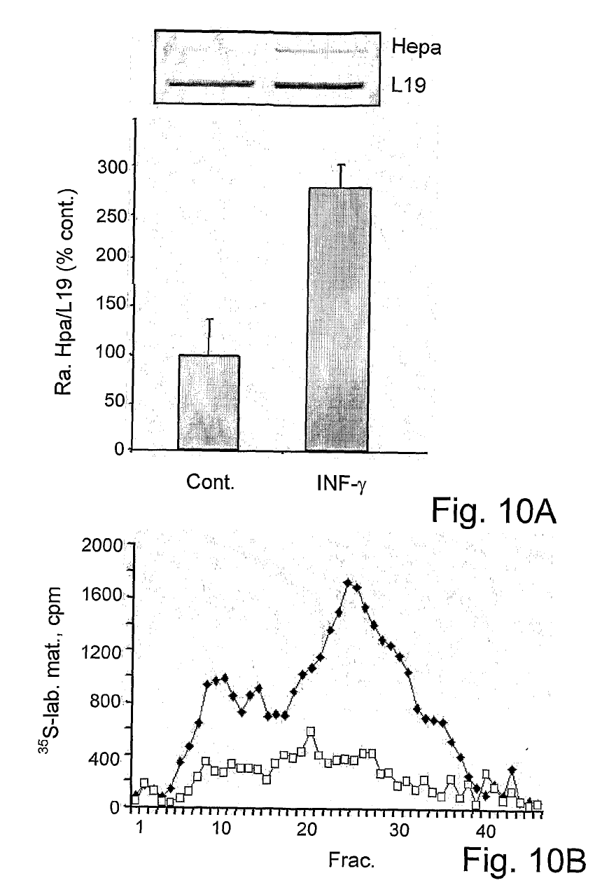

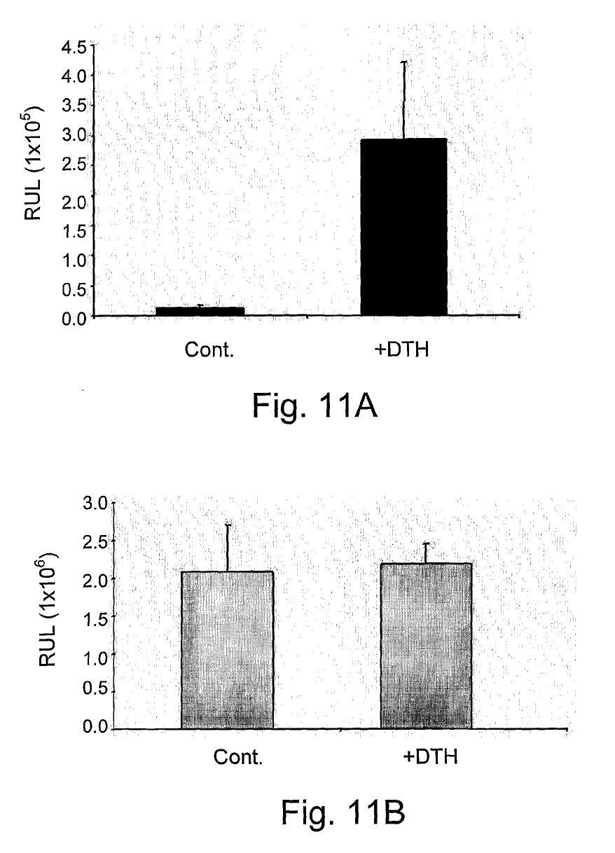

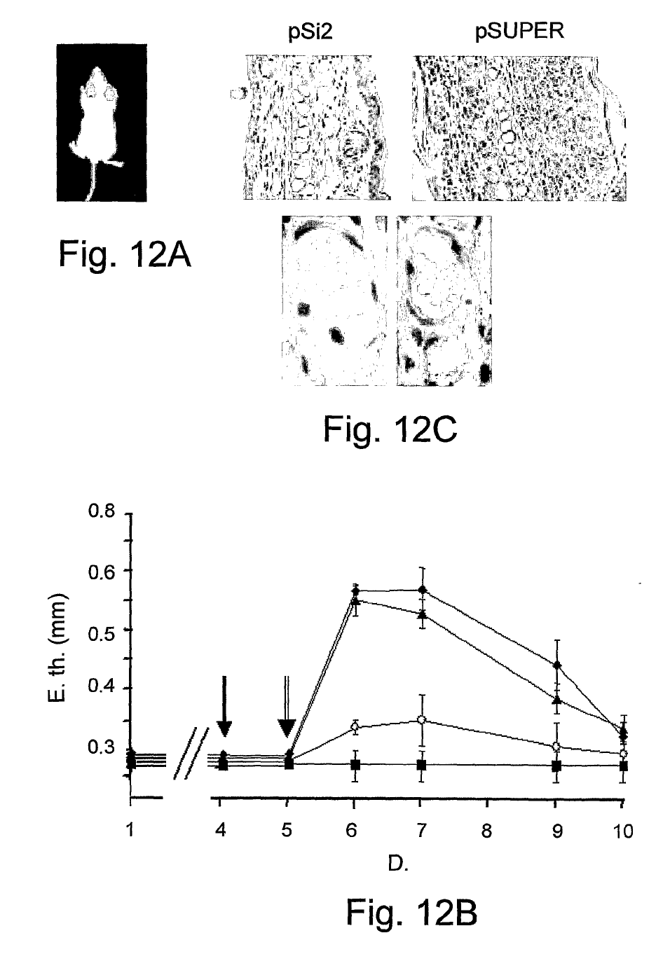

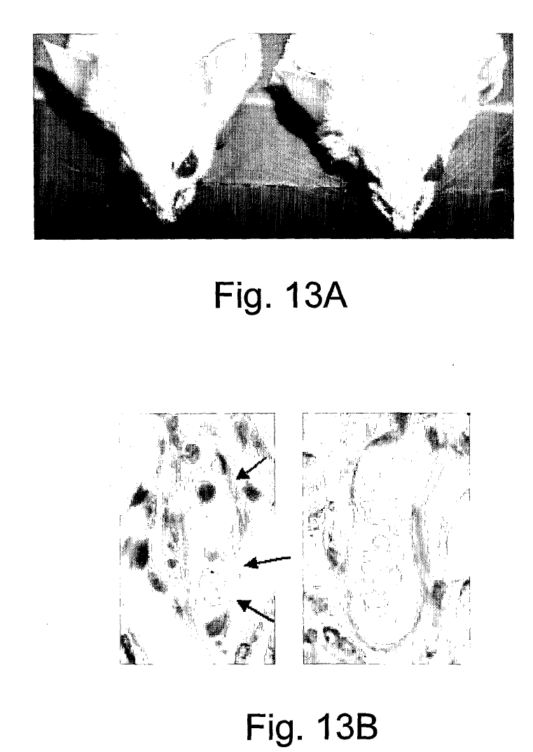

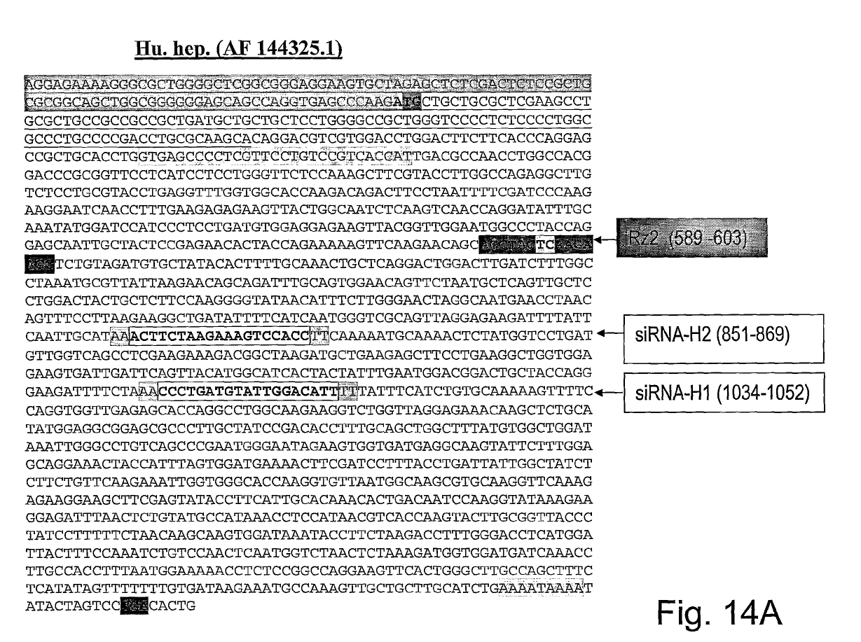

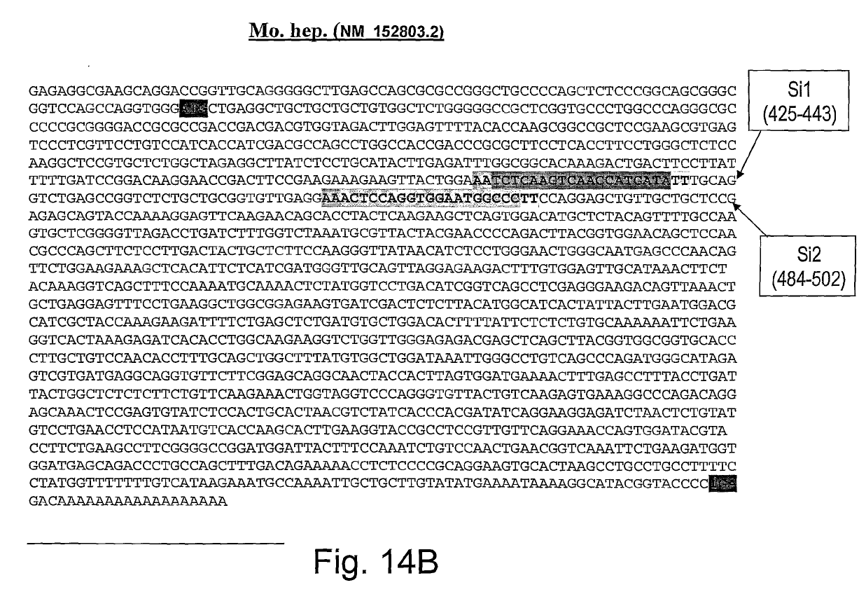

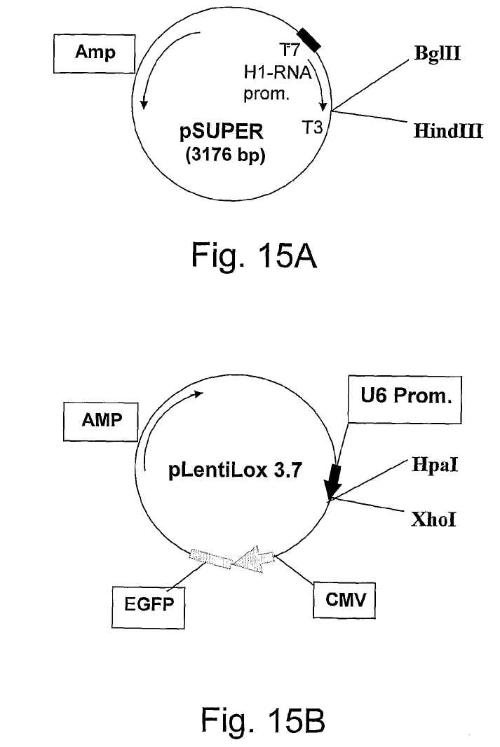

DTH reactivity was first studied in a recently generated homozygous transgenic (hpa-tg) mice overexpressing human heparanase in all tissues [Zcharia (2004) id. ibid.]. Hpa-tg mice and their wild-type counterparts were sensitized with the hapten oxazolone, as described in Experimental Procedures, and the DTH reaction was elicited 5 days later by applying oxazolone onto the ears. Twenty-four hours after the oxazolone challenge, a markedly enhanced inflammatory response and edema formation were detected in hpa-tg mice in comparison with wild-type mice, as reflected by a 3.5 fold increase in ear thickness in the hpa-tg mice vs. a 2-fold increase in wild type mice (FIG. 8). The differences in the extent of edema formation between the two groups of mice remained statistically significant for 3 days after challenge (FIG. 8). These results prompted the determination of the levels of endogenous heparanase during DTH induction in wild type mouse ears. As shown in FIG. 9A-9B, high levels of the heparanase protein were detected by immunostaining (anti-heparanase Ab 733) [Zetser (2004) id. ibid.] in the ears in which inflammation has been elicited by oxazolone, as compared to low levels or absence of heparanase in control, unchallenged ears (FIG. 9A, 9B). Notably, a greater part of tissue elements expressing elevated levels of heparanase in the dermis of DTH-affected ears was represented by capillary vascular endothelium (FIG. 9B, bottom). Sebaceous glands were stained in all sections, due to a non-specific absorption of the anti-heparanase antibody, as found by others with different antibodies [Philp, D. et al. (2004) id ibid.].

### Example 7

IFN-γ Induces Heparanase Expression in Endothelial Cells In Vitro

Since interferon γ (IFN-γ) is regarded as a key mediator of the DTH reaction [Black (1999) id ibid.; Fong, T. A., and T. R. Mosmann (1989) J. Immunol. 143:2887-2893], the inventors next investigated the effect of IFN-γ on heparanase expression in endothelial cells in vitro. For this purpose, one of the best characterized vascular endothelial cell lines, the EA.hy926 cells were used [Edgell, C. J. et al. (1983) Proc. Natl. Acad. Sci. USA 80:3734-3737; Bouis, D. et al. (2001) Angiogenesis 4:91-102] was used. EA.hy926 cells were treated (or not) with IFN-γ for 24 hours and then tested for heparanase mRNA expression. Semi-quantitative RT-PCR reaction revealed that IFN-γ treatment yielded a 3-fold increase in heparanase mRNA content, as compared to untreated cells (FIG. 10A). Treatment with tumor necrosis factor a (TNF-α), another major inducer of DTH reactivity [Black (1999) id ibid.], yielded a 2-fold increase in heparanase expression by EA.hy926 cells (not shown), in agreement with the previously reported ability of TNF-α to augment heparanase expression in other types of endothelial cells [Chen, G. D. et al. (2004) Biochemistry 43:4971-4977]. Next the levels of heparanase enzymatic activity were determined in EA.hy926 endothelial cells, untreated or treated with IFN-γ, to verify the RT-PCR observations. Heparanase activity was tested by incubation (3 h, 37° C.) of cell lysate samples with a metabolically sulfate-labeled ECM. Sulfate-labeled degradation products released into the incubation medium were subjected to gel filtration on Sepharose 6B columns [Vlodavsky (1999a) id ibid.; Vlodavsky (1983) id ibid.]. The substrate alone consisted almost entirely of nearly intact, high-molecular-weight material eluted just after the void volume (peak I, fractions 1-10, Kav<0.2). This material (peak I) was previously shown to be the result of proteolytic activity residing in the ECM itself and/or expressed by the cells [Vlodavsky (1992) id ibid.]. The elution pattern of labeled material released during incubation of lysed untreated cells with sulfate-labeled ECM, showed little or no heparanase enzymatic activity (FIG. 10B). In contrast, high heparanase activity was detected in lysates of IFN-γ treated cells, as indicated by a 3-fold increase in release from ECM of low-molecular-weight sulfate-labeled fragments (peak II, fractions 20-35, 0.5<Kav<0.8; FIG. 10B) [Vlodavsky (1999a) id ibid.; Vlodavsky (1983) id ibid.]. These fragments were shown to be degradation products of heparan sulfate, as they were 5-6 fold smaller than intact heparan sulfate side chains, resistant to further digestion with papain and chondroitinase ABC, and susceptible to deamination by nitrous acid [Vlodavsky (1983) id ibid.]

### Example 8

Heparanase Promoter Activation During DTH Inflammation

In order to test whether heparanase induction during inflammation occurs due to a transcriptional activation of the heparanase gene, the in vivo electroporation technique was applied. This technique is based on injection of the expression vector into the ear, which is followed by application of an electric field also into the ear [Zcharia (2005) id ibid.; Zhang L. et al. (2002) Biochim. Biophys. Acta. 1572: 1-9], in order to deliver the LUC reporter gene driven by the heparanase promoter (Hpse-LUC) [Elkin (2003) id ibid], prior to DTH elicitation. Four days following sensitization with oxazolone, the ears of Balb/C mice in the experimental group were electroporated with the Hpse-LUC construct. Ears of mice in the control group were electroporated with construct containing the LUC gene under a constitutive CMV promoter (CMV-LUC) [Zcharia (2005) id ibid.]. Twenty-four hours later, left ears of the mice in both experimental and control groups were challenged with oxazolone, while the right ears were left untreated. Fourty-eight hours after challenge, when a strong DTH-associated swelling was readily detected in all ears challenged with oxazolone, but not in non-challenged ears (not shown), the mice were sacrificed and the ears removed and lysed. Lysates were normalized for total protein content and luciferase activity was measured as described in Experimental Procedures. As shown in FIG. 11A, DTH induction in left ears provoked a marked activation of the heparanase promoter, yielding a 23-fold increase (P<0.003) in LUC activity measured in left vs. right ears of mice from the experimental group. In contrast, in the ears of mice from control group electroporated with a CMV-LUC construct, DTH induction did not result in any statistically significant change in LUC activity (FIG. 4B), indicating that the difference observed in the experimental group was heparanase promoter-specific and not due to variation in transfection efficiency. These data indicate that the increase in heparanase levels in DTH inflammation occurs through activation of the heparanase gene promoter.

### Example 9

Local Silencing of Heparanase Profoundly Decreases Inflammatory Response In Vivo

To explore the effect of local heparanase silencing on DTH reactivity, the anti-hpa siRNA expressing vector pSi2 was delivered to Balb/C mouse ears, 24 hours prior to challenge with the hapten. pSi2 was delivered through electroporation, as described in Experimental Procedures. To demonstrate that this technique ensures the actual delivery of electroporated DNA and its uniform expression in the ear tissue, ears of male Balb/C mice were electroporated first with a CMV-LUC construct, encoding the luciferase gene under the constitutive CMV promoter, and the expression of luciferase in mouse ears visualized in vivo using a CCCD camera (FIG. 12A), as described [Zcharia (2005) id ibid.].

In the subsequent set of experiments, 6 week old male Balb/C mice were sensitized with oxazolone and divided into three groups (n=5 mice per group), 4 days post sensitization. The first and second groups were electroporated with anti-heparanase siRNA expression vector (pSi2) and with empty vector (pSUPER) [Brummelkamp (2002) id ibid.], respectively; mice in the third group were not subjected to electroporation. Twenty four hours later, ears in all three groups were challenged with the hapten. Hapten was also applied onto the ears of additional five mice, which have not been previously sensitized or electroporated, serving as a negative control group. The ear thickness was monitored for five consecutive days (FIG. 12A). Twenty-four hours post challenge, a marked inflammatory response was detected in both the pSUPER-electroporated and non-electroporated ears, reflected by more than a two-fold increase in ear thickness, as compared with the control group. In contrast, in the ears electroporated with the anti-heparanase siRNA encoding vector pSi2, the inflammatory response was significantly inhibited, as reflected by a 96% decrease in ear swelling and edema formation, compared to ears electroporated with the pSUPER vector (FIG. 12B). A statistically significant difference in the extent of ear swelling between pSi2- and pSUPER-electroporated ears persisted throughout the 5 consecutive days of the experiment.

To follow the changes in local heparanase expression levels and to ensure that electroporation of pSi2 resulted in heparanase gene silencing throughout the in vivo experiment, heparanase immunostaining of tissue sections of the ears in which DTH was induced following electroporation with pSi2 or pSUPER vectors was compared. As expected, intense heparanase staining was observed in pSUPER-electroporated ears (FIG. 12C right), vs. a very weak or no heparanase staining in pSi2-electroporated ears (FIG. 12C left), similar to that observed in normal untreated ears (FIG. 9A). These results demonstrate that siRNA mediated heparanase silencing inhibits DTH reactivity in vivo.

### Example 10

Heparanase Silencing Inhibits Vessel Permeability During DTH

Since reduced inflammatory response (reflected by a very limited ear swelling) was found, following heparanase silencing in wild type mice, as well as increased edema formation in hpa-tg mice, the inventors investigated whether heparanase directly affects vascular leakage, a hallmark of the early phase of inflammation. The ears of oxazolone-sensitized Balb/C mice were electroporated with pSi2-(left ear) or pSUPER-(right ear) vectors on day 4 post sensitization. Twenty four hours later, both the right and left ears were challenged with oxazolone, and after additional 16 hours mice were injected intravenously with Evans blue. As shown in FIG. 13A, 16.5 hours after DTH elicitation by oxazolone challenge, vascular leakage was significantly higher in pSUPER- than in pSi2-electroporated ears, as reflected by a marked difference in Evans blue extravasation. Macroscopically, a strong DTH-associated swelling was readily detected in all pSUPER-, but not in pSi2-electroporated ears (not shown). Partial disruption of the sub-endothelial basement membrane surrounding capillary vessels was clearly noted by Masson-Trichrom staining and histological examination in the pSUPER-electroporated ears (FIG. 13B left, arrows). In contrast, in pSi2-electroporated ears the subendothelial BM remained undamaged. These findings indicate that increased heparanase activity expressed by activated endothelial cells at the site of inflammation disrupts the permeaselective properties of the subendothelial BM and thereby enables vessel leakage during inflammation.

1. A nucleic acid molecule comprising at least one target specific sequence, which sequence is complementary to a target ribonucleotide sequence comprised within heparanase mRNA.

2. The nucleic acid molecule according to claim 1, wherein said nucleic acid molecule is a ribonucleic acid molecule selected from the group consisting of a ribonucleic acid molecule having endonuclease activity and a small interfering KNA (siRNA).

3. The nucleic acid molecule according to claim 2, wherein said ribonucleic acid molecule having endonucleasc activity is a ribozyme, preferably a hammerhead ribozyme which specifically cleaves heparanase RNA and thereby inhibits the expression of heparanase.

4. The nucleic acid molecule according to claim 3, wherein said ribozyme comprises three contiguous regions, a first region, a second region and a third region, where at least a portion of the first and the third regions is complementary to said target RNA sequence within heparanase, and at least a portion of the second region is a ribozyme catalytic domain.

5. The nucleic acid molecule according to claim 4, wherein said ribozyme comprises a ribonucleic acid sequence selected from the group consisting of SEQ ID NO: 19, 20, 21, 22, 23, 24 and any derivatives or functional fragments thereof.

6. The nucleic acid molecule according to claim 5, wherein said ribozyme comprises the ribonucleic acid sequence as denoted by SEQ ID NO: 19 or any analog, variant, derivative and fragment thereof.

7. The nucleic acid molecule according to claim 6, wherein said ribozyme has the ribonucleic acid sequence as denoted by SEQ ID NO: 19 and is designated HpaRz2.

8. The nucleic acid molecule according to claim 2, wherein said ribonucleic acid molecule is siRNA comprising a double strand ribonucleic acid (dsRNA) sequence, wherein at least a portion of one strand of said dsRNA comprises a sequence complementary to a sequence within the heparanase mRNA sequence.

9. The nucleic acid molecule according to claim 8, wherein said siRNA leads to specific cleavage of heparanase RNA and thereby inhibits the expression of heparanase.

10. The nucleic acid molecule according to claim 9, wherein said siRNA comprises a dsRNA sequence selected from the group consisting of a dsRNA composed of one strand comprising the sequence as denoted by SEQ ID NO: 26 and a second complementary strand comprising the sequence as denoted by SEQ ID NO: 27 and a dsRNA composed of one strand comprising the sequence as denoted by SEQ ID NO: 28 and a second complementary strand comprising the sequence as denoted by SEQ ID NO: 29.

11. The nucleic acid molecule according to claim 10, wherein said siRNA is composed of one strand having the sequence as denoted by SEQ ID NO: 26 and a complementary strand having the sequence as denoted by SEQ ID NO: 27, and is designated si1.

12. The nucleic acid molecule according to claim 10, wherein said siRNA is composed of one strand having the sequence as denoted by SEQ ID NO: 28 and a complementary strand having the sequence as denoted by SEQ ID NO: 29, and is designated si2.

13. An expression vector comprising a polynucleotide sequence encoding a nucleic acid molecule as defined in claim 1, which vector optionally further comprises at least one of an operably linked promoter, a transcription start region, a transcription termination region and further regulatory elements.

14. A host cell transformed or transfected with the expression vector of claim 13.

15. A composition for the inhibition of heparanase expression, comprising as an active ingredient one of a nucleic acid molecule as defined in claim 1; an expression vector including a polynucleotide sequence encoding the nucleic acid molecule, which vector optionally further comprises at least one of an operably linked promoter, a transcription start region, a transcription termination region and further regulatory elements; and a host cell transformed or transfected with the expression vector.

16. The composition according to claim 15, optionally further comprising a pharmaceutically acceptable carrier, diluent, excipient and/or additive.

17. The composition according to claim 16, for medical use.

18. The pharmaceutical composition of claim 17, for the treatment or the inhibition of a process or a pathologic disorder associated with heparanase expression.

19. The pharmaceutical composition according to claim 18, wherein said process associated with heparanase expression is one of angiogenesis, tumor formation, tumor progression and tumor metastasis.

20. The pharmaceutical composition according to claim 18, wherein said pathologic disorder associated with heparanase expression is one of a malignant proliferative disorder, an inflammatory disorder, a kidney disorder and an autoimmune disorder.

21. The pharmaceutical composition according to claim 20, wherein said malignant proliferative disorder is any one of solid and non-solid tumor selected from the group consisting of carcinoma, sarcoma, melanoma, leukemia and lymphoma.

22. Use of one of a nucleic acid molecule as defined in claim 1; an expression vector including a polynucleotide sequence encoding the nucleic acid molecule, which vector optionally further comprises at least one of an operably linked promoter, a transcription start region, a transcription termination region and further regulatory elements; and a host cell transformed or transfected with the expression vector, as an agent for the inhibition of heparanase expression.

23. Use of one of a nucleic acid molecule as defined in claim 1;
an expression vector including a polynucleotide sequence encoding the nucleic acid molecule, which vector optionally further comprises at least one of an operably linked promoter, a transcription start region, a transcription termination region and further regulatory elements;
and a host cell transformed or transfected with the expression vector, in the preparation of a composition for the inhibition of heparanase expression.

24. Use of one of a nucleic acid molecule as defined in claim 1;
an expression vector including a polynucleotide sequence encoding the nucleic acid molecule, which vector optionally further comprises at least one of an operably linked promoter, a transcription start region, a transcription termination region and further regulatory elements; and a host cell transformed or transfected with the expression vector, in the preparation of a pharmaceutical composition for the treatment or the inhibition of a process or a pathologic disorder associated with heparanase expression, said composition optionally further comprising a pharmaceutically acceptable carrier, diluent, excipient and/or additive.

25. The use according to claim 24, wherein said process associated with heparanase expression is any one of angiogenesis, tumor formation, tumor progression and tumor metastasis.

26. The use according to claim 24, wherein said pathologic disorder associated with heparanase expression is one of a malignant proliferative disorder, an inflammatory disorder, a kidney disorder and an autoimmune disorder.

27. The use according to claim 26, wherein said malignant proliferative disorder is one of solid and non-solid tumor selected from the group consisting of carcinoma, sarcoma, melanoma, leukemia and lymphoma.

28. A method for the inhibition of heparanase expression comprising the step of in vivo or in vitro contacting a heparanase encoding nucleic acid sequence, under suitable conditions, with an inhibitory effective amount of a nucleic acid molecule as defined in claim 1, or with a composition comprising as an active indient one of the nucleic acid molecule; an expression vector including a polynucleotide sequence encoding the nucleic acid molecule, which vector optionally further comprises at least one of an operably linked promoter, a transcription start region a transcription termination region and further regulatory elements; and a host cell transformed or transfected with the expression vector.

29. A method for the inhibition of heparanase expression in a subject in need thereof comprising the step of administering to said subject an inhibitory effective amount of a nucleic acid molecule as defined in claim 1, or with a composition comprising as an active ingredient one of the nucleic acid molecule; an expression vector including a polynucleotide sequence encodin the nucleic acid molecule, which vector optionallv further comprises at least one of an operably linked promoter, a transcription start region, a transcription termination region and further regulatory elements; and a host cell transformed or transfected with the expression vector.

30. A method for the inhibition or treatment of a process or a pathologic disorder associated with heparanase expression comprising the step of administering to a subject in need thereof a therapeutically effective amount of a nucleic acid molecule as defined in claim 1, or with a composition comprising as an active ingredient one of the nucleic acid molecule; an expression vector including a polynucleotide sequence encoding the nucleic acid molecule, which vector optionally further comprises at least one of an operably linked promoter, a transcription start region, a transcription termination region and further regulatory elements; and a host cell transformed or transfected with the expression vector.

31. The method according to claim 30, wherein said process associated with heparanase expression is any one of angiogenesis, tumor formation, tumor progression and tumor metastasis.

32. The method according to claim 30, wherein said pathologic disorder associated with heparanase expression is one of a malignant proliferative disorder, an inflammatory disorder, an autoimmune disorder and a kidney disorder.

33. The method according to claim 32, wherein said malignant proliferative disorder is any one of solid and non-solid tumor selected from the group consisting of carcinoma, sarcoma, melanoma, leukemia and lymphoma.

34. The method according to claim 32, wherein said inflammatory disorder is delayed-type hypersensitivity.


SEQUENCE LISTING (57 sequences)

SEQ ID NO: 1 - DNA, 48 bp, Artificial Sequence
  5′ Primer for T7 polymerase
gtaatacgactcactataggtgagcccctcgttcctgtccgtcaccat

SEQ ID NO: 2 - DNA, 22 bp, Artificial Sequence
  3′ Primer for T7 polymerase
ttttattttcagatgcagcagc

SEQ ID NO: 3 - DNA, 44 bp, Artificial Sequence
  5′ Primer for preparation of HpaRz2 ribozyme
agcttggcttcttctgatgaggccgaaaggccgaaagtaggtgc

SEQ ID NO: 4 - DNA, 44 bp, Artificial Sequence
  3′ Primer for preparation of HpaRz2 ribozyme
ggccgcacctactttcggcctttcggcctcatcagaagaagcca

SEQ ID NO: 5 - DNA, 43 bp, Artificial Sequence
  5′ Primer for preparation of control ribozyme
agcttgcgaagaactgatgaggccgaaaggccgaaacatccag

SEQ ID NO: 6 - DNA, 43 bp, Artificial Sequence
  3′ Primer for preparation of control ribozyme
gatcctggatgtttcggcctttcggcctcatcagttcttcgca

SEQ ID NO: 7 - DNA, 64 bp, Artificial Sequence
  5′-Primer for preparation of mouse siRNA si1
gatcccctctcaagtcaaccatgatattcaagagatatcatggttgacttgagatttttggaaa

SEQ ID NO: 8 - DNA, 64 bp, Artificial Sequence
  3′ Primer for preparation of mouse siRNA si1
agcttttccaaaaatctcaagtcaaccatgatatctcttgaatatcatggttgacttgagaggg

SEQ ID NO: 9 - DNA, 64 bp, Artificial Sequence
  5′ Primer for preparation of mouse siRNA si2
gatccccactccaggtggaatggcccttcaagagagggccattccacctggagttttttggaaa

SEQ ID NO: 10 - DNA, 64 bp, Artificial Sequence
  3′ Primer for preparation of mouse siRNA si2
agcttttccaaaaaactccaggtggaatggccctctcttgaagggccattccacctggagtggg

SEQ ID NO: 11 - DNA, 23 bp, Artificial Sequence
  HPU-355 primer
ttcgatcccaagaaggaatcaac

SEQ ID NO: 12 - DNA, 24 bp, Artificial Sequence
  HPL-229 primer
gtagtgatgccatgtaactgaatc

SEQ ID NO: 13 - DNA, 23 bp, Artificial Sequence
  431-U primer
atgctctacagttttgccaagtg

SEQ ID NO: 14 - DNA, 23 bp, Artificial Sequence
  876-L primer
cagaattttttgcacagagagaa

SEQ ID NO: 15 - DNA, 25 bp, Artificial Sequence
  GAPDH-S primer
ccacccatggcaaaattccatggca

SEQ ID NO: 16 - DNA, 24 bp, Artificial Sequence
  GAPDH-AS primer
tctagacggcaggtcaggtccacc

SEQ ID NO: 17 - DNA, 20 bp, Artificial Sequence
  L-19-U primer
atgccaactctcgtcaacag

SEQ ID NO: 18 - DNA, 18 bp, Artificial Sequence
  L-19-L primer
gcgctttcgtgcttcctt

SEQ ID NO: 19 - RNA, 36 nt, Artificial Sequence
  HpaRz2
gcuucuucugaugaggccgaaaggccgaaaguaggu

SEQ ID NO: 20 - DNA, 36 bp, Artificial Sequence
  HpaRz1
ucuugagcugaugaggccgaaaggccgaaaggugct

SEQ ID NO: 21 - RNA, 36 nt, Artificial Sequence
  HpaRz3
cccuuggcugaugaggccgaaaggccgaaagagcag

SEQ ID NO: 22 - RNA, 36 nt, Artificial Sequence
  HpaRz4
gaccauacugaugaggccgaaaggccgaaaguuuug

SEQ ID NO: 23 - RNA, 36 nt, Artificial Sequence
  HpaRz5
aggaccacugaugaggccgaaaggccgaaagaguuu

SEQ ID NO: 24 - DNA, 36 bp, Artificial Sequence
  HpaRz6
cccaauucugaugaggccgaaaggccgaaatccagc

SEQ ID NO: 25 - RNA, 37 nt, Artificial Sequence
  Control Ribozyme pContRz
gcaagaacugaugaggccgaaaggccgaaacauccag

SEQ ID NO: 26 - DNA, 64 bp, Artificial Sequence; identical to SEQ ID NO: 7
  Mouse siRNA si1 (5′)

SEQ ID NO: 27 - DNA, 59 bp, Artificial Sequence
  Mouse siRNA si1 (3′)
ttccaaaaatctcaagtcaaccatgatatctcttgaatatcatggttgacttgagaggg

SEQ ID NO: 28 - DNA, 64 bp, Artificial Sequence; identical to SEQ ID NO: 9
  Mouse siRNA si2 (5′)

SEQ ID NO: 29 - DNA, 59 bp, Artificial Sequence
  Mouse siRNA si2 (3′)
ttccaaaaaactccaggtggaatggccctctcttgaagggccattccacctggagtggg

SEQ ID NO: 30 - DNA, 64 bp, Artificial Sequence
  Human si RNA H1 (5′)
gatccccccctgatgtattggacattttcaagagaaatgtccaatacatcagggtttttggaaa

SEQ ID NO: 31 - DNA, 64 bp, Artificial Sequence
  Human si RNA H1 (3′)
agcttttccaaaaaccctgatgtattggacatttctcttgaaaatgtccaatacatcagggggg

SEQ ID NO: 32 - DNA, 64 bp, Artificial Sequence
  Human siRNA H2 (5′)
gatccccacttctaagaaagtccaccttcaagagaggtggtctttcttagaagttttttggaaa

SEQ ID NO: 33 - DNA, 64 bp, Artificial Sequence
  Human siRNA H2 (3′)
agcttttccaaaaaacttctaagaaagtccacctctcttgaaggtggtctttcttagaagtggg

SEQ ID NO: 34 - DNA, 56 bp, Artificial Sequence
  HpaRz1 template
agcacctttcggcctttcggcctcatcagctcaagacctatagtgagtcgtattac

SEQ ID NO: 35 - DNA, 55 bp, Artificial Sequence
  HpaRz2 template
acctactttcggcctttcggcctcatcagaagaagcctatagtgagtcgtattac

SEQ ID NO: 36 - DNA, 56 bp, Artificial Sequence
  Hpa Rz3 template
ctgctctttcggcctttcggcctcatcagccaagggcctatagtgagtcgtattac

SEQ ID NO: 37 - DNA, 55 bp, Artificial Sequence
  Hpa Rz4 template
caaaactttcggcctttcggcctcatcagtatggtcctatagtgagtcgtattac

SEQ ID NO: 38 - DNA, 56 bp, Artificial Sequence
  Hpa Rz5 template
aaactctttcggcctttcggcctcatcagtggtcctcctatagtgagtcgtattac

SEQ ID NO: 39 - DNA, 56 bp, Artificial Sequence
  Hpa Rz6 template
gctggatttcggcctttcggcctcatcagaattgggcctatagtgagtcgtattac

SEQ ID NO: 40 - DNA, 1477 bp, Homo sapiens
tgacgccaacctggccacggacccgcggttcctcatcctcctgggttctccaaagcttcgtaccttggccagaggcttgtctcctgcgtacctgaggtttggtggcaccaagacagacttcctaattttcgatcccaagaaggaatcaacctttgaagagagaagttactggcaatctcaagtcaaccaggatatttgcaaatatggatccatccctcctgatgtggaggagaagttacggttggaatggccctaccaggagcaattgctactccgagaacactaccagaaaaagttcaagaacagcacctactcaagaagctctgtagatgtgctatacacttttgcaaactgctcaggactggacttgatctttggcctaaatgcgttattaagaacagcagatttgcagtggaacagttctaatgctcagttgctcctggactactgctcttccaaggggtataacatttcttgggaactaggcaatgaacctaacagtttccttaagaaggctgatattttcatcaatgggtcgcagttaggagaagattttattcaattgcataaacttctaagaaagtccaccttcaaaaatgcaaaactctatggtcctgatgttggtcagcctcgaagaaagacggctaagatgctgaagagcttcctgaaggctggtggagaagtgattgattcagttacatggcatcactactatttgaatggacggactgctaccagggaagattttctaaaccctgatgtattggacatttttatttcatctgtgcaaaaagttttccaggtggttgagagcaccaggcctggcaagaaggtctggttaggagaaacaagctctgcatatggaggcggagcgcccttgctatccgacacctttgcagctggctttatgtggctggataaattgggcctgtcagcccgaatgggaatagaagtggtgatgaggcaagtattctttggagcaggaaactaccatttagtggatgaaaacttcgatcctttacctgattattggctatctcttctgttcaagaaattggtgggcaccaaggtgttaatggcaagcgtgcaaggttcaaagagaaggaagcttcgagtataccttcattgcacaaacactgacaatccaaggtataaagaaggagatttaactctgtatgccataaacctccataacgtcaccaagtacttgcggttaccctatcctttttctaacaagcaagtggataaataccttctaagacctttgggacctcatggattactttccaaatctgtccaactcaatggtctaactctaaagatggtggatgatcaaaccttgccacctttaatggaaaaacctctccggccaggaagttcactgggcttgccagctttctcatatagtttttttgtgataagaaatgccaaagttgctgcttgcatctgaaaataaaatatactagtcctgacactg

SEQ ID NO: 41 - DNA, 54 bp, Artificial Sequence
  5′ Primer for generating the anti-hpa siRNA pSi2-lenti
tactccaggtggaatggcccttcaagagagggccattccacctggagttttttc

SEQ ID NO: 42 - DNA, 57 bp, Artificial Sequence
  3′ Primer for generating the anti-hpa siRNA pSi2-lenti
tcgagaaaaactccaggtggaatggccctctcttgaagggccattccacctggagta

SEQ ID NO: 43 - protein, 14 aa, Artificial Sequence
  Peptide for raising anti-heparanase antibody
KKFKNSTYRSSSVD

SEQ ID NO: 44 - DNA, 15 bp, Homo sapiens
agcacctactcaaga

SEQ ID NO: 45 - DNA, 15 bp, Homo sapiens
acctactcaagaagc

SEQ ID NO: 46 - DNA, 15 bp, Homo sapiens
ctgctcttccaaggg

SEQ ID NO: 47 - DNA, 15 bp, Homo sapiens
caaaactctatggtc

SEQ ID NO: 48 - DNA, 15 bp, Homo sapiens
aaactctatggtcct

SEQ ID NO: 49 - DNA, 15 bp, Homo sapiens
gctggataaattggg

SEQ ID NO: 50 - DNA, 44 bp, Artificial Sequence
  Oligonucleotide for pcDNA3 insert - Rz4 - antisense
ggccgccaaaactttcggcctttcggcctcatcagtatggtcca

SEQ ID NO: 51 - DNA, 44 bp, Artificial Sequence
  Oligonucleotide for pcDNA insert - Rz5 - antisense
ggccgcaaactctttcggcctttcggcctcatcagtggtcctca

SEQ ID NO: 52 - DNA, 44 bp, Artificial
  Oligonucleotide for pcDNA3 insert - Rz4 - sense
agcttggaccatactgatgaggccgaaaggccgaaagttttggc

SEQ ID NO: 53 - DNA, 44 bp, Artificial sequence
  Oligonucleotide for pcDNA3 insert - Rz5 - sense
agcttgaggaccactgatgaggccgaaaggccgaaagagtttgc

SEQ ID NO: 54 - DNA, 1746 bp, Mus musculus
gagaggcgaagcaggaccggttgcagggggcttgagccagcgcgccgggctgccccagctctcccggcagcgggcggtccagccaggtgggatgctgaggctgctgctgctgtggctctgggggccgctcggtgccctggcccagggcgcccccgcggggaccgcgccgaccgacgacgtggtagacttggagttttacaccaagcggccgctccgaagcgtgagtccctcgttcctgtccatcaccatcgacgccagcctggccaccgacccgcgcttcctcaccttcctgggctctccaaggctccgtgctctggctagaggcttatctcctgcatacttgagatttggcggcacaaagactgacttccttatttttgatccggacaaggaaccgacttccgaagaaagaagttactggaaatctcaagtcaaccatgatatttgcaggtctgagccggtctctgctgcggtgttgaggaaactccaggtggaatggcccttccaggagctgttgctgctccgagagcagtaccaaaaggagttcaagaacagcacctactcaagaagctcagtggacatgctctacagttttgccaagtgctcggggttagacctgatctttggtctaaatgcgttactacgaaccccagacttacggtggaacagctccaacgcccagcttctccttgactactgctcttccaagggttataacatctcctgggaactgggcaatgagcccaacagtttctggaagaaagctcacattctcatcgatgggttgcagttaggagaagactttgtggagttgcataaacttctacaaaggtcagctttccaaaatgcaaaactctatggtcctgacatcggtcagcctcgagggaagacagttaaactgctgaggagtttcctgaaggctggcggagaagtgatcgactctcttacatggcatcactattacttgaatggacgcatcgctaccaaagaagattttctgagctctgatgtgctggacacttttattctctctgtgcaaaaaattctgaaggtcactaaagagatcacacctggcaagaaggtctggttgggagagacgagctcagcttacggtggcggtgcacccttgctgtccaacacctttgcagctggctttatgtggctggataaattgggcctgtcagcccagatgggcatagaagtcgtgatgaggcaggtgttcttcggagcaggcaactaccacttagtggatgaaaactttgagcctttacctgattactggctctctcttctgttcaagaaactggtaggtcccagggtgttactgtcaagagtgaaaggcccagacaggagcaaactccgagtgtatctccactgcactaacgtctatcacccacgatatcaggaaggagatctaactctgtatgtcctgaacctccataatgtcaccaagcacttgaaggtaccgcctccgttgttcaggaaaccagtggatacgtaccttctgaagccttcggggccggatggattactttccaaatctgtccaactgaacggtcaaattctgaagatggtggatgagcagaccctgccagctttgacagaaaaacctctccccgcaggaagtgcactaagcctgcctgccttttcctatggtttttttgtcataagaaatgccaaaattgctgcttgtatatgaaaataaaaggcatacggtacccctgagacaaaaaaaaaaaaaaaaaa

SEQ ID NO: 55 - DNA, 55 bp, Artificial Sequence
  Sense strand of anti-heparanase ribozyme (HpaRz2)
gtaatacgactcactataggcttcttctgatgaggccgaaaggccgaaagtaggt

SEQ ID NO: 56 - RNA, 15 nt, Homo sapiens
accuacucaagaagc

SEQ ID NO: 57 - DNA, 1758 bp, Homo sapiens
aggagaaaagggcgctggggctcggcgggaggaagtgctagagctctcgactctccgctgcgcggcagctggcggggggagcagccaggtgagcccaagatgctgctgcgctcgaagcctgcgctgccgccgccgctgatgctgctgctcctggggccgctgggtcccctctcccctggcgccctgccccgacctgcgcaagcacaggacgtcgtggacctggacttcttcacccaggagccgctgcacctggtgagcccctcgttcctgtccgtcaccattgacgccaacctggccacggacccgcggttcctcatcctcctgggttctccaaagcttcgtaccttggccagaggcttgtctcctgcgtacctgaggtttggtggcaccaagacagacttcctaattttcgatcccaagaaggaatcaacctttgaagagagaagttactggcaatctcaagtcaaccaggatatttgcaaatatggatccatccctcctgatgtggaggagaagttacggttggaatggccctaccaggagcaattgctactccgagaacactaccagaaaaagttcaagaacagcacctactcaagaagctctgtagatgtgctatacacttttgcaaactgctcaggactggacttgatctttggcctaaatgcgttattaagaacagcagatttgcagtggaacagttctaatgctcagttgctcctggactactgctcttccaaggggtataacatttcttgggaactaggcaatgaacctaacagtttccttaagaaggctgatattttcatcaatgggtcgcagttaggagaagattttattcaattgcataaacttctaagaaagtccaccttcaaaaatgcaaaactctatggtcctgatgttggtcagcctcgaagaaagacggctaagatgctgaagagcttcctgaaggctggtggagaagtgattgattcagttacatggcatcactactatttgaatggacggactgctaccagggaagattttctaaaccctgatgtattggacatttttatttcatctgtgcaaaaagttttccaggtggttgagagcaccaggcctggcaagaaggtctggttaggagaaacaagctctgcatatggaggcggagcgcccttgctatccgacacctttgcagctggctttatgtggctggataaattgggcctgtcagcccgaatgggaatagaagtggtgatgaggcaagtattctttggagcaggaaactaccatttagtggatgaaaacttcgatcctttacctgattattggctatctcttctgttcaagaaattggtgggcaccaaggtgttaatggcaagcgtgcaaggttcaaagagaaggaagcttcgagtataccttcattgcacaaacactgacaatccaaggtataaagaaggagatttaactctgtatgccataaacctccataacgtcaccaagtacttgcggttaccctatcctttttctaacaagcaagtggataaataccttctaagacctttgggacctcatggattactttccaaatctgtccaactcaatggtctaactctaaagatggtggatgatcaaaccttgccacctttaatggaaaaacctctccggccaggaagttcactgggcttgccagctttctcatatagtttttttgtgataagaaatgccaaagttgctgcttgcatctgaaaataaaatatactagtcctgacactg